(19) United States
(12) Patent Application Publication
(10) Pub. No.: US 2023/0416394 A1
(43) Pub. Date: Dec. 28, 2023

(54) NOVEL CONJUGATE MOLECULES TARGETING CD39 AND TGFBETA

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Dawei SUN, Shanghai (CN); Zhihao WU, Shanghai (CN); Jun SUN, Shanghai (CN); Yanan GENG, Shanghai (CN); Rui GAO, Shanghai (CN); Lili TANG, Shanghai (CN); Qinglin DU, Shanghai (CN); Yangsheng QIU, Shanghai (CN); Robert H. ARCH, Shanghai (CN); Hongtao LU, Shanghai (CN)

(21) Appl. No.: 18/254,606

(22) PCT Filed: Nov. 25, 2021
(86) PCT No.: PCT/CN2021/133083

§ 371 Date: May 26, 2023

(30) Foreign Application Priority Data
Nov. 27, 2020 (WO) PCT/CN2020/132392
Nov. 23, 2021 (CN) 202111396829.4

(51) Int. Cl. C07K 16/28; C07K 16/22; C12N 15/63; A61P 35/00

(52) U.S. Cl. CPC C07K 16/2896; C07K 16/22; C12N 15/63; A61P 35/00; C07K 2317/565; C07K 2317/567; C07K 2317/92; C07K 2317/31

(57) ABSTRACT

Provided are conjugate molecules comprising a CD39 inhibitory portion capable of interfering interaction between CD39 and its substrate, and a TGF β inhibitory portion capable of interfering interaction between TGF β and its receptor, isolated polynucleotides encoding the same, pharmaceutical compositions comprising the same and the uses thereof.

### FIELD OF THE INVENTION

The present disclosure generally relates to novel conjugate molecules targeting CD39 and TGFβ.

### BACKGROUND

CD39, also known as ecto-nucleoside triphosphate diphosphohydrolase-1 (ENTPDase1), is an integral membrane protein that converts ATP or ADP into AMP, and then CD73 dephosphorylates AMP into adenosine, which is a potent immunosuppressor and binds to adenosine receptors (for example, A2A receptor) at the surface of CD4⁺, CD8⁺ T cells and natural killer (NK) cells, and inhibits T-cell and NK-cell responses, thereby suppressing the immune system. Adenosine also binds to A2A or A2B receptors on macrophages and dendritic cells, inhibits phagocytosis and antigen presentation and increases secretion of pro-tumorigenic factors, such as VEGF, TGFβ, and IL-6. The enzymatic activities of CD39 and CD73 play strategic roles in calibrating the duration, magnitude, and chemical nature of purinergic signals delivered to immune cells through the conversion of ADP and ATP to AMP and AMP to adenosine, respectively (Luca Antonioli et al., Trends Mol Med. 2013 June; 19(6):355-367). Increased adenosine levels mediated by CD39 and CD73 generate an immunosuppressive environment which promotes the development and progression of cancer.

Transforming growth factor beta (TGFβ) is a pleiotropic cytokine that is expressed at elevated levels in late-stage primary and metastatic tumors, and activates both anti-proliferative and tumor-promoting signaling cascades. Tumor stromal cells and many types of tumors, including breast, colon, lung, pancreas, prostate, as well as hematologic malignancies, produced high levels of TGFβ. Besides of promoting epithelial-to-mesenchymal transition (EMT), invasion, and metastases of tumor cells, TGFβ enables tumors to evade immune surveillance through the mechanisms such as suppressing the expression of interferon-γ (IFN-γ), restricting the differentiation of Th1 cells and attenuating the function of CD8⁺ effector cells. Most significantly, TGFβ induces the differentiation of regulatory T cells (Tregs). Tregs further inhibit inflammation through the production of immunosuppressive cytokines (IL-10, TGFβ, and IL-35), the expression of inhibitory molecules (CTLA-4) and by hydrolyzing ATP to adenosine through the CD39.

Given the roles of CD39 and TGFβ in modulating immune responses to tumors, needs remain for therapeutic agents that antagonize CD39 activity, or both CD39 and TGFβ activities for the treatment of diseases, e.g. cancers.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides a conjugate molecule comprising a CD39 inhibitory portion capable of interfering interaction between CD39 and its substrate, and a TGFβ inhibitory portion capable of interfering interaction between TGFβ and its receptor.

In certain embodiments, the CD39 inhibitory portion is capable of interfering interaction between CD39 and ATP/ADP, and/or the TGFβ inhibitory portion is capable of interfering interaction between TGFβ and TGFβ receptor. In certain embodiments, the CD39 inhibitory portion is an antagonist of CD39 selected from a group consisting of a CD39-binding agent, an RNAi that targets an encoding sequence of CD39, an antisense nucleotide that targets an encoding sequence of CD39, and an agent that competes with CD39 to bind to its substrate. In certain embodiments, the TGFβ inhibitory portion is an antagonist of TGFβ selected from a group consisting of a TGFβ-binding agent, an RNAi that targets an encoding sequence of TGFβ, an antisense nucleotide that targets an encoding sequence of TGFβ, and an agent that competes with TGFβ to bind to its receptor. In certain embodiments, the CD39-binding agent is selected from the group consisting of an antibody or an antigen-binding fragment thereof that specifically recognizes CD39, and a small molecule compound that binds to CD39, and/or the TGFβ-binding agent is selected from the group consisting of an antibody or an antigen-binding fragment thereof that specifically recognizes TGFβ, and a small molecule compound that binds to TGFβ.

In certain embodiments, the conjugate molecule is a fusion protein comprising a CD39-binding domain linked to a TGFβ-binding domain. In certain embodiments, the TGFβ-binding domain binds to human and/or mouse TGFβ. In certain embodiments, the TGFβ-binding domain binds to human TGFβ1, human TGFβ2, and/or human TGFβ3. In certain embodiments, the TGFβ-binding domain comprises an extracellular domain (ECD) of a TGFβ receptor. In certain embodiments, the TGFβ receptor is TGFβ Receptor I (TGFβRI), TGFβ Receptor II (TGFβRII), or TGFβ Receptor III (TGFβRIII). In certain embodiments, the ECD comprises an amino acid sequence of SEQ ID NO: 163, SEQ ID NO: 164, SEQ ID NO: 165, or an amino acid sequence having at least 85% sequence identity thereof yet retaining binding specificity to TGFβ. In certain embodiments, the TGFβ-binding domain comprises two or more ECDs of a TGFβ receptor. In certain embodiments, the two or more ECDs are derived from the same TGFβ receptor, or are derived from at least two different TGFβ receptors. In certain embodiments, the two or more ECDs comprise a first ECD derived from TGFβRI and a second ECD derived from TGFβRII. In certain embodiments, the two or more ECDs are operably linked in series. In certain embodiments, the two or more ECDs are linked via a first linker. In certain embodiments, the TGFβ-binding domain comprises an amino acid sequence selected from the group consisting of SEQ ID NO: 166, SEQ ID NO: 167, SEQ ID NO: 168, SEQ ID NO: 169, SEQ ID NO: 170, SEQ ID NO: 171, or any combination thereof.

In certain embodiments, the CD39-binding domain binds to human CD39. In certain embodiments, the TGFβ-binding domain is linked to the CD39 binding domain via a second linker. In certain embodiments, the CD39-binding domain comprises an anti-CD39 antibody moiety. In certain embodiments, the anti-CD39 antibody moiety comprises a heavy chain variable region and a light chain variable region. In certain embodiments, the anti-CD39 antibody moiety further comprises a heavy chain constant domain appended to a carboxyl terminus of the heavy chain variable region. In certain embodiments, the anti-CD39 antibody moiety further comprises a light chain constant domain appended to a carboxyl terminus of the light chain variable region. In certain embodiments, the TGFβ-binding domain is linked to the anti-CD39 antibody moiety at a position selected from the group consisting of: 1) amino terminus of the heavy chain variable region, 2) amino terminus of the light chain variable region, 3) carboxyl terminus of the heavy chain variable region; 4) carboxyl terminus of the light chain variable region; 5) carboxyl terminus of the heavy chain constant region; and 6) carboxyl terminus of the light chain constant region, of the anti-CD39 antibody moiety.

In certain embodiments, the fusion protein comprises two or more TGFβ-binding domains which are (i) all linked to the heavy chain variable region of the anti-CD39 antibody moiety, or (ii) are all linked to the light chain variable region of the anti-CD39 antibody moiety. In certain embodiments, the fusion protein comprises two or more TGFβ-binding domains which are linked to the heavy and the light chain variable region of anti-CD39 antibody moiety, respectively. In certain embodiments, the fusion protein comprises two or more TGFβ-binding domains which are all linked to the heavy chain constant region of the anti-CD39 antibody moiety. In certain embodiments, the fusion protein comprises two or more TGFβ-binding domains which are all linked to the light chain constant region of anti-CD39 antibody moiety. The fusion protein comprises two or more TGFβ-binding domains which are linked to the heavy and the light chain constant regions of the anti-CD39 antibody moiety, respectively.

In certain embodiments, the fusion protein comprises two, three, four, five, six or more TGFβ-binding domains. In certain embodiments, the first and/or the second linker is selected from the group consisting of a cleavable linker, a non-cleavable linker, a peptide linker, a flexible linker, a rigid linker, a helical linker, and a non-helical linker. In certain embodiments, the first and/or the second linker comprises a peptide linker. In certain embodiments, the peptide linker comprises a GS linker. In certain embodiments, the GS linker comprises one or more repeats of SEQ ID NO: 177 (GGGS) or SEQ ID NO: 173 (GGGGS). In certain embodiments, the peptide linker comprises an amino acid sequence of GGGGSGGGGSGGGGSG (SEQ ID NO: 182).

In another aspect, the present disclosure provides a pharmaceutical composition comprising the conjugate molecule of the present disclosure, and one or more pharmaceutically acceptable carriers. In another aspect, the present disclosure provides an isolated polynucleotide encoding the conjugate molecule of the present disclosure. In another aspect, the present disclosure provides a vector comprising the isolated polynucleotide of the present disclosure. In another aspect, the present disclosure provides a host cell comprising the vector of the present disclosure. In another aspect, the present disclosure provides a kit comprising the conjugate molecule of the present disclosure and/or the pharmaceutical composition of the present disclosure, and a second therapeutic agent.

In another aspect, the present disclosure provides a method of expressing the conjugate molecule of the present disclosure, comprising culturing the host cell of the present disclosure under the condition at which the vector of the present disclosure is expressed. In another aspect, the present disclosure provides a method of treating, preventing or alleviating a CD39 related and/or a TGFβ related disease, disorder or condition in a subject, comprising administering to the subject a therapeutically effective amount of the conjugate molecule of the present disclosure and/or the pharmaceutical composition of the present disclosure. In another aspect, the present disclosure provides a method of treating, preventing or alleviating a disease treatable by reducing the ATPase activity of CD39 in a subject, comprising administering to the subject a therapeutically effective amount of the conjugate molecule of the present disclosure and/or the pharmaceutical composition of the present disclosure. In another aspect, the present disclosure provides a method of treating, preventing or alleviating a disease associated with adenosine-mediated inhibition of T, Monocyte, Macrophage, DC, APC, NK and/or B cell activity in a subject, comprising administering to the subject a therapeutically effective amount of the conjugate molecule of the present disclosure and/or the pharmaceutical composition of the present disclosure. In another aspect, the present disclosure provides a method of modulating CD39 activity in a CD39-positive cell, comprising exposing the CD39-positive cell to the conjugate molecule of the present disclosure and/or the pharmaceutical composition of the present disclosure.

In another aspect, the present disclosure provides a method of treating, preventing or alleviating a disease associated with an increased level and/or activity of TGFβ in a subject, comprising administering to the subject a therapeutically effective amount of the conjugate molecule of the present disclosure and/or the pharmaceutical composition of the present disclosure. In another aspect, the present disclosure provides use of the conjugate molecule of the present disclosure and/or the pharmaceutical composition of the present disclosure in the manufacture of a medicament for treating, preventing or alleviating a CD39 related or a TGFβ related disease, disorder or condition in a subject.

### BRIEF DESCRIPTION OF THE DRAWINGS

FIG. 1 shows blockade of ATP-mediated suppression of T cell proliferation by anti-CD39 monoclonal antibodies mAb21 and mAb23. mIgG2a was used as an isotype control antibody.

FIG. 2 shows blockade of ATP-mediated suppression of T cell proliferation by anti-CD39 chimeric antibodies c14, c19, c21 and c23. hIgG4 refers to the human IgG4 isotype control antibody.

FIG. 3 shows the CD39 expression level on dendritic cells (DC).

FIGS. 4A to 4C show ATP-mediated DC activation by anti-CD39 chimeric antibodies c14, c19, c21 and c23, as measured by CD86 (FIG. 4A), CD83 (FIG. 4B) and HLA-DR (FIG. 4C) expression using FACS.

FIG. 5 shows tumor growth after treatment with anti-CD39 chimeric antibodies c23-hIgG4 and c23-hIgG1 in mice inoculated with MOLP-8 cells (human multiple myeloma cell line).

FIG. 6A shows the binding property of humanized antibody hu23.H5L5 to ENTPD1 (i.e. CD39), ENTPD2 (i.e. CD39L1), ENTPD3 (i.e. CD39L3), ENTPD5 (i.e. CD39L4) and ENTPD6 (i.e. CD39L2) proteins, respectively. FIG. 6B shows the binding of negative control hIgG4 with ENTPD1 (i.e. CD39), ENTPD2 (i.e. CD39L1), ENTPD3 (i.e. CD39L3), ENTPD5 (i.e. CD39L4) and ENTPD6 (i.e. CD39L2) proteins, respectively.

FIGS. 7A and 7B show binding activity of c23 humanized antibodies with MOLP-8 cells by FACS.

FIG. 8 shows binding activity of c23 humanized antibodies (obtained by yeast display) with MOLP-8 cells.

FIGS. 9A and 9B show ATPase inhibition of c23 humanized antibodies on SK-MEL-28 cells by FACS.

FIGS. 10A to 10C show binding activity of c14 humanized antibodies with MOLP-8 cells by FACS.

FIGS. 11A to 11D show ATP-mediated T cell activation in PBMC by humanized antibody hu23.H5L5, as measured by IL-2 (FIG. 11A), IFN-γ (FIG. 11B), CD4⁺ T cell proliferation (FIG. 11C) and CD8⁺ T cell proliferation (FIG. 11D).

FIGS. 12A to 12E show binding activity of humanized antibodies hu23.H5L5 and hu14.H1L1 with SK-MEL-5 (FIG. 12A), SK-MEL-28 (FIG. 12B), MOLP-8 (FIG. 12C), CHOK1-cynoCD39 (FIG. 12D) and CHOK1-mCD39 (FIG. 12E) cells by FACS.

FIGS. 13A to 13B show ATPase inhibition activity by humanized antibodies hu23.H5L5 and hu14.H1L1 on SK-MEL-5 cells (FIG. 13A) and MOLP-8 cells (FIG. 13B).

FIGS. 14A to 14C show ATP-mediated monocyte activation by anti-CD39 humanized antibody hu23.H5L5, as measured by CD80 (FIG. 14A), CD86 (FIG. 14B) and CD40 (FIG. 14C) expression.

FIG. 15 shows that humanized antibody hu23.H5L5 increased ATP-mediated DC activation as measured by CD83 expression (FIG. 15A), and enhanced T cell proliferation (FIG. 15B) and T cell activation (FIG. 15C).

FIG. 16 shows the tumor growth inhibition by humanized antibodies hu23.H5L5 and hu14.H1L1 in MOLP-8 xenograft mice.

FIG. 17 shows the tumor growth inhibition of anti-CD39 humanized antibody hu23.H5L5 in NK depleted MOLP-8 xenograft mice.

FIG. 18 shows the tumor growth inhibition of anti-CD39 humanized antibody hu23.H5L5 in macrophage depleted MOLP-8 xenograft mice.

FIG. 19A shows epitope binning results of humanized antibodies hu23.H5L5 and hu14.H1L1 with references antibodies. FIG. 19B shows the epitope grouping of the tested antibodies.

FIG. 20 shows the effect of anti-CD39 humanized antibody hu23.H5L5 on human macrophage IL1β release induced by LPS stimulation.

FIG. 21 shows the tumor growth inhibition of humanized antibody hu23.H5L5 at different dosages (0.03 mg/kg, 0.3 mg/kg, 3 mg/kg, 10 mg/kg, 30 mg/kg) in PBMC adoption mice.

FIG. 22 shows the epitope mapping results of humanized antibody hu23.H5L5, chimeric antibodies c34 and c35, as well as reference antibodies T895, 1394 and 9-8B.

FIGS. 23A to 23C show extracellular ATP inhibited CD8⁺ T cell proliferation reversed by humanized antibody hu23.H5L5, as measured by T cell proliferation (FIG. 23A), CD25⁺ Cells (FIG. 23B), and living cells population (FIG. 23C).

FIGS. 24A to 24G show schematic drawings of the exemplary anti-CD39/TGFβ Trap molecules of the present disclosure.

FIGS. 25A to 25D show the binding property of exemplary anti-CD39/TGFβ Trap molecules to human TGFβ1 (FIG. 25A), human TGFβ2 (FIG. 25B), human TGFβ3 (FIG. 25C) and mouse TGFβ1 (FIG. 25D), respectively.

FIG. 26 shows the blocking assay results of exemplary anti-CD39/TGFβ Trap molecules to human TGFβ1 and TGFβRII.

FIGS. 27A and 27B show the binding activity of exemplary anti-CD39/TGFβ Trap molecules to human CD39 with MOLP-8 cells (FIG. 27A) and CHOK1 cells (FIG. 27B) by FACS, respectively.

FIGS. 28A and 28B show the simultaneous binding activity of exemplary anti-CD39/TGFβ Trap molecules to human CD39 and TGFβ1 by ELISA (FIG. 28A) and FACS (FIG. 28B), respectively.

FIG. 29A shows the TGFβ reporter assay result of exemplary anti-CD39/TGFβ Trap molecules in transfected HEK293 cells. FIG. 29B shows the TGFβ neutralizing activities of exemplary anti-CD39/TGFβ Trap molecules when pre-incubated at different CD39 protein:anti-CD39/TGFβ Trap molecule ratios.

FIGS. 30A to 30C show the ATPase inhibition activity by exemplary anti-CD39/TGFβ Trap molecules on MOLP-8 cells (FIGS. 30A and 30B) and CHO cells (FIG. 30C), respectively.

FIG. 31A shows that the addition of Tregs to autologous T cells primed by allogenic DCs suppressed IFN-γ secretion of T cells. FIGS. 31B to 31D show the effects of exemplary anti-CD39/TGFβ Trap molecules on Treg-mediated suppression of human T cells as measured by CD4⁺ T cell proliferation % (FIG. 31B), CD8⁺ T cell proliferation % (FIG. 31C) and alteration in IFN-γ secretion (FIG. 31D).

FIG. 32 provides a graph depicting the percent inhibition of apoptosis on human T cells treated with the same molar of anti-CD39/TGFβ Trap molecules ES014-1 and ES014-2, anti-CD39 antibody ES014_v2, TGF-beta trap ES014_v1 and control antibody ES014_v3, as indicated. FIG. 32A showed the percentage of early apoptosis on total T cells, wherein the x-axis indicates the antibody and concentrations, and the y-axis shows the percentage (%) of early T cell apoptosis (Annexin V⁺PI⁻). FIG. 32B showed the percentage of late apoptosis on total T cells, wherein the x-axis indicates the antibody and concentrations, and the y-axis shows the percentage (%) of late T cell apoptosis (Annexin V⁺PI⁻).

FIG. 33 provides a graph depicting T cell functions treated with the same molar of anti-CD39/TGFβ Trap molecules ES014-1 and ES014-2, anti-CD39 antibody ES014_v2, TGF-beta trap ES014_v1 and control antibody ES014_v3, as indicated. FIG. 33A showed the FACS plot for cell viability and cell activation, and FIG. 33B showed IL-2 and IFN-γ production in supernatant.

FIG. 34 provides a graph depicting the Foxp3 expression on T cells treated with anti-CD39/TGFβ Trap molecules ES014-1 and ES014-2, anti-CD39 antibody ES014_v2, TGF-beta trap ES014_v1 and control antibody ES014_v3, as indicated. FIG. 34A showed the percentage of Foxp3 expression on CD4⁺ T cells, and FIG. 34B showed the percentage of Foxp3 expression on CD8⁺ T cells.

FIG. 35 provides a graph depicting the percentage (%) divided of total T cells treated with anti-CD39/TGFβ Trap molecules ES014-1 and ES014-2, anti-CD39 antibody ES014_v2, TGF-beta trap ES014_v1 and control antibody ES014_v3, as indicated. FIG. 35A showed the percentage (%) divided of CD4+ T cells, and FIG. 35B showed the percentage (%) divided of CD8⁺ T cells.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or η, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragments include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab” refers to a Fab fragment that includes a portion of the hinge region. “F(ab′)₂” refers to a dimer of Fab′.

“Fc” with regard to an antibody (e.g. of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V H domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1): 93-101 (2003)). The variable domain of a heavy chain antibody (VHH domain) represents the smallest known antigen-binding unit generated by adaptive immune responses (Koch-Nolte F. et al., FASEB J. November; 21(13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent. In some embodiments, the antibody or an antigen-binding fragment thereof is tetravalent.

As used herein, a “bispecific” antibody refers to an artificial antibody which has fragments derived from two different monoclonal antibodies or derived from one antibody and another protein (e.g. TGFβ receptor), and is capable of binding to two different epitopes. The two epitopes may present on the same antigen, or they may present on two different antigens.

In certain embodiments, an “scFv dimer” is a bivalent diabody or bispecific scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two VH moieties linked by a peptide linker (e.g. a long flexible linker) and bound to two VL moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K D may be determined by using any conventional method known in the art, including but are not limited to surface plasmon resonance method, Octet method, microscale thermophoresis method, HPLC-MS method and FACS assay method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹ M, ≤3×10⁻⁹ M, ≤2×10⁻⁹ M, or ≤10⁻⁹ M) can indicate specific binding between an antibody or antigen binding fragments thereof and CD39 (e.g. human CD39).

The ability to “compete for binding to human CD39” as used herein refers to the ability of a first antibody or antigen-binding fragment to inhibit the binding interaction between human CD39 and a second anti-CD39 antibody to any detectable degree. In certain embodiments, an antibody or antigen-binding fragment that compete for binding to human CD39 inhibits the binding interaction between human CD39 and a second anti-CD39 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e. including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The terms “polypeptide”, “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter.

Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a CD39 related disease, or refer to identification of a subject with a CD39 related disease who may benefit from a particular treatment regimen. In some embodiments, diagnosis contains the identification of abnormal amount or activity of CD39. In some embodiments, diagnosis refers to the identification of a cancer or an autoimmune disease in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

“CD39” as used herein, also known as ENTPD1 or ENTPDase1, refers to an integral membrane protein that coverts ATP to AMP. Structurally, it is characterized by two transmembrane domains, a small cytoplasmic domain, and a large extracellular hydrophobic domain. In certain embodiments, the CD39 is human CD39. CD39 as used herein may be from other animal species, such as from mouse, and cynomolgus, among others. Exemplary sequence of human CD39 protein is disclosed in NCBI Ref Seq No. NP_001767.3. Exemplary sequence of Mus musculus (mouse) CD39 protein is disclosed in NCBI Ref Seq No. NP_033978.1. Exemplary sequence of Cynomolgus (monkey) CD39 protein is disclosed in NCBI Ref Seq No. XP_015311945.1.

In addition to CD39, the ENTPDase family also comprise several other members, including, ENTPDases 2, 3, 4, 5, 6, 7, and 8 (also known as ENTPD2, 3, 4, 5, 6, 7, and 8, and are used interchangeably in the present disclosure). Four of the ENTPDases are typical cell surface-located enzymes with an extracellularly facing catalytic site (ENTPDase1, 2, 3, 8). ENTPDases 5 and 6 exhibit intracellular localization and undergo secretion after heterologous expression. ENTPDases 4 and 7 are entirely intracellularly located, facing the lumen of cytoplasmic organelles. In some embodiments, the antibody or an antigen-binding fragment thereof provided herein specifically bind to CD39 (i.e. ENTPDase 1), but does not bind to the other family members, for example, ENTPDases 2, 3, 5, or 6.

The term “anti-CD39 antibody moiety” refers to an antibody (including an antigen-binding fragment thereof) that is capable of specific binding to CD39 (e.g. human or monkey CD39), and forms a portion of the conjugate molecule targeting both CD39 and TGFβ. The term “anti-human CD39 antibody moiety” refers to an antibody (including an antigen-binding fragment thereof) that is capable of specific binding to human CD39, and forms a portion of the conjugate molecule targeting both human CD39 and TGFβ.

A “CD39 related” disease, disorder or condition as used herein refers to any disease or condition caused by, exacerbated by, or otherwise linked to increased or decreased expression or activities of CD39. In some embodiments, the CD39 related disease, disorder or condition is an immune-related disorder, such as, for example, an autoimmune disease. In some embodiments, the CD39 related disease, disorder or condition is a disorder related to excessive cell proliferation, such as, for example, cancer. In certain embodiments, the CD39 related disease or condition is characterized in expressing or over-expressing of CD39 and/or CD39 related genes such as ENTPD1, 2, 3, 4, 5, 6, 7, or 8 genes.

The terms “transforming growth factor beta” and “TGFβ” as used herein refer to any of the TGFβ family proteins that have either the full-length, native amino acid sequence of any of the TGF-betas from subjects (e.g. human), including the latent forms and associated or unassociated complex of precursor and mature TGFβ (“latent TGFβ”). Reference to such TGFβ herein will be understood to be a reference to any one of the currently identified forms, including TGFβ1, TGFβ2, TGFβ3 isoforms and latent versions thereof, as well as to human TGFβ species identified in the future, including polypeptides derived from the sequence of any known TGFβ and being at least about 75%, preferably at least about 80%, more preferably at least about 85%, still more preferably at least about 90%, and even more preferably at least about 95% homologous with the sequence. The specific terms “TGFβ1,” “TGFβ2,” and “TGFβ3” refer to the TGF-betas defined in the literature, e.g., Derynck et al., Nature, Cancer Res., 47: 707 (1987); Seyedin et al., J. Biol. Chem., 261: 5693-5695 (1986); deMartin et al., EMBO J., 6: 3673 (1987); Kuppner et al., Int. J. Cancer, 42: 562 (1988). The terms “transforming growth factor beta”, “TGFβ”, “TGFbeta”, “TGF-β”, “TGF-beta”, “TGFb”, “TGF-b”, “TGFB”, and “TGF-B” are used interchangeably in the present disclosure.

As used herein, the term “human TGFβ1” refers to a TGFβ1 protein encoded by a human TGFB1 gene (e.g., a wild-type human TGFB1 gene). An exemplary wild-type human TGFβ1 protein is provided by GenBank Accession No. NP_000651.3. As used herein, the term “human TGFβ2” refers to a TGFβ2 protein encoded by a human TGFB2 gene (e.g., a wild-type human TGFB2 gene). Exemplary wild-type human TGFβ2 proteins are provided by GenBank Accession Nos. NP_001129071.1 and NP_003229.1. As used herein, the term “human TGFβ3” refers to a TGFβ3 protein encoded by a human TGFB3 gene (e.g., a wild-type human TGFB3 gene). Exemplary wild-type human TGFβ3 proteins are provided by GenBank Accession Nos. NP_003230.1, NP_001316868.1, and NP_001316867.1.

As used herein, the terms “mouse TGFβ1”, “mouse TGFβ2”, and “mouse TGFβ3” refer to a TGFβ1 protein, TGFβ2 protein, and TGFβ3 protein encoded by a mouse TGFB1 gene (e.g., a wild-type mouse TGFB1 gene), mouse TGFB2 gene (e.g., a wild-type mouse TGFB2 gene), and mouse TGFB3 gene (e.g., a wild-type mouse TGFB3 gene), respectively. Exemplary wild-type mouse (Mus musculus) TGFβ1 protein are provided by GenBank Accession Nos. NP_035707.1 and CAA08900.1. An exemplary wild-type mouse TGFβ2 protein is provided by GenBank Accession No. NP_033393.2. An exemplary wild-type mouse TGFβ3 protein is provided by GenBank Accession No. AAA40422.1.

The term “TGFβ receptor” as used herein refers to any receptor that binds at least one TGFβ isoform. Generally, the TGFβ receptor includes TGFβ Receptor I (TGFβRI), TGFβ Receptor II (TGFβRII), or TGFβ Receptor III (TGFβRIII).

With regard to human, the term “TGFβ Receptor I” or “TGFβRI” refers to a polypeptide having the wild-type human TGFβ Receptor Type 1 sequence (e.g. the amino acid sequence of GenBank Accession No. ABD46753.1), or having a sequence substantially identical to the amino acid sequence of GenBank Accession No. ABD46753.1. The TGFβRI may retain at least 0.1%, at least 0.5%, at least 1%, at least 5%, at least 10%, at least 25%, at least 35%, at least 50%, at least 75%, at least 90%, at least 95%, or at least 99% of the TGFβ-binding activity of the wild-type sequence. The polypeptide of expressed TGFβRI lacks the signal sequence.

With regard to human, the term “TGFβ Receptor II” or “TGFβRII” refers to a polypeptide having the wild-type human TGFβ Receptor Type 2 Isoform A sequence (e.g., the amino acid sequence of GenBank Accession No. NP_001020018.1), or a polypeptide having the wild-type human TGFβ Receptor Type 2 Isoform B sequence (e.g., the amino acid sequence of GenBank Accession No. NP_003233.4), or having a sequence of at least 80%, at least 85%, at least 90%, at least 91%, at least 92%, at least 93%, at least 94%, at least 95%, at least 96%, at least 97%, at least 98%, at least 99% sequence identity to the amino acid sequence of GenBank Accession No. NP 001020018.1 or of GenBank Accession No. NP_003233.4. The TGFβRII may retain at least 0.1%, at least 0.5%, at least 1%, at least 5%, at least 10%, at least 25%, at least 35%, at least 50%, at least 75%, at least 90%, at least 95%, or at least 99% of the TGFβ-binding activity of the wild-type sequence. The polypeptide of expressed TGFβRII lacks the signal sequence.

With regard to human, the term “TGFβ Receptor III” or “TGFβRIII” refers to a polypeptide having the wild-type human TGFβ Receptor Type 3 Isoform A sequence (e.g., the amino acid sequence of GenBank Accession No. NP_003234.2), or a polypeptide having the wild-type human TGFβ Receptor Type 3 Isoform B sequence (e.g., the amino acid sequence of GenBank Accession No. NP_001182612.1), or having a sequence of at least 80%, at least 85%, at least 90%, at least 91%, at least 92%, at least 93%, at least 94%, at least 95%, at least 96%, at least 97%, at least 98%, at least 99% sequence identity to the amino acid sequence of GenBank Accession No. NP_003234.2 and NP_001182612.1. The TGFβRIII may retain at least 0.1%, at least 0.5%, at least 1%, at least 5%, at least 10%, at least 25%, at least 35%, at least 50%, at least 75%, at least 90%, at least 95%, or at least 99% of the TGFβ-binding activity of the wild-type sequence. The polypeptide of expressed TGFβRIII lacks the signal sequence.

A “TGFβ related” disease, disorder or condition as used herein refers to any disease or condition caused by, exacerbated by, or otherwise linked to increased or decreased expression or activities of TGFβ. In some embodiments, the TGFβ related disease, disorder or condition is an immune-related disorder, such as, for example, an autoimmune disease. In some embodiments, the TGFβ related disease, disorder or condition is a disorder related to excessive cell proliferation, such as, for example, cancer. In certain embodiments, the TGFβ related disease or condition is characterized in expressing or over-expressing of TGFβ and/or TGFβ related genes such as TGFB1, TGFB2, TGFB3 genes.

The term “anti-TGFβ antibody moiety” refers to an antibody that is capable of specific binding to TGFβ (e.g. TGFβ1, TGFβ2, TGFβ3), and forms a portion of the protein targeting both CD39 and TGFβ. The term “anti-human TGFβ antibody moiety” refers to an antibody that is capable of specific binding to human TGFβ, and forms a portion of the protein targeting both CD39 and human TGFβ.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “CD39-positive cell” as used herein refers to a cell (e.g. a phagocytic cell) that expresses CD39 on the surface of the cell.

The term “pathway” as used herein refers to a group of biochemical reactions that together can convert one compound into another compound in a step-wise process. A product of the first step in a pathway may be a substrate for the second step, and a product of the second step may be a substrate for the third, and so on. Components of the pathway comprise all substrates, cofactors, byproducts, intermediates, end-products, any enzymes in the pathway. Accordingly, the term “adenosine pathway” as used herein refers to the collection of biochemical pathways, any one of which involves adenosine, e.g. the production of adenosine or conversion of adenosine into other substances. The term “TGFβ signaling pathway” as used herein refers to the collection of biochemical pathways, any one of which involves TGFβ, e.g. the production of TGFβ or conversion of TGFβ into other substances.

The term “antagonist” as used herein refers to a molecule that inhibits the expression level or activity of a protein, polypeptide or peptide, thereby reducing the amount, formation, function, and/or downstream signaling of the protein, polypeptide or peptide. For example, “antagonist of CD39” of the present disclosure refers to a molecule that inhibits the expression level or activity of CD39, thereby reducing the amount, formation, function, and/or downstream signaling of CD39. For another example, “antagonist of TGFβ” of the present disclosure refers to a molecule that inhibits the expression level or activity of TGFβ, thereby reducing the amount, formation, function, and/or downstream signaling of TGFβ.

The term “encoded” or “encoding” as used herein means capable of transcription into mRNA and/or translation into a peptide or protein. The term “encoding sequence” or “gene” refers to a polynucleotide sequence encoding a peptide or protein. These two terms can be used interchangeably in the present disclosure. In some embodiments, the encoding sequence is a complementary DNA (cDNA) sequence that is reversely transcribed from a messenger RNA (mRNA). In some embodiments, the encoding sequence is mRNA.

The term “antisense nucleotide” as used herein refers to an oligomeric compound that is capable of undergoing hybridization to a target nucleic acid through hydrogen bonding. For example, “an antisense nucleotide that targets an encoding sequence of CD39” refers to a nucleotide that is capable of undergoing hybridization to the encoding sequence of CD39 or a portion thereof.

### Conjugate Molecules Targeting CD39 and TGFβ

A potential limitation of current immune checkpoint inhibitors (e.g. PD1 and CTLA-4) is a tumor microenvironment (“TME”) enriched with adenosine and TGFβ. The adenosine and TGFβ signaling in the localized microenvironment of tumor-infiltrating T cells could skew them toward Tregs and attenuate the activation of immune effector cells. The present inventors unexpectedly found that by simultaneously targeting CD39 and TGFβ by a novel conjugate molecule, a more immune-normalized TME and synergistic anti-tumor effects can be achieved due to the simultaneous blockade of adenosine pathway (through inhibition of CD39) and TGFβ signaling pathway (via TGFβ trap). Indeed, the present inventors demonstrated that a conjugate molecule simultaneously targeting CD39 and TGFβ of the present disclosure exhibited synergistic anti-tumor effect beyond what was observed with the monotherapies with TGFβ receptor or anti-CD39 antibody, especially in terms of T cell survival, cytokine production and Treg suppression.

In one aspect, the present disclosure provides a conjugate molecule comprising a CD39 inhibitory portion capable of interfering interaction between CD39 and its substrate, and a TGFβ inhibitory portion capable of interfering interaction between TGFβ and its receptor. The conjugate molecule may be a small molecule, a compound (natural or synthetic), a peptide, a polypeptide, a protein, an interfering RNA, messenger RNA, etc. In certain embodiments, the conjugate molecule is not a mixture of two or more different substances (i.e. the two or more different substances are just put together and are not chemically bonded). In certain embodiments, the conjugate molecule is a bifunctional molecule, which is capable of interfering interaction between CD39 and its substrate, and capable of interfering interaction between TGFβ and its receptor.

The adenosine pathway participates in the creation of an immune-tolerant tumor microenvironment by regulating the functions of immune and inflammatory cells, such as macrophages, dendritic cells, myeloid-derived suppressor cells, T cells and natural killer (NK) cells. The adenosine pathway also regulates cancer growth and dissemination by interfering with cancer cell proliferation, apoptosis and angiogenesis via adenosine receptors that are expressed on cancer cells and endothelial cells, respectively. Solid tumors express high levels of CD39 and CD73, as well as low levels of nucleoside transporters (NTs), ecto-adenosine deaminase and its cofactor CD26, which lead to an increase in adenosine signaling in the cancer environment. In certain embodiments, the CD39 inhibitory portion of the present disclosure is capable of interfering interaction between CD39 and ATP/ADP. In certain embodiments, the CD39 inhibitory portion of the conjugate molecule is especially useful in treating, preventing or alleviating cancers.

In certain embodiments, the CD39 inhibitory portion of the conjugate molecule is an antagonist of CD39 selected from a group consisting of a CD39-binding agent, an RNAi that targets an encoding sequence of CD39, an antisense nucleotide that targets an encoding sequence of CD39, and an agent that competes with CD39 to bind to its substrate.

A molecule is considered to inhibit the expression level or activity of CD39 if the molecule causes a significant reduction in the expression (either at the level of transcription or translation) level or activity of CD39. Similarly, a molecule is considered to inhibit the binding between CD39 and its substrate (e.g. ATP or ADP) if the molecule causes a significant reduction in the binding between CD39 and its substrate, which causes a significant reduction in downstream signaling and functions mediated by CD39. A reduction is considered significant, for example, if the reduction is at least about 10%, 20%, 30%, 40%, 50%, 60%, 70%, 80%, 90%, 91%, 92%, 93%, 94%, 95%, 96%, 97%, 98%, or 99%.

A CD39-binding agent (antagonist) can act in two ways. In some embodiments, a CD39-binding agent of the present disclosure can compete with CD39 to bind to its substrate and thereby interfering with, blocking or otherwise preventing the binding of CD39 to its substrate. This type of antagonist, which binds the substrate but does not trigger the expected signal transduction, is also known as a “competitive antagonist”. In other embodiments, a CD39-binding agent of the present disclosure can bind to and sequester CD39 with sufficient affinity and specificity to substantially interfere with, block or otherwise prevent binding of CD39 to its substrate. This type of antagonist is also known as a “neutralizing antagonist”, and can include, for example, an antibody or aptamer directed to CD39 which specifically binds to CD39.

In certain embodiments, the CD39-binding agent is selected from the group consisting of an antibody or an antigen-binding fragment thereof that specifically recognizes CD39, and a small molecule compound that binds to CD39.

The term “small molecule compound” as used herein means a low molecular weight compound that may serve as an enzyme substrate or regulator of biological processes. In general, a “small molecule compound” is a molecule that is less than about 5 kilodaltons (kD) in size. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, in accordance with the present disclosure, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

In certain embodiments, the TGFβ inhibitory portion of the conjugate molecule is capable of interfering interaction between TGFβ and TGFβ receptor. In certain embodiments, the interaction between TGFβ and a TGFβ receptor is blocked by an agent that may disrupt the signal transduction cascade within the TGFβ signaling pathway, and disrupt or prevent TGFβ or a TGFβ superfamily ligand from binding to its endogenous receptor. Exemplary assays that can be used to determine the inhibitory activity of a TGFβ signaling pathway inhibitor include, without limitation, electrophoretic mobility shift assays, antibody supershift assays, as well as TGFβ-inducible gene reporter assays, as described in WO 2006/012954, among others.

In certain embodiments, the TGFβ inhibitory portion of the conjugate molecule is an antagonist of TGFβ selected from a group consisting of a TGFβ-binding agent, an RNAi that targets an encoding sequence of TGFβ, an antisense nucleotide that targets an encoding sequence of TGFβ, and an agent that competes with TGFβ to bind to its receptor (e.g. TGFβRI, TGFβRII, or TGFβRIII).

A molecule is considered to inhibit the expression level or activity of TGFβ if the molecule causes a significant reduction in the expression (either at the level of transcription or translation) level or activity of TGFβ. Similarly, a molecule is considered to inhibit the binding between TGFβ and its receptor (e.g. TGFβRI, TGFβRII, or TGFβRIII) if the molecule causes a significant reduction in the binding between TGFβ and its receptor, which causes a significant reduction in downstream signaling and functions mediated by TGFβ. A reduction is considered significant, for example, if the reduction is at least about 10%, 20%, 30%, 40%, 50%, 60%, 70%, 80%, 90%, 91%, 92%, 93%, 94%, 95%, 96%, 97%, 98%, or 99%.

A TGFβ-binding agent (antagonist) can act in two ways. In some embodiments, a TGFβ-binding agent of the present disclosure can compete with TGFβ to bind to its receptor and thereby interfering with, blocking or otherwise preventing the binding of TGFβ to its receptor. This type of antagonist, which binds the receptor but does not trigger the expected signal transduction, is also known as a “competitive antagonist”. In other embodiments, a TGFβ-binding agent of the present disclosure can bind to and sequester TGFβ with sufficient affinity and specificity to substantially interfere with, block or otherwise prevent binding of TGFβ to its receptor. This type of antagonist is also known as a “neutralizing antagonist”, and can include, for example, an antibody or aptamer directed to TGFβ which specifically binds to TGFβ.

In certain embodiments, the TGFβ-binding agent is selected from the group consisting of an antibody or an antigen-binding fragment thereof that specifically recognizes TGFβ, and a small molecule compound that binds to TGFβ.

In certain embodiments, the conjugate molecule of the present disclosure is a fusion protein comprising a CD39-binding domain linked to a TGFβ-binding domain.

As used herein, the term “binding domain” refers to a moiety that has an ability to specifically bind to a target molecule or complex. The binding domain may comprise a small molecule, peptide, modified peptide (e.g. peptides having non-natural amino acid residues), polypeptide, protein, antibody or antigen-binding fragments thereof, ligand, nucleic acid, or any combination thereof. For example, the term “CD39-binding domain” refers to a moiety that has an ability to specifically bind to CD39 (e.g. human and/or mouse CD39); the term “TGFβ-binding domain” refers to a moiety that has an ability to specifically bind to one or more family members or isoforms of the TGFβ family (e.g. TGFβ1, TGFβ2, or TGFβ3). The “TGFβ-binding domain” may also be referred to as “TGFβ Trap” in the present disclosure. Accordingly, a protein comprising a CD39-binding domain linked to a TGFβ-binding domain of the present disclosure may also be referred to as “anti-CD39/TGFβ Trap” in the present disclosure.

In certain embodiments, the conjugate molecule of the present disclosure specifically binds to human TGFβ1, human TGFβ2, and/or human TGFβ3. In certain embodiments, the conjugate molecule of the present disclosure specifically binds to human TGFβ1 and mouse TGFβ1 with similar affinity. In certain embodiments, the conjugate molecule of the present disclosure specifically binds to human TGFβ1 at an EC₅₀ of no more than 3×10⁻¹¹ M (e.g. no more than 2×10⁻¹¹ M, no more than 1×10⁻¹¹ M, no more than 0.9×10⁻¹¹ M, no more than 0.8×10⁻¹¹ M, no more than 0.7×10⁻¹¹ M, no more than 0.6×10⁻¹¹ M, no more than 0.5×10⁻¹¹ M) as measured by ELISA assay. In certain embodiments, the conjugate molecule of the present disclosure is capable of blocking human TGFβ1 and TGFβRII binding at an IC 50 of no more than 4×10⁻¹⁰ m (e.g. no more than 3×10⁻¹⁰ M, no more than 2×10⁻¹⁰ M, no more than 1×10⁻¹⁰ M, no more than 0.5×10⁻¹⁰ M) as measured by blocking assay. In certain embodiments, the conjugate molecule of the present disclosure is capable of binding to human CD39 in a dose-dependent manner as measured by FACS assay. In certain embodiments, the conjugate molecule of the present disclosure is capable of simultaneously binding to CD39 and TGFβ as measured by ELISA assay or FACS assay. In certain embodiments, the conjugate molecule of the present disclosure is capable of inhibiting TGFβ signal at an IC₅₀ no more than 4×10⁻¹¹ M as measured by a TGF-β SMAD reporter assay. In certain embodiments, the conjugate molecule of the present disclosure is capable of inhibiting ATPase activity in a CD39 expressing cell at an IC₅₀ of no more than 7×10⁻¹⁰ M (e.g. no more than 6×10⁻¹⁰ M, no more than 5×10⁻¹⁰ no more than 4×10⁻¹⁰ M, no more than 3×10⁻¹⁰ M, no more than 2×10⁻¹⁰ M, no more than 1×10⁻¹⁰ M, no more than 0.5×10⁻¹⁰ M) as measured by ATPase activity assay. In certain embodiments, the conjugate molecule of the present disclosure is capable of specifically binding to human CD39 at a K_{D} value of no more than 4×10⁻¹⁰ M (e.g. no more than 3×10⁻¹⁰ M, no more than 2×10⁻¹⁰ M, no more than 1×10⁻¹⁰ M, or no more than 0.5×10⁻¹⁰ M) as measured by Octet assay. In certain embodiments, the conjugate molecule of the present disclosure is capable of specifically binding to human TGFβ1 at a K_{D} value of no more than 4×10⁻¹¹ M (e.g. no more than 3×10⁻¹¹ M, no more than 2×10⁻¹¹ M, no more than 1×10⁻¹¹ M, or no more than 0.5×10⁻¹¹ M) as measured by Octet assay. In certain embodiments, the conjugate molecule of the present disclosure is capable of recovering T cell function as measured by a Treg suppression assay. In certain embodiments, the conjugate molecule of the present disclosure is capable of inhibiting human T cell apoptosis in a dose-dependent way. In certain embodiments, the conjugate molecule of the present disclosure is capable of promoting human T cell survival and activation over stimulation. In certain embodiments, the conjugate molecule of the present disclosure is capable of blocking TGFβ induced Foxp3 expression on total T cells. In certain embodiments, the conjugate molecule of the present disclosure is capable of restoring ATP induced inhibition on human T cell proliferation.

### TGFβ-Binding Domain

In certain embodiments, the TGFβ-binding domain binds to human and/or mouse TGFβ. In certain embodiments, the TGFβ-binding domain is capable of antagonizing and/or inhibiting TGFβ signaling pathway. In certain embodiments, the TGFβ-binding domain is capable of antagonizing and/or inhibiting TGFβ. In the present disclosure, the TGFβ-binding domain can be any moiety that specifically binds to one or more family members or isoforms of TGFβ family. In certain embodiments, the TGFβ-binding domain comprises a protein that binds to TGFβ1 (e.g. human TGFβ1), TGFβ2 (e.g. human TGFβ2), and/or TGFβ3 (e.g. human TGFβ3), or a variant thereof that has similar or improved TGFβ binding affinity. In certain embodiments, the TGFβ-binding domain binds to TGFβ1 (e.g. human TGFβ1). In certain embodiments, the TGFβ-binding domain binds to TGFβ2 (e.g. human TGFβ2). In certain embodiments, the TGFβ-binding domain binds to TGFβ3 (e.g. human TGFβ3). In certain embodiments, the TGFβ-binding domain specifically binds to TGFβ1 (e.g. human TGFβ1) and TGFβ2 (e.g. human TGFβ2). In certain embodiments, the TGFβ-binding domain specifically binds to TGFβ1 (e.g. human TGFβ1) and TGFβ3 (e.g. human TGFβ3). In certain embodiments, the TGFβ-binding domain specifically binds to TGFβ2 (e.g. human TGFβ2) and TGFβ3 (e.g. human TGFβ3). In certain embodiments, the TGFβ-binding domain specifically binds to TGFβ1 (e.g. human TGFβ1), TGFβ2 (e.g. human TGFβ2), and TGFβ3 (e.g. human TGFβ3). A person skilled in the art would appreciate that a TGFβ-binding domain that binds to one family member or isoform of TGFβ family may be capable of binding to one or more other family members or isoforms of TGFβ family with similar or higher affinity.

The TGFβ-binding domain of the present disclosure may be an anti-TGFβ antibody moiety or antigen-binding fragments thereof. Exemplary anti-TGFβ antibody moieties include fresolimumab and metelimumab, as well as the anti-TGFβ antibody moieties or antigen-binding fragments thereof described in, for example, U.S. Pat. No. 7,494,651B2, U.S. Pat. No. 8,383,780B2, U.S. Pat. No. 8,012,482B2, WO2017141208A1, each of which is incorporated herein by reference in its entirety.

The TGFβ-binding domain of the present disclosure may also be a TGFβ receptor (e.g. TGFβRI, TGFβRII, TGFβRIII) or a fragment thereof. In certain embodiments, the TGFβ-binding domain comprises a soluble TGFβ receptor (e.g. a soluble human TGFβ receptor), or a fragment thereof. In certain embodiments, the TGFβ-binding domain comprises an extracellular domain (ECD) of a TGFβ receptor (e.g. a human TGFβ receptor). In certain embodiments, the TGFβ receptor is selected from the group consisting of TGFβ Receptor I (TGFβRI), TGFβ Receptor II (TGFβRII), TGFβ Receptor III (TGFβRIII), and any combination thereof. In certain embodiments, the TGFβ receptor is TGFβRI (e.g. human TGFβRI). In certain embodiments, the TGFβ receptor is TGFβRII (e.g. human TGFβRII). In certain embodiments, the TGFβ receptor is TGFβRIII (e.g. human TGFβRIII).

In certain embodiments, the TGFβ-binding domain comprises an ECD of TGFβRI (e.g. human TGFβRI), an ECD of TGFβRII (e.g. human TGFβRII), an ECD of TGFβRIII (e.g. human TGFβRIII), or any combination thereof. In certain embodiments, the TGFβ-binding domain comprises an ECD of TGFβRI (e.g. human TGFβRI). In certain embodiments, the TGFβ-binding domain comprises an ECD of TGFβRII (e.g. human TGFβRII). In certain embodiments, the TGFβ-binding domain comprises an ECD of TGFβRIII (e.g. human TGFβRIII). In certain embodiments, the TGFβ-binding domain comprises an ECD of TGFβRI (e.g. human TGFβRI) and an ECD of TGFβRII (e.g. human TGFβRII). In certain embodiments, the TGFβ-binding domain comprises an ECD of TGFβRI (e.g. human TGFβRI) and an ECD of TGFβRIII (e.g. human TGFβRIII). In certain embodiments, the TGFβ-binding domain comprises an ECD of TGFβRII (e.g. human TGFβRII) and an ECD of TGFβRIII (e.g. human TGFβRIII).

In certain embodiments, In certain embodiments, the ECD of the TGFβ receptor comprises or consists of an amino acid sequence of SEQ ID NO: 163, SEQ ID NO: 164, or SEQ ID NO: 165, or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity thereof yet retaining binding specificity to TGFβ.

In certain embodiments, the TGFβ-binding domain comprises two or more (e.g. three, four, five, six, seven, eight, nine, ten, etc.) ECDs of an TGFβ receptor. In certain embodiments, the two or more ECDs are derived from the same TGFβ receptor. For example, the two or more ECDs are derived from TGFβRI (e.g. human TGFβRI), and are also referred to as “TGFβRI ECD” or “TGFβRI ECDs” in the present disclosure. For another example, the two or more ECDs are derived from TGFβRII (e.g. human TGFβRII), and are also referred to as “TGFβRII ECD” or “TGFβRII ECDs” in the present disclosure. For yet another example, the two or more ECDs are derived from TGFβRIII (e.g. human TGFβRIII), and are also referred to as “TGFβRIII ECD” or “TGFβRIII ECDs” in the present disclosure. In certain embodiments, the amino acid sequences of the two or more ECDs are identical. In certain embodiments, the amino acid sequences of the two or more ECDs are different by no more than 10, 9, 8, 7, 6, 5, 4, 3, 2, 1 amino acid. In certain embodiments, the amino acid sequences of the two or more ECDs are different but have at least 80%, at least 85%, at least 90%, at least 91%, at least 92%, at least 93%, at least 94%, at least 95%, at least 96%, at least 97%, at least 98%, at least 99% sequence identity to each other. In certain embodiments, the amino acid sequences of the two or more ECDs are different but each has at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 163-165 yet retaining binding specificity to TGFβ.

In certain embodiments, the two or more ECDs are derived from at least two different TGFβ receptors. For example, the two or more (e.g. three, four, five, six, seven, eight, nine, ten, etc.) ECDs are derived from at least two (e.g. two, three) different TGFβ receptors selected from TGFβRI (e.g. human TGFβRI), TGFβRII (e.g. human TGFβRII), and TGFβRIII (e.g. human TGFβRIII). In certain embodiments, the two or more ECDs comprise a first ECD derived from TGFβRI (e.g. human TGFβRI) and a second ECD derived from TGFβRII (e.g. human TGFβRII). In certain embodiments, the two or more ECDs comprise a first ECD derived from TGFβRI (e.g. human TGFβRI) and a second ECD derived from TGFβRIII (e.g. human TGFβRIII). In certain embodiments, the two or more ECDs comprise a first ECD derived from TGFβRII (e.g. human TGFβRII) and a second ECD derived from TGFβRIII (e.g. human TGFβRIII).

In certain embodiments, the ability of the anti-CD39/TGFβ Trap in blocking TGFβ and TGFβ receptor interaction is increased with the increase of TGFβ receptor ECDs. For example, the anti-CD39/TGFβ Trap with four TGFβRII ECDs is more potent than the anti-CD39/TGFβ Trap with two TGFβRII ECDs in blocking the interaction between TGFβ and TGFβRII.

In certain embodiments, the two or more ECDs are operably linked in series. In certain embodiments, the two or more ECDs are covalently or noncovalently linked to each other. In certain embodiments, the two or more ECDs are directly linked to each other or linked to each other via a linker. In certain embodiments, the two or more ECDs are linked via a first linker.

The term “linker” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more polypeptides. A linker may or may not have a secondary structure. Linker sequences are known in the art, see, for example, Holliger et al., Proc. Natl. Acad. Sci. USA 90:6444-6448 (1993); Poljak et al., Structure 2:1121-1123 (1994).

In certain embodiments, the first linker is selected from the group consisting of a cleavable linker, a non-cleavable linker, a peptide linker, a flexible linker, a rigid linker, a helical linker, and a non-helical linker. Any suitable linkers known in the art can be used. In certain embodiments, the first linker comprises a peptide linker. For example, a useful linker in the present disclosure may be rich in glycine and serine residues. Examples include linkers having a single or repeated sequences comprising threonine/serine and glycine, such as TGGGG (SEQ ID NO: 172), GGGGS (SEQ ID NO: 173) or SGGGG (SEQ ID NO: 174) or its tandem repeats (e.g. 2, 3, 4, 5, 6, 7, 8, 9, 10 or more repeats). In certain embodiments, the first linker used in the present disclosure comprises GGGGSGGGGSGGGGS (SEQ ID NO: 175). Alternatively, a linker may be a long peptide chain containing one or more sequential or tandem repeats of the amino acid sequence of GAPGGGGGAAAAAGGGGG (SEQ ID NO: 176). In certain embodiment, the first linker comprises 1, 2, 3, 4, 5, 6, 7, 8, 9, 10 or more sequential or tandem repeats of SEQ ID NO: 176. In certain embodiments, the peptide linker comprises a GS linker. In certain embodiments, the GS linker comprises one or more repeats of GGGS (SEQ ID NO: 177) or SEQ ID NO: 173. In certain embodiments, the peptide linker comprises an amino acid sequence of GGGGSGGGGSGGGGSG (SEQ ID NO: 182). In certain embodiments, the first linker comprises or consists of an amino acid sequence having at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 172-177, 182. The description of the first linker above is applicable to the first linker below.

In certain embodiments, the TGFβ-binding domain comprises an amino acid sequence selected from the group consisting of SEQ ID NO: 166, SEQ ID NO: 167, SEQ ID NO: 168, SEQ ID NO: 169, SEQ ID NO: 170, SEQ ID NO: 171, or any combination thereof.

The amino acid sequences of several exemplary ECDs of TGFβ receptor(s) are shown in Table 30 below. The first linkers are underlined.

TABLE 30
Amino Acid Sequences of Exemplary ECDs of TGFB Receptor

| Name | Amino Acid Sequence | SEQ ID NO |
|---|---|---|
| TGFβRI ECD | LQCFCHLCTKDNFTCVTDGLCFVSVTETTDKVIHNSM | 163 |
| | CIAEIDLIPRDRPFVCAPSSKTGSVTTTYCCNQDHCN | |
| | KIELPTTVKSSPGLGPVEL | |
| TGFβRII ECD | IPPHVQKSVNNDMIVTDNNGAVKFPQLCKFCDVREST | 164 |
| | CDNQKSCMSNCSITSICEKPQEVCVAVWRKNDENITL | |
| | ETVCHDPKLPYHDFILEDAASPKCIMKEKKKPGETFF | |
| | MCSCSSDECNDNIIFSEEYNTSNPD | |
| TGFβRIII ECD | GPEPGALCELSPVSASHPVQALMESFTVLSGCASRGT | 165 |
| | TGLPQEVHVLNLRTAGQGPGQLQREVTLHLNPISSVH | |
| | IHHKSVVELLNSPHPLVWHLKTERLATGVSRLFLVSE | |
| | GSVVQFSSANESLTAETEERNFPHGNEHLLNWARKEY | |
| | GAVTSFTELKIARNIYIKVGEDQVFPPKCNIGKNELS | |
| | LNYLAEYLQPKAAEGCVMSSQPQNEEVHIIELITPNS | |
| | NPYSAFQVDITIDIRPSQEDLEVVKNLILILKCKKSV | |
| | NWVIKSFDVKGSLKIIAPNSIGFGKESERSMTMTKSI | |
| | RDDIPSTQGNLVKWALDNGYSPITSYTMAPVANREHL | |
| | RLENNAEEMGDEEVHTIPPELRILLDPGALPALQNPP | |
| | IRGGEGQNGGLPFPFPDISRRVWNEEGEDGLPRPKDP | |
| | VIPSIQLFPGLREPEEVQGSVDIALSVKCDNEKMIVA | |
| | VEKDSFQASGYSGMDVTLLDPTCKAKMNGTHFVLESP | |
| | LNGCGTRPRWSALDGVVYYNSIVIQVPALGDSSGWPD | |
| | GYEDLESGDNGFPGDMDEGDASLFTRPEIVVENCSLQ | |
| | QVRNPSSFQEQPHGNITENMELYNTDLFLVPSQGVES | |
| | VPENGHVYVEVSVTKAEQELGFAIQTCFISPYSNPDR | |
| | MSHYTIIENICPKDESVKFYSPKRVHFPIPQADMDKK | |
| | RFSFVFKPVENTSLLELQCELTLCTKMEKHPQKLPKC | |
| | VPPDEACTSLDASIIWAMMQNKKTFTKPLAVIHHEAE | |
| | SKEKGPSMKEPNPISPPIFHGLDTLTV | |
| TGFβRI-TGFβRI | LQCFCHLCTKDNFTCVTDGLCFVSVTETTDKVIHNSM | 166 |
| ECD | CIAEIDLIPRDRPFVCAPSSKTGSVTTTYCCNQDHCN | |
| | KIELPTTVKSSPGLGPVELGGGGSGGGGSGGGGSLQC | |
| | FCHLCTKDNFTCVTDGLCFVSVTETTDKVIHNSMCIA | |
| | EIDLIPRDRPFVCAPSSKTGSVTTTYCCNQDHCNKIE | |
| | LPTTVKSSPGLGPVEL | |
| TGFβRII-TGFβRII | IPPHVQKSVNNDMIVTDNNGAVKFPQLCKFCDVREST | 167 |
| ECD | CDNQKSCMSNCSITSICEKPQEVCVAVWRKNDENITL | |
| | ETVCHDPKLPYHDFILEDAASPKCIMKEKKKPGETFF | |
| | MCSCSSDECNDNIIFSEEYNTSNPDGGGGSGGGGSGG | |
| | GGSIPPHVQKSVNNDMIVTDNNGAVKFPQLCKFCDVR | |
| | FSTCDNQKSCMSNCSITSICEKPQEVCVAVWRKNDEN | |
| | ITLETVCHDPKLPYHDFILEDAASPKCIMKEKKKPGE | |
| | TFFMCSCSSDECNDNIIFSEEYNTSNPD | |
| TGFβRIII- | GPEPGALCELSPVSASHPVQALMESFTVLSGCASRGT | 168 |
| TGFβRIII ECD | TGLPQEVHVLNLRTAGQGPGQLQREVTLHLNPISSVH | |
| | IHHKSVVFLLNSPHPLVWHLKTERLATGVSRLFLVSE | |
| | GSVVQESSANESLTAETEERNFPHGNEHLLNWARKEY | |
| | GAVTSFTELKIARNIYIKVGEDQVFPPKCNIGKNFLS | |
| | LNYLAEYLQPKAAEGCVMSSQPQNEEVHIIELITPNS | |
| | NPYSAFQVDITIDIRPSQEDLEVVKNLILILKCKKSV | |
| | NWVIKSFDVKGSLKIIAPNSIGFGKESERSMTMTKSI | |
| | RDDIPSTQGNLVKWALDNGYSPITSYTMAPVANRFHL | |
| | RLENNAEEMGDEEVHTIPPELRILLDPGALPALQNPP | |
| | IRGGEGQNGGLPFPFPDISRRVWNEEGEDGLPRPKDP | |
| | VIPSIQLFPGLREPEEVQGSVDIALSVKCDNEKMIVA | |
| | VEKDSFQASGYSGMDVTLLDPTCKAKMNGTHFVLESP | |
| | LNGCGTRPRWSALDGVVYYNSIVIQVPALGDSSGWPD | |
| | GYEDLESGDNGFPGDMDEGDASLFTRPEIVVENCSLQ | |
| | QVRNPSSFQEQPHGNITENMELYNTDLFLVPSQGVES | |
| | VPENGHVYVEVSVTKAEQELGFAIQTCFISPYSNPDR | |
| | MSHYTIIENICPKDESVKFYSPKRVHFPIPQADMDKK | |
| | RFSFVFKPVENTSLLFLQCELTLCTKMEKHPQKLPKC | |
| | VPPDEACTSLDASIIWAMMQNKKTFTKPLAVIHHEAE | |
| | SKEKGPSMKEPNPISPPIFHGLDTLTVGGGGSGGGGS | |
| | GGGGSGPEPGALCELSPVSASHPVQALMESFTVLSGC | |
| | ASRGTTGLPQEVHVLNLRTAGQGPGQLQREVTLHLNP | |
| | ISSVHIHHKSVVFLLNSPHPLVWHLKTERLATGVSRL | |
| | FLVSEGSVVQESSANESLTAETEERNFPHGNEHLLNW | |
| | ARKEYGAVTSFTELKIARNIYIKVGEDQVFPPKCNIG | |
| | KNFLSLNYLAEYLQPKAAEGCVMSSQPQNEEVHIIEL | |
| | ITPNSNPYSAFQVDITIDIRPSQEDLEVVKNLILILK | |
| | CKKSVNWVIKSFDVKGSLKIIAPNSIGFGKESERSMT | |
| | MTKSIRDDIPSTQGNLVKWALDNGYSPITSYTMAPVA | |
| | NRFHLRLENNAEEMGDEEVHTIPPELRILLDPGALPA | |
| | LQNPPIRGGEGQNGGLPFPFPDISRRVWNEEGEDGLP | |
| | RPKDPVIPSIQLFPGLREPEEVQGSVDIALSVKCDNE | |
| | KMIVAVEKDSFQASGYSGMDVTLLDPTCKAKMNGTHE | |
| | VLESPLNGCGTRPRWSALDGVVYYNSIVIQVPALGDS | |
| | SGWPDGYEDLESGDNGFPGDMDEGDASLFTRPEIVVE | |
| | NCSLQQVRNPSSFQEQPHGNITENMELYNTDLFLVPS | |
| | QGVFSVPENGHVYVEVSVTKAEQELGFAIQTCFISPY | |
| | SNPDRMSHYTIIENICPKDESVKFYSPKRVHFPIPQA | |
| | DMDKKRFSFVFKPVENTSLLELQCELTLCTKMEKHPQ | |
| | KLPKCVPPDEACTSLDASIIWAMMQNKKTFTKPLAVI | |
| | HHEAESKEKGPSMKEPNPISPPIFHGLDTLTV | |
| TGFβRI-TGFßRII | LQCFCHLCTKDNFTCVTDGLCFVSVTETTDKVIHNSM | 169 |
| ECD | CIAEIDLIPRDRPFVCAPSSKTGSVTTTYCCNQDHCN | |
| | KIELPTTVKSSPGLGPVELGGGGSGGGGSGGGGSIPP | |
| | HVQKSVNNDMIVTDNNGAVKFPQLCKFCDVRESTCDN | |
| | QKSCMSNCSITSICEKPQEVCVAVWRKNDENITLETV | |
| | CHDPKLPYHDFILEDAASPKCIMKEKKKPGETFFMCS | |
| | CSSDECNDNIIFSEEYNTSNPD | |
| TGFβRI-TGFβRIII | LQCFCHLCTKDNFTCVTDGLCFVSVTETTDKVIHNSM | 170 |
| ECD | CIAEIDLIPRDRPFVCAPSSKTGSVTTTYCCNQDHCN | |
| | KIELPTTVKSSPGLGPVELGGGGSGGGGSGGGGSGPE | |
| | PGALCELSPVSASHPVQALMESFTVLSGCASRGTTGL | |
| | PQEVHVLNLRTAGQGPGQLQREVTLHLNPISSVHIHH | |
| | KSVVFLLNSPHPLVWHLKTERLATGVSRLFLVSEGSV | |
| | VQFSSANESLTAETEERNFPHGNEHLLNWARKEYGAV | |
| | TSFTELKIARNIYIKVGEDQVFPPKCNIGKNFLSLNY | |
| | LAEYLQPKAAEGCVMSSQPQNEEVHIIELITPNSNPY | |
| | SAFQVDITIDIRPSQEDLEVVKNLILILKCKKSVNWV | |
| | IKSFDVKGSLKIIAPNSIGFGKESERSMTMTKSIRDD | |
| | IPSTQGNLVKWALDNGYSPITSYTMAPVANRFHLRLE | |
| | NNAEEMGDEEVHTIPPELRILLDPGALPALQNPPIRG | |
| | GEGQNGGLPFPFPDISRRVWNEEGEDGLPRPKDPVIP | |
| | SIQLFPGLREPEEVQGSVDIALSVKCDNEKMIVAVEK | |
| | DSFQASGYSGMDVTLLDPTCKAKMNGTHFVLESPLNG | |
| | CGTRPRWSALDGVVYYNSIVIQVPALGDSSGWPDGYE | |
| | DLESGDNGFPGDMDEGDASLFTRPEIVVENCSLQQVR | |
| | NPSSFQEQPHGNITENMELYNTDLFLVPSQGVESVPE | |
| | NGHVYVEVSVTKAEQELGFAIQTCFISPYSNPDRMSH | |
| | YTIIENICPKDESVKFYSPKRVHFPIPQADMDKKRFS | |
| | FVFKPVENTSLLFLQCELTLCTKMEKHPQKLPKCVPP | |
| | DEACTSLDASIIWAMMQNKKTFTKPLAVIHHEAESKE | |
| | KGPSMKEPNPISPPIFHGLDTLTV | |
| TGFβRII- | IPPHVQKSVNNDMIVTDNNGAVKFPQLCKFCDVREST | 171 |
| TGFβRIII ECD | CDNQKSCMSNCSITSICEKPQEVCVAVWRKNDENITL | |
| | ETVCHDPKLPYHDFILEDAASPKCIMKEKKKPGETFF | |
| | MCSCSSDECNDNIIFSEEYNTSNPDGGGGSGGGGSGG | |
| | GGSGPEPGALCELSPVSASHPVQALMESFTVLSGCAS | |
| | RGTTGLPQEVHVLNLRTAGQGPGQLQREVTLHLNPIS | |
| | SVHIHHKSVVELLNSPHPLVWHLKTERLATGVSRLEL | |
| | VSEGSVVQFSSANESLTAETEERNFPHGNEHLLNWAR | |
| | KEYGAVTSFTELKIARNIYIKVGEDQVFPPKCNIGKN | |
| | FLSLNYLAEYLQPKAAEGCVMSSQPQNEEVHIIELIT | |
| | PNSNPYSAFQVDITIDIRPSQEDLEVVKNLILILKCK | |
| | KSVNWVIKSFDVKGSLKIIAPNSIGFGKESERSMTMT | |
| | KSIRDDIPSTQGNLVKWALDNGYSPITSYTMAPVANR | |
| | FHLRLENNAEEMGDEEVHTIPPELRILLDPGALPALQ | |
| | NPPIRGGEGQNGGLPFPFPDISRRVWNEEGEDGLPRP | |
| | KDPVIPSIQLFPGLREPEEVQGSVDIALSVKCDNEKM | |
| | IVAVEKDSFQASGYSGMDVTLLDPTCKAKMNGTHEVL | |
| | ESPLNGCGTRPRWSALDGVVYYNSIVIQVPALGDSSG | |
| | WPDGYEDLESGDNGFPGDMDEGDASLFTRPEIVVENC | |
| | SLQQVRNPSSFQEQPHGNITENMELYNTDLFLVPSQG | |
| | VFSVPENGHVYVEVSVTKAEQELGFAIQTCFISPYSN | |
| | PDRMSHYTIIENICPKDESVKFYSPKRVHFPIPQADM | |
| | DKKRFSFVFKPVENTSLLFLQCELTLCTKMEKHPQKL | |
| | PKCVPPDEACTSLDASIIWAMMQNKKTFTKPLAVIHH | |
| | EAESKEKGPSMKEPNPISPPIFHGLDTLTV | |

### CD39-Binding Domain

In certain embodiments, the CD39-binding domain of the present disclosure binds to CD39 (e.g. human CD39, cynomolgus CD39, or mouse CD39). In certain embodiments, the CD39-binding domain of the present disclosure binds to human CD39.

In certain embodiments, the CD39-binding domain of the present disclosure comprises an anti-CD39 antibody moiety. Exemplary anti-CD39 antibody moieties include the anti-CD39 antibodies or antigen-binding fragments thereof described in, for example, U.S. Ser. No. 10/556,959B2, US20200277394A1, EP3429692A1, WO2018065552A1, each of which is incorporated herein by reference in its entirety. In certain embodiments, exemplary anti-CD39 antibody moieties are disclosed in Section Anti-CD39 Antibody Moieties and Section Illustrative Anti-CD39 Antibody Moieties of the present disclosure.

In certain embodiments, the anti-CD39 antibody moiety comprises one or more CDRs. In certain embodiments, the anti-CD39 antibody moiety comprises one or more CDRs described in Section Illustrative Anti-CD39 Antibody Moieties of the present disclosure. In certain embodiments, the anti-CD39 antibody moiety comprises a heavy chain variable region (VH) and a light chain variable region (VL). In certain embodiments, the anti-CD39 antibody moiety comprises a VH and a VL of an anti-CD39 antibody as disclosed in Section Illustrative Anti-CD39 Antibody Moieties of the present disclosure.

In certain embodiments, the anti-CD39 antibody moiety further comprises a heavy chain constant domain appended to a carboxyl terminus of the heavy chain variable region. In certain embodiments, the heavy chain constant region is derived from the group consisting of IgA, IgD, IgE, IgG, and IgM. In certain embodiments, the heavy chain constant region is derived from human IgG1, IgG2, IgG3, IgG4, IgA1, IgA2 or IgM. In certain embodiments, the heavy chain constant region is derived from human IgG1 (SEQ ID NO: 178) or IgG4 (SEQ ID NO: 179). In certain embodiments, the anti-CD39 antibody moiety further comprises a light chain constant domain appended to a carboxyl terminus of the light chain variable region. In certain embodiments, the light chain constant region is derived from Kappa light chain or Lamda light chain. The amino acid sequences of the Kappa light chain constant region and Lamda light chain constant region are shown in SEQ ID NO: 180 and SEQ ID NO: 181, respectively. The amino acid sequences of several exemplary constant regions are shown in Table 31 below.

TABLE 31
Amino Acid Sequences of Exemplary Constant Regions

| | | SEQ |
|---|---|---|
| Name | Amino Acid Sequence | ID NO |
| human IgG1 | ASTKGPSVFPLAPSSKSTSGGTAALGCLVKDYFPEPVTVSWNSGA | 178 |
| constant region | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK | |
| human IgG4 | ASTKGPSVFPLAPCSRSTSESTAALGCLVKDYFPEPVTVSWNSGA | 179 |
| constant region | LTSGVHTFPAVLQSSGLYSLSSVVTVPSSSLGTKTYTCNVDHKPS | |
| | NTKVDKRVESKYGPPCPPCPAPEFLGGPSVFLFPPKPKDTLMISR | |
| | TPEVTCVVVDVSQEDPEVQFNWYVDGVEVHNAKTKPREEQFNSTY | |
| | RVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENN | |
| | YKTTPPVLDSDGSFFLYSRLTVDKSRWQEGNVESCSVMHEALHNH | |
| | YTQKSLSLSLGK | |
| Light chain | RTVAAPSVFIFPPSDEQLKSGTASVVCLLNNFYPREAKVQWKVDNAL | 180 |
| constant region | QSGNSQESVTEQDSKDSTYSLSSTLTLSKADYEKHKVYACEVTHQGL | |
| (kappa) | SSPVTKSENRGEC | |
| Light chain | GQPKAAPSVTLFPPSSEELQANKATLVCLISDFYPGAVTVAWKADSS | 181 |
| constant region | PVKAGVETTTPSKQSNNKYAASSYLSLTPEQWKSHRSYSCQVTHEGS | |
| (Lamda) | TVEKTVAPTECS | |

### Linkage Between the TGFβ-Binding Domain and the CD39-Binding Domain

In the present disclosure, the TGFβ-binding domain can be linked to any portion of the CD39-binding domain (e.g. the anti-CD39 antibody moiety). In certain embodiments, the TGFβ-binding domain is linked to the anti-CD39 antibody moiety at a position selected from the group consisting of: 1) amino terminus of the heavy chain variable region, 2) amino terminus of the light chain variable region, 3) carboxyl terminus of the heavy chain variable region; 4) carboxyl terminus of the light chain variable region; 5) carboxyl terminus of the heavy chain constant region; and 6) carboxyl terminus of the light chain constant region, of the anti-CD39 antibody moiety.

The TGFβ-binding domain can be linked (covalently or non-covalently) to any portion (e.g. amino terminus or carboxyl terminus of the immunoglobulin chain) of the anti-CD39 antibody moiety (e.g. directly or via a second linker). Covalent linkage can be a chemical linkage or a genetic linkage. In certain embodiments, the second linker is selected from the group consisting of a cleavable linker, a non-cleavable linker, a peptide linker, a flexible linker, a rigid linker, a helical linker, and a non-helical linker. Any suitable linkers known in the art can be used. In certain embodiments, the second linker comprises a peptide linker. For example, a useful linker in the present disclosure may be rich in glycine and serine residues. Examples include linkers having a single or repeated sequences composed of threonine/serine and glycine, such as such as TGGGG (SEQ ID NO: 172), GGGGS (SEQ ID NO: 173) or SGGGG (SEQ ID NO: 174) or its tandem repeats (e.g. 2, 3, 4, 5, 6, 7, 8, 9, 10 or more repeats). In certain embodiments, the second linker used in the present disclosure comprises GGGGSGGGGSGGGGS (SEQ ID NO: 175). Alternatively, a linker may be a long peptide chain containing one or more sequential or tandem repeats of the amino acid sequence of GAPGGGGGAAAAAGGGGG (SEQ ID NO: 176). In certain embodiment, the second linker comprises 1, 2, 3, 4, 5, 6, 7, 8, 9, 10 or more sequential or tandem repeats of SEQ ID NO: 176. In certain embodiments, the peptide linker comprises a GS linker. In certain embodiments, the GS linker comprises one or more repeats of GGGS (SEQ ID NO: 177) or SEQ ID NO: 173. In certain embodiments, the peptide linker comprises an amino acid sequence of GGGGSGGGGSGGGGSG (SEQ ID NO: 182). In certain embodiments, the second linker comprises or consists of an amino acid sequence having at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 172-177, 182. The description of the second linker above is applicable to the second linker below.

In certain embodiments, the TGFβ-binding domain is linked to the heavy chain variable region of the anti-CD39 antibody moiety. The TGFβ-binding domain can be linked to any portion of the heavy chain variable region, including the amino terminus (N-terminus) or the carboxyl terminus (C-terminus) amino acid residue of the heavy chain variable region of the anti-CD39 antibody moiety. In certain embodiments, the TGFβ-binding domain is linked to the amino terminus of the heavy chain variable region of the anti-CD39 antibody moiety (e.g. directly or via a second linker). In certain embodiments, the TGFβ-binding domain is linked to the carboxyl terminus of the heavy chain variable region of the anti-CD39 antibody moiety (e.g. directly or via a second linker).

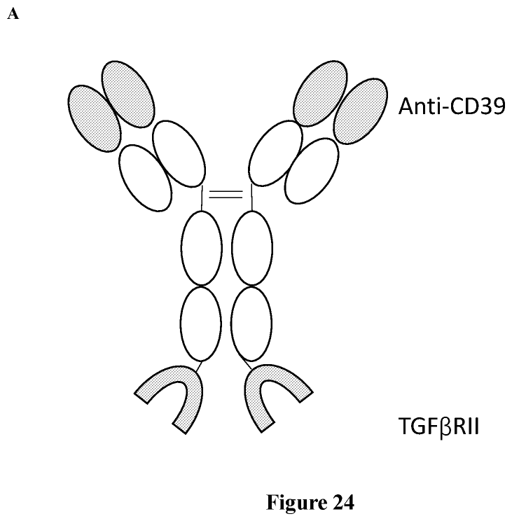

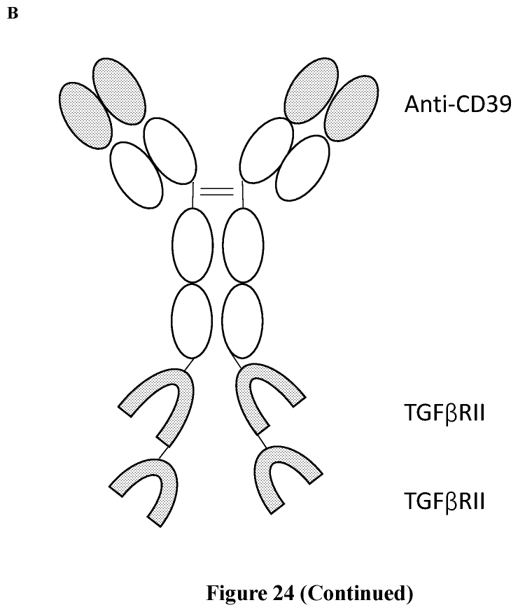

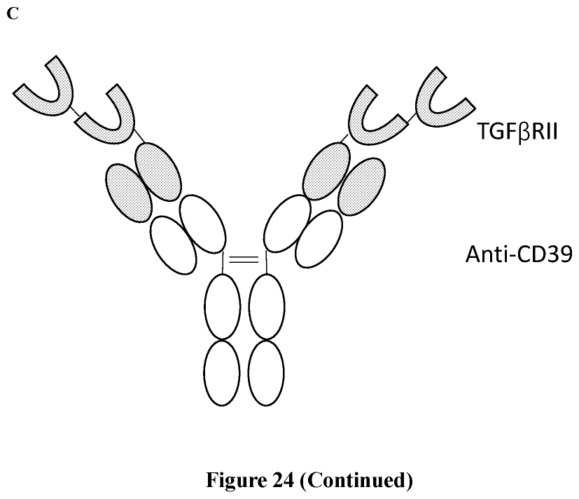

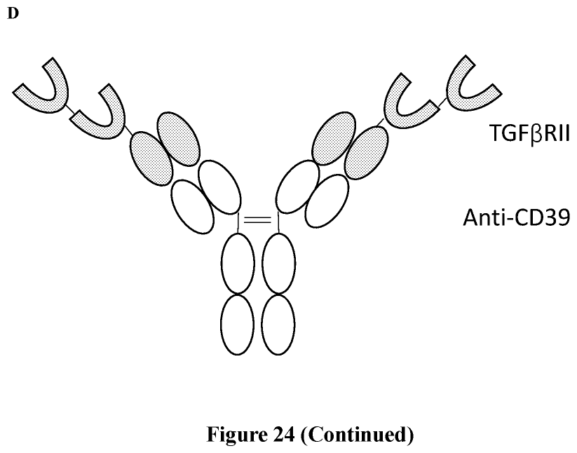

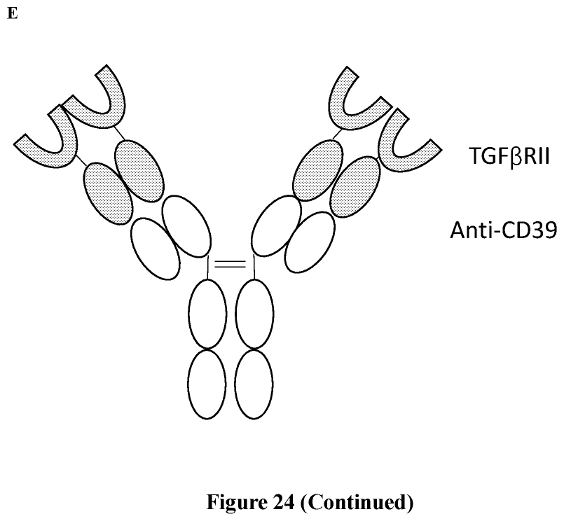

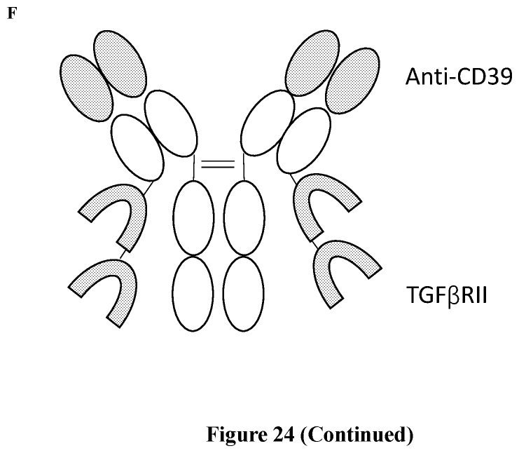

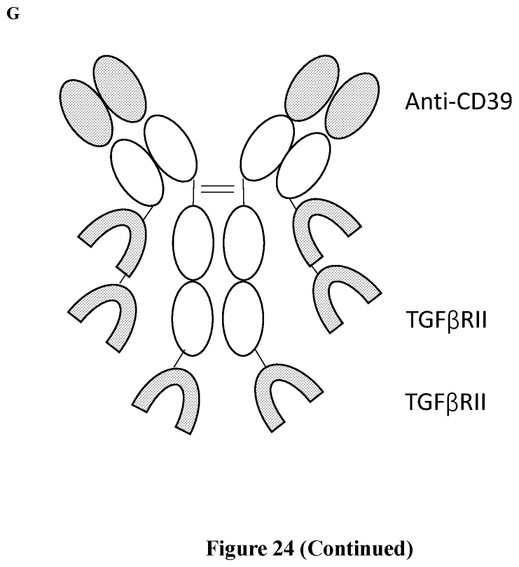

The schematic drawing of an exemplary anti-CD39/TGFβ Trap molecule comprising two TGFβRII ECDs linked to the amino terminus of each of the heavy chain variable region of the anti-CD39 antibody moiety is shown FIG. 24C of the present disclosure.

In certain embodiments, the TGFβ-binding domain is linked to the light chain variable region of the anti-CD39 antibody moiety. The TGFβ-binding domain can be linked to any portion of the light chain variable region, including the amino terminus or the carboxyl terminus amino acid residue of the light chain variable region of the anti-CD39 antibody moiety. In certain embodiments, the TGFβ-binding domain is linked to the amino terminus of the light chain variable region of the anti-CD39 antibody moiety (e.g. directly or via a second linker). In certain embodiments, the TGFβ-binding domain is linked to the carboxyl terminus of the light chain variable region of the anti-CD39 antibody moiety (e.g. directly or via a second linker).

The schematic drawing of an exemplary anti-CD39/TGFβ Trap molecule comprising two TGFβRII ECDs linked to the amino terminus of each of the light chain variable region of the anti-CD39 antibody moiety is shown FIG. 24D of the present disclosure.

In certain embodiments, the TGFβ-binding domain is linked to the heavy chain constant region of the anti-CD39 antibody moiety. The TGFβ-binding domain can be linked to any portion of the heavy chain constant region, including the amino terminus or the carboxyl terminus amino acid residue of the heavy chain constant region of the anti-CD39 antibody moiety. In certain embodiments, the TGFβ-binding domain is linked to the amino terminus of the heavy chain constant region of the anti-CD39 antibody moiety (e.g. directly or via a second linker). In certain embodiments, the TGFβ-binding domain is linked to the carboxyl terminus of the heavy chain constant region of the anti-CD39 antibody moiety (e.g. directly or via a second linker).

The schematic drawing of an exemplary anti-CD39/TGFβ Trap molecule comprising one TGFβRII ECD linked to the carboxyl terminus of each of the heavy chain constant region of the anti-CD39 antibody moiety is shown FIG. 24A of the present disclosure. The schematic drawing of an exemplary anti-CD39/TGFβ Trap molecule comprising two TGFβRII ECDs linked to the carboxyl terminus of each of the heavy chain constant region of the anti-CD39 antibody moiety is shown FIG. 24B of the present disclosure.

In certain embodiments, the TGFβ-binding domain is linked to the light chain constant region of the anti-CD39 antibody moiety. The TGFβ-binding domain can be linked to any portion of the light chain constant region, including the amino terminus or the carboxyl terminus amino acid residue of the light chain constant region of the anti-CD39 antibody moiety. In certain embodiments, the TGFβ-binding domain is linked to the amino terminus of the light chain constant region of the anti-CD39 antibody moiety (e.g. directly or via a second linker). In certain embodiments, the TGFβ-binding domain is linked to the carboxyl terminus of the light chain constant region of the anti-CD39 antibody moiety (e.g. directly or via a second linker).

The schematic drawing of an exemplary anti-CD39/TGFβ Trap molecule comprising two TGFβRII ECDs linked to the carboxyl terminus of each of the light chain constant region of the anti-CD39 antibody moiety is shown FIG. 24F of the present disclosure.

In certain embodiments, the protein of the present disclosure comprises two or more (e.g. three, four, five, six, seven, eight, nine, ten or more) TGFβ-binding domains which are all linked to the heavy chain variable region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the amino terminus of the heavy chain variable region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the carboxyl terminus of the heavy chain variable region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are linked to the amino terminus and the carboxyl terminus of the heavy chain variable region of the anti-CD39 antibody moiety (e.g. directly or via a second linker), respectively. In certain embodiments, the two or more TGFβ-binding domains are linked to each other directly or via a first linker.

In certain embodiments, the protein of the present disclosure comprises two or more (e.g. three, four, five, six, seven, eight, nine, ten or more) TGFβ-binding domains which are all linked to the light chain variable region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the amino terminus of the light chain variable region of the anti-CD39 antibody moiety (e.g. directly or via a second linker). In certain embodiments, the protein targeting both CD39 and TGFβ of the present disclosure comprises two or more (e.g. three, four, five, six, seven, eight, nine, ten or more) TGFβ-binding domains which are all linked to the carboxyl terminus of the light chain variable region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are linked to the amino terminus and the carboxyl terminus of the light chain variable region of the anti-CD39 antibody moiety (e.g. directly or via a second linker), respectively. In certain embodiments, the two or more TGFβ-binding domains are linked to each other directly or via a first linker.

In certain embodiments, the protein targeting both CD39 and TGFβ of the present disclosure comprises two or more (e.g. three, four, five, six, seven, eight, nine, ten or more) TGFβ-binding domains which are linked to the heavy and the light chain variable regions of anti-CD39 antibody moiety, respectively. In certain embodiments, the protein of the present disclosure comprises at least one (e.g. one, two, three, four, five, six, seven, eight, nine, ten or more) TGFβ-binding domain which is linked to the amino terminus of the heavy chain variable region of the anti-CD39 antibody moiety, and at least one (e.g. one, two, three, four, five, six, seven, eight, nine, ten or more) TGFβ-binding domain which is linked to the amino terminus of the light chain variable region of the anti-CD39 antibody moiety. In certain embodiments, the protein of the present disclosure comprises at least one (e.g. one, two, three, four, five, six, seven, eight, nine, ten or more) TGFβ-binding domain which is linked to the carboxyl terminus of the heavy chain variable region of the anti-CD39 antibody moiety, and at least one (e.g. one, two, three, four, five, six, seven, eight, nine, ten or more) TGFβ-binding domain which is linked to the carboxyl terminus of the light chain variable region of the anti-CD39 antibody moiety. In certain embodiments, the protein of the present disclosure comprises at least one (e.g. one, two, three, four, five, six, seven, eight, nine, ten or more) TGFβ-binding domain which is linked to the amino terminus of the heavy chain variable region of the anti-CD39 antibody moiety, and at least one (e.g. one, two, three, four, five, six, seven, eight, nine, ten or more) TGFβ-binding domain which is linked to the carboxyl terminus of the light chain variable region of the anti-CD39 antibody moiety. In certain embodiments, the protein of the present disclosure comprises at least one (e.g. one, two, three, four, five, six, seven, eight, nine, ten or more) TGFβ-binding domain which is linked to the carboxyl terminus of the heavy chain variable region of the anti-CD39 antibody moiety, and at least one (e.g. one, two, three, four, five, six, seven, eight, nine, ten or more) TGFβ-binding domain which is linked to the amino terminus of the light chain variable region of the anti-CD39 antibody moiety.

The schematic drawing of an exemplary anti-CD39/TGFβ Trap molecule comprising one TGFβRII ECD linked to the amino terminus of each of the heavy chain variable region of the anti-CD39 antibody moiety, and one TGFβRII ECD linked to the amino terminus of each of the light chain variable region of the anti-CD39 antibody moiety is shown in FIG. 24E of the present disclosure.

In certain embodiments, the protein targeting both CD39 and TGFβ of the present disclosure comprises two or more (e.g. three, four, five, six, seven, eight, nine, ten or more) TGFβ-binding domains which are all linked to the heavy chain constant region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the amino terminus of the heavy chain constant region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the carboxyl terminus of the heavy chain constant region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are linked to the amino terminus and the carboxyl terminus of the heavy chain constant region of the anti-CD39 antibody moiety (e.g. directly or via a second linker), respectively. In certain embodiments, the two or more TGFβ-binding domains are linked to each other directly or via a first linker.

In certain embodiments, the protein targeting both CD39 and TGFβ of the present disclosure comprises two or more (e.g. three, four, five, six, seven, eight, nine, ten or more) TGFβ-binding domains which are all linked to the light chain constant region of anti-CD39 antibody moiety (e.g. directly or via a second linker). In certain embodiments, the protein targeting both CD39 and TGFβ of the present disclosure comprises two or more (e.g. three, four, five, six, seven, eight, nine, ten or more) TGFβ-binding domains which are all linked to the amino terminus of the light chain constant region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are all linked to the carboxyl terminus of the light chain constant region of the anti-CD39 antibody moiety (e.g. directly or via a second linker). In certain embodiments, the protein of the present disclosure comprises two or more (e.g. three, four, five, six, seven, eight, nine, ten or more) TGFβ-binding domains which are linked to the amino terminus and the carboxyl terminus of the light chain constant region of the anti-CD39 antibody moiety (e.g. directly or via a second linker), respectively. In certain embodiments, the two or more TGFβ-binding domains are linked to each other directly or via a first linker.

In certain embodiments, the protein of the present disclosure comprises two or more TGFβ-binding domains which are linked to the heavy and the light chain constant regions of the anti-CD39 antibody moiety (e.g. directly or via a second linker), respectively. In certain embodiments, the protein of the present disclosure comprises at least one (e.g. one, two, three, four, five, six, seven, eight, nine, ten or more) TGFβ-binding domain which is linked to the amino terminus of the heavy chain constant region of the anti-CD39 antibody moiety, and at least one (e.g. one, two, three, four, five, six, seven, eight, nine, ten or more) TGFβ-binding domain which is linked to the amino terminus of the light chain constant region of the anti-CD39 antibody moiety. In certain embodiments, the protein of the present disclosure comprises at least one (e.g. one, two, three, four, five, six, seven, eight, nine, ten or more) TGFβ-binding domain which is linked to the carboxyl terminus of the heavy chain constant region of the anti-CD39 antibody moiety, and at least one (e.g. one, two, three, four, five, six, seven, eight, nine, ten or more) TGFβ-binding domain which is linked to the carboxyl terminus of the light chain constant region of the anti-CD39 antibody moiety. In certain embodiments, the protein of the present disclosure comprises at least one (e.g. one, two, three, four, five, six, seven, eight, nine, ten or more) TGFβ-binding domain which is linked to the amino terminus of the heavy chain constant region of the anti-CD39 antibody moiety, and at least one (e.g. one, two, three, four, five, six, seven, eight, nine, ten or more) TGFβ-binding domain which is linked to the carboxyl terminus of the light chain constant region of the anti-CD39 antibody moiety. In certain embodiments, the protein of the present disclosure comprises at least one (e.g. one, two, three, four, five, six, seven, eight, nine, ten or more) TGFβ-binding domain which is linked to the carboxyl terminus of the heavy chain constant region of the anti-CD39 antibody moiety, and at least one (e.g. one, two, three, four, five, six, seven, eight, nine, ten or more) TGFβ-binding domain which is linked to the amino terminus of the light chain constant region of the anti-CD39 antibody moiety.

The schematic drawing of an exemplary anti-CD39/TGFβ Trap molecule comprising one TGFβRII ECD linked to the carboxyl terminus of each of the heavy chain constant region of the anti-CD39 antibody moiety, and two TGFβRII ECDs linked to the carboxyl terminus of each of the light chain constant region of the anti-CD39 antibody moiety is shown FIG. 24G of the present disclosure.

In certain embodiments, the anti-CD39/TGFβ Trap molecule comprising TGFβ-binding domain(s) linked to the C-terminus of the heavy chain (e.g. the heavy chain variable region, the heavy chain constant region) or the light chain (e.g. the light chain variable region, the light chain constant region) of the anti-CD39 antibody moiety is more effective in binding to CD39 and/or TGFβ than the anti-CD39/TGFβ Trap molecule comprising TGFβ-binding domain(s) linked to the N-terminus of the heavy chain (e.g. the heavy chain variable region, the heavy chain constant region) or the light chain (e.g. the light chain variable region, the light chain constant region) of the anti-CD39 antibody moiety. In certain embodiments, the anti-CD39/TGFβ Trap molecule comprising TGFβ-binding domain(s) linked to the N-terminus of the heavy chain (e.g. the heavy chain variable region, the heavy chain constant region) or the light chain (e.g. the light chain variable region, the light chain constant region) of the anti-CD39 antibody moiety is more effective in binding to CD39 and/or TGFβ than the anti-CD39/TGFβ Trap molecule comprising TGFβ-binding domain(s) linked to the C-terminus of the heavy chain (e.g. the heavy chain variable region, the heavy chain constant region) or the light chain (e.g. the light chain variable region, the light chain constant region) of the anti-CD39 antibody moiety.

### Anti-CD39 Antibody Moieties

In certain embodiments, the CD39-binding domain of the conjugate molecules provided herein comprises an anti-CD39 antibody moiety or antigen-binding fragments thereof. In certain embodiments, the anti-CD39 antibody moieties and antigen-binding fragments thereof are capable of specifically binding to CD39.

In certain embodiments, the anti-CD39 antibody moieties and the antigen-binding fragments thereof provided herein specifically bind to human CD39 at an K_{D} value of no more than 10⁻⁷ M, no more than 8×10⁻⁸ M, no more than 5×10⁻⁸ M, no more than 2×10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M by Biacore assay. Biacore assay is based on surface plasmon resonance technology, see, for example, Murphy, M. et al., Current protocols in protein science, Chapter 19, unit 19.14, 2006. In certain embodiments, the K_{D} value is measured by the method as described in Example 5.1 of the present disclosure. In certain embodiments, the K_{D} value is measured at about 25° C., or at about 37° C. In certain embodiments, the antibodies and the antigen-binding fragments thereof provided herein have a K_{D} value measured at 25° C. comparable to that measured at 37° C., for example of about 80% to about 150%, of about 90% to about 130%, or of about 90% to about 120%, of about 90% to about 110% of that measured at 37° C.

In certain embodiments, the anti-CD39 antibody moieties and the antigen-binding fragments thereof provided herein specifically bind to human CD39 at an K_{D} value of no more than 10⁻⁸ M, no more than 8×10⁻⁹ M, no more than 5×10⁻⁹ M, no more than 4×10⁻⁹ M, no more than 3×10⁻⁹ M, no more than 2×10⁻⁹ M, no more than 1×10⁻⁹ M, no more than 9×10⁻¹⁰ no more than 8×10⁻¹⁰ M, no more than 7×10⁻¹⁰ M, or no more than 6×10⁻¹⁰ M by Octet assay. Octet assay is based on bio-layer interferometry technology, see, for example, Abdiche, Yasmina N., et al. Analytical biochemistry 386.2 (2009): 172-180, and Sun Y S., Instrumentation Science & Technology, 2014, 42(2): 109-127. In certain embodiments, the K_{D} value is measured by the method as described in Example 5.1 of the present disclosure.

Binding of the antibody moieties or the antigen-binding fragments thereof provided herein to human CD39 can also be represented by “half maximal effective concentration” (EC₅₀) value, which refers to the concentration of an antibody moiety where 50% of its maximal binding is observed. The EC₅₀ value can be measured by binding assays known in the art, for example, direct or indirect binding assay such as enzyme-linked immunosorbent assay (ELISA), FACS assay, and other binding assay. In certain embodiments, the antibody moieties and antigen-binding fragments thereof provided herein specifically bind to human CD39 at an EC₅₀ (i.e. 50% binding concentration) of no more than 10⁻⁷ M, no more than 8×10⁻⁸ M, no more than 5×10⁻⁸ M, no more than 2×10⁻⁸ M, no more than 10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M as measured by FACS (Fluorescence Activated Cell Sorting) assay. In certain embodiments, the binding is measured by ELISA or FACS assay.

In some embodiments, the anti-CD39 antibody moiety or an antigen-binding fragment thereof provided herein specifically binds to human CD39 (i.e. ENTPDase 1). In some embodiments, the anti-CD39 antibody moiety or an antigen-binding fragment thereof provided herein does not bind to other members of ENTPDase family. In some embodiments, the anti-CD39 antibody moiety or an antigen-binding fragment thereof provided herein specifically binds to human CD39, but does not specifically bind to ENTPDases 2, 3, 5, 6, for example, as measured by ELISA assay.

In certain embodiments, the anti-CD39 antibody moieties and antigen-binding fragments thereof provided herein specifically bind to human CD39 but not specifically bind to mouse CD39, for example, as measured by FACS assay.

In certain embodiments, the anti-CD39 antibody moieties and antigen-binding fragments thereof provided herein specifically bind to cynomolgus CD39 at an EC₅₀ of no more than 10⁻⁷ M, no more than 8×10⁻⁸ M, no more than 5×10⁻⁸ M, no more than 2×10⁻⁸ M, no more than 10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M by FACS assay.

In certain embodiments, the anti-CD39 antibody moieties and antigen-binding fragments thereof provided herein inhibit ATPase activity in a CD39 expressing cell at an IC 50 of no more than 50 nM, no more than 40 nM, no more than nM, no more than 20 nM, no more than 10 nM, no more than 8 nM, no more than nM, no more than 3 nM, no more than 1 nM, no more than 0.9 nM, no more than nM, no more than 0.7 nM, no more than 0.6 nM, no more than 0.5 nM, no more than 0.4 nM, no more than 0.3 nM, no more than 0.2 nM, no more than 0.1 nM, no more than 0.09 nM, no more than 0.08 nM, no more than 0.07 nM, no more than 0.06 nM, or no more than 0.05 nM as measured by ATPase activity assay. ATPase activity assay can be determined using any methods known in the art, for example by colorimetric detection of the phosphate released as a result of the ATPase activity. In certain embodiments, the ATPase activity is determined by the method as described in Example 3.3 of the present disclosure.

In certain embodiments, the anti-CD39 antibody moieties and antigen-binding fragments thereof provided herein are capable of enhancing ATP mediated monocytes activation at a concentration of no more than 50 nM (e.g., no more than no more than 30 nM, no more than 20 nM, no more than 10 nM, no more than no more than 3 nM, no more than 2 nM, no more than 1 nM, no more than 0.5 nM, or no more than 0.2 nM), as measured by analysis of CD80, CD86 and/or CD40 expression by FACS assay, where upregulation of CD80, CD86 and/or CD40 indicates monocytes activation. The activity of ATP mediated monocytes can be determined using methods known in the art, for example, by the method as described in Example 5.5 of the present disclosure.

In certain embodiments, the anti-CD39 antibody moieties and antigen-binding fragments thereof provided herein are capable of enhancing ATP mediated T cell activation in PBMC at a concentration of no more than 25 nM, no more than 20 nM, no more than 15 nM, no more than 10 nM, no more than 9 nM, no more than 8 nM, no more than 7 nM, no more than 6 nM, no more than 5 nM, no more than 4 nM, no more than 3 nM, no more than 2 nM, or no more than 1 nM, as measured by IL-2 secretion, or IFN-γ secretion, or CD4+ or CD8⁺ T cells proliferation, for example, by the method as described in Example 5.5 of the present disclosure.

In certain embodiments, the anti-CD39 antibody moieties and antigen-binding fragments thereof provided herein are capable of enhancing ATP mediated dendritic cell (DC) activation at a concentration of no more than 25 nM (or no more than 10 nM, or no more than 5 nM, or no more than 1 nM, or no more than 0.5 nM) as measured by analysis of CD83 expression by FACS assay.

In certain embodiments, the anti-CD39 antibody moieties and antigen-binding fragments thereof provided herein are capable of enhancing ATP mediated DC activation at a concentration of no more than 25 nM (or no more than 10 nM, or no more than 5 nM, or no more than 1 nM, or no more than 0.5 nM) as measured by the capability of the activated DC to promote T cell proliferation.

In certain embodiments, the anti-CD39 antibody moieties and antigen-binding fragments thereof provided herein are capable of enhancing ATP mediated DC activation at a concentration of no more than 25 nM (or no more than 10 nM, or no more than 5 nM, or no more than 1 nM, or no more than 0.5 nM) as measured by the capability of the activated DC to promote IFN-γ production in the mix-lymphocyte reaction (MLR) assay.

The activity of ATP mediated DC maturation can be determined using methods known in the art, for example the method as described in Example 5.5 of the present disclosure.

In certain embodiments, the anti-CD39 antibody moieties and antigen-binding fragments thereof provided herein are capable of blocking the inhibition of CD4⁺ T cell proliferation induced by adenosine (hydrolyzed from ATP) at a concentration of no more than 1 nM (e.g. no more than 0.1 nM, no more than 0.01 nM) as measured by FACS assay. T cell proliferation can be determined using methods known in the art, for example the method as described in Example 3.4 of the present disclosure.

In certain embodiments, the anti-CD39 antibody moieties and antigen-binding fragments thereof provided herein are capable of inhibiting tumor growth in a mammal in a NK cell or macrophage cell dependent manner.

In certain embodiments, the anti-CD39 antibody moieties and antigen-binding fragments thereof provided herein are capable of reversing human CD8⁺ T cell proliferation which was inhibited by eATP as measured by T cell proliferation, CD25⁺ cells, and living cells population. % T cell proliferation, % CD25⁺ cells, and % living cells can be determined using methods known in the art, for example the method as described in Example 3.4 of the present disclosure.

In certain embodiments, the anti-CD39 antibody moieties and antigen-binding fragments thereof provided herein are capable of enhancing human macrophage IL1β release induced by LPS stimulation at a concentration of no more than 50 nM (or no more than 12.5 nM, or no more than 3.13 nM, or no more than or no more than 0.2 nM, or no more than 0.049 nM, or no more than or no more than 0.003 nM, or no more than 0.0008 nM) as measured by ELISA assay. Macrophage IL-1β release can be determined using methods known in the art, for example the method as described in Example 5.5.4 of the present disclosure.

### Illustrative Anti-CD39 Antibody Moieties

In certain embodiments, the anti-CD39 antibody moieties (e.g. anti-human CD39 antibody moieties) and antigen-binding fragments thereof of the present disclosure comprise one or more (e.g. 1, 2, 3, 4, 5, or 6) CDRs comprising the sequences selected from the group consisting of NYGMN (SEQ ID NO: 1), KYWMN (SEQ ID NO: 2), NYWMN (SEQ ID NO: 3), DTFLH (SEQ ID NO: 4), DYNMY (SEQ ID NO: 5), DTYVH (SEQ ID NO: 6), LINTYTGEPTYADDFKD (SEQ ID NO: 7), EIRLKSNKYGTHYAESVKG (SEQ ID NO: 8), QIRLNPDNYATHX₁AESVKG (SEQ ID NO: 9), X₅₈IDPAX₅₉X₆₀NIKYDPKFQG (SEQ ID NO: 151), FIDPYNGYTSYNQKFKG (SEQ ID NO: 11), RIDPAIDNSKYDPKFQG (SEQ ID NO: 12), KGIYYDYVWFFDV (SEQ ID NO: 13), QLDLYWFFDV (SEQ ID NO: 14), HGX₂RGFAY (SEQ ID NO: 15), SPYYYGSGYRIFDV (SEQ ID NO: 16), IYGYDDAYYFDY (SEQ ID NO: 17), YYCALYDGYNVYAMDY (SEQ ID NO: 18), KASQDINRYIA (SEQ ID NO: 19), RASQSISDYLH (SEQ ID NO: 20), KSSQSLLDSDGRTHLN (SEQ ID NO: 21), SAFSSVNYMH (SEQ ID NO: 22), SATSSVSYMH (SEQ ID NO: 23), RSSKNLLHSNGITYLY (SEQ ID NO: 24), YTSTLLP (SEQ ID NO: 25), YASQSIS (SEQ ID NO: 26), LVSKLDS (SEQ ID NO: 27), TTSNLAS (SEQ ID NO: 28), STSNLAS (SEQ ID NO: 29), RASTLAS (SEQ ID NO: 30), LQYSNLLT (SEQ ID NO: 31), QNGHSLPLT (SEQ ID NO: 32), WQGTLFPWT (SEQ ID NO: 33), QQRSTYPFT (SEQ ID NO: 34), QQRITYPFT (SEQ ID NO: 35), and AQLLELPHT (SEQ ID NO: 36), wherein X₁ is Y or F, X₂ is S or T, X₅₈ is R or K, X₅₉ is N, G, S or Q, X₆₀ is G, A or D. In certain embodiments, the anti-CD39 antibody moieties and antigen binding fragments thereof of the present disclosure have no more than one, two or three amino acid residue substitutions to any of SEQ ID NOs: 1-9, 11-36, and 151.

Antibody “mAb13” as used herein refers to a monoclonal antibody comprising a heavy chain variable region having the sequence of SEQ ID NO: 42, and a light chain variable region having the sequence of SEQ ID NO: 51.

Antibody “mAb14” as used herein refers to a monoclonal antibody comprising a heavy chain variable region having the sequence of SEQ ID NO: 43, and a light chain variable region having the sequence of SEQ ID NO: 52.

Antibody “mAb19” as used herein refers to a monoclonal antibody comprising a heavy chain variable region having the sequence of SEQ ID NO: 44, and a light chain variable region having the sequence of SEQ ID NO: 53.

Antibody “mAb21” as used herein refers to a monoclonal antibody comprising a heavy chain variable region having the sequence of SEQ ID NO: 45, and a light chain variable region having the sequence of SEQ ID NO: 54.

Antibody “mAb23” as used herein refers to a monoclonal antibody comprising a heavy chain variable region having the sequence of SEQ ID NO: 47, and a light chain variable region having the sequence of SEQ ID NO: 56.

Antibody “mAb34” as used herein refers to a monoclonal antibody comprising a heavy chain variable region having the sequence of SEQ ID NO: 49, and a light chain variable region having the sequence of SEQ ID NO: 58.

Antibody “mAb35” as used herein refers to a monoclonal antibody comprising a heavy chain variable region having the sequence of SEQ ID NO: 50, and a light chain variable region having the sequence of SEQ ID NO: 59.

In certain embodiments, the anti-CD39 antibody moieties and antigen-binding fragments thereof of the present disclosure comprise one or more (e.g. 1, 2, 3, 4, 5, or 6) CDR sequences of Antibody mAb13, mAb14, mAb19, mAb21, mAb23, mAb34, or mAb35.

In certain embodiments, the anti-CD39 antibody moieties and antigen-binding fragments thereof of the present disclosure comprise HCDR1 comprising an amino acid sequence selected from the group consisting of SEQ ID NOs: 1-6, HCDR2 comprising an amino acid sequence selected from the group consisting of SEQ ID NOs: 7-9, 11-12, and 151, and HCDR3 comprising an amino acid sequence selected from the group consisting of SEQ ID NOs: 13-18, and/or LCDR1 comprising an amino acid sequence selected from the group consisting of SEQ ID NOs: 19-24, LCDR2 comprising an amino acid sequence selected from the group consisting of SEQ ID NOs: 25-30, and LCDR3 comprising an amino acid sequence selected from the group consisting of SEQ ID NOs: 31-36.

In certain embodiments, the anti-CD39 antibody moieties and antigen-binding fragments thereof of the present disclosure comprise a HCDR1 comprising the sequence of SEQ ID NO: 1, a HCDR2 comprising the sequence of SEQ ID NO: 7, a HCDR3 comprising the sequence of SEQ ID NO: 13, and/or a LCDR1 comprising the sequence of SEQ ID NO: 19, a LCDR2 comprising the sequence of SEQ ID NO: 25, and a LCDR3 comprising the sequence of SEQ ID NO: 31.

In certain embodiments, the anti-CD39 antibody moieties and antigen-binding fragments thereof of the present disclosure comprise a HCDR1 comprising the sequence of SEQ ID NO: 2, a HCDR2 comprising the sequence of SEQ ID NO: 8, a HCDR3 comprising the sequence of SEQ ID NO: 14, and/or a LCDR1 comprising the sequence of SEQ ID NO: 20, a LCDR2 comprising the sequence of SEQ ID NO: 26, and a LCDR3 comprising the sequence of SEQ ID NO: 32.

In certain embodiments, the anti-CD39 antibody moieties and antigen-binding fragments thereof of the present disclosure comprise a HCDR1 comprising the sequence of SEQ ID NO: 3, a HCDR2 comprising the sequence of SEQ ID NO: 37, a HCDR3 comprising the sequence of SEQ ID NO: 40, and/or a LCDR1 comprising the sequence of SEQ ID NO: 21, a LCDR2 comprising the sequence of SEQ ID NO: 27, and a LCDR3 comprising the sequence of SEQ ID NO: 33.

In certain embodiments, the anti-CD39 antibody moieties and antigen-binding fragments thereof of the present disclosure comprise a HCDR1 comprising the sequence of SEQ ID NO: 3, a HCDR2 comprising the sequence of SEQ ID NO: 38, a HCDR3 comprising the sequence of SEQ ID NO: 41, and/or a LCDR1 comprising the sequence of SEQ ID NO: 21, a LCDR2 comprising the sequence of SEQ ID NO: 27, and a LCDR3 comprising the sequence of SEQ ID NO: 33.

In certain embodiments, the anti-CD39 antibody moieties and antigen-binding fragments thereof of the present disclosure comprise a HCDR1 comprising the sequence of SEQ ID NO: 4, a HCDR2 comprising the sequence of SEQ ID NO: 10, a HCDR3 comprising the sequence of SEQ ID NO: 16, and/or a LCDR1 comprising the sequence of SEQ ID NO: 22, a LCDR2 comprising the sequence of SEQ ID NO: 28, and a LCDR3 comprising the sequence of SEQ ID NO: 34.

In certain embodiments, the anti-CD39 antibody moieties and antigen-binding fragments thereof of the present disclosure comprise a HCDR1 comprising the sequence of SEQ ID NO: 5, a HCDR2 comprising the sequence of SEQ ID NO: 11, a HCDR3 comprising the sequence of SEQ ID NO: 17, and/or a LCDR1 comprising the sequence of SEQ ID NO: 23, a LCDR2 comprising the sequence of SEQ ID NO: 29, and a LCDR3 comprising the sequence of SEQ ID NO: 35.

In certain embodiments, the anti-CD39 antibody moieties and antigen-binding fragments thereof of the present disclosure comprise a HCDR1 comprising the sequence of SEQ ID NO: 6, a HCDR2 comprising the sequence of SEQ ID NO: 12, a HCDR3 comprising the sequence of SEQ ID NO: 18, and/or a LCDR1 comprising the sequence of SEQ ID NO: 24, a LCDR2 comprising the sequence of SEQ ID NO: 30, and a LCDR3 comprising the sequence of SEQ ID NO: 36.

Table 1 below shows the CDR amino acid sequences of antibody moieties mAb13, mAb14, mAb19, mAb21, mAb23, mAb34, and mAb35. The CDR boundaries were defined or identified by the convention of Kabat. Table 2 below shows the heavy chain and light chain variable region amino acid sequences of antibody moieties mAb13, mAb14, mAb19, mAb21, mAb23, mAb34, and mAb35.

TABLE 1
CDR amino acid sequences of 7 monoclonal antibody moieties.

| Antibody | | | | |
|---|---|---|---|---|
| Moiety | | CDR1 | CDR2 | CDR3 |
| mAb13 | HCDR | SEQ ID NO: 1 | SEQ ID NO: 7 | SEQ ID NO: 13 |
| | | NYGMN | LINTYTGEPTYADD | KGIYYDYVWFF |
| | | | FKD | DV |
| | LCDR | SEQ ID NO: 19 | SEQ ID NO: 25 | SEQ ID NO: 31 |
| | | KASQDINRYIA | YTSTLLP | LQYSNLLT |
| mAb14 | HCDR | SEQ ID NO: 2 | SEQ ID NO: 8 | SEQ ID NO: 14 |
| | | KYWMN | EIRLKSNKYGTHYA | QLDLYWFFDV |
| | | | ESVKG | |
| | LCDR | SEQ ID NO: 20 | SEQ ID NO: 26 | SEQ ID NO: 32 |
| | | RASQSISDYLH | YASQSIS | QNGHSLPLT |
| mAb19 | HCDR | SEQ ID NO: 3 | SEQ ID NO: 37 | SEQ ID NO: 40 |
| | | NYWMN | QIRLNPDNYATHY | HGSRGFAY |
| | | | AESVKG | |
| | LCDR | SEQ ID NO: 21 | SEQ ID NO: 27 | SEQ ID NO: 33 |
| | | KSSQSLLDSDG | LVSKLDS | WQGTLFPWT |
| | | RTHLN | | |
| mAb21 | HCDR | SEQ ID NO: 3 | SEQ ID NO: 38 | SEQ ID NO: 41 |
| | | NYWMN | QIRLNPDNYATHFA | HGTRGFAY |
| | | | ESVKG | |
| | LCDR | SEQ ID NO: 21 | SEQ ID NO: 27 | SEQ ID NO: 33 |
| | | KSSQSLLDSDG | LVSKLDS | WQGTLFPWT |
| | | RTHLN | | |
| mAb23 | HCDR | SEQ ID NO: 4 | SEQ ID NO: 10 | SEQ ID NO: 16 |
| | | DTFLH | RIDPANGNIKYDPK | SPYYYGSGYRIF |
| | | | FQG | DV |
| | LCDR | SEQ ID NO: 22 | SEQ ID NO: 28 | SEQ ID NO: 34 |
| | | SAFSSVNYMH | TTSNLAS | QQRSTYPFT |
| mAb34 | HCDR | SEQ ID NO: 5 | SEQ ID NO: 11 | SEQ ID NO: 17 |
| | | DYNMY | FIDPYNGYTSYNQK | IYGYDDAYYFD |
| | | | FKG | Y |
| | LCDR | SEQ ID NO: 23 | SEQ ID NO: 29 | SEQ ID NO: 35 |
| | | SATSSVSYMH | STSNLAS | QQRITYPFT |
| mAb35 | HCDR | SEQ ID NO: 6 | SEQ ID NO: 12 | SEQ ID NO: 18 |
| | | DTYVH | RIDPAIDNSKYDPK | YYCALYDGYNV |
| | | | FQG | YAMDY |
| | LCDR | SEQ ID NO: 24 | SEQ ID NO: 30 | SEQ ID NO: 36 |
| | | RSSKNLLHSNG | RASTLAS | AQLLELPHT |
| | | ITYLY | | |

TABLE 2
Variable region amino acid sequences of 7 monoclonal antibody
moieties.

| Antibody | | |
|---|---|---|
| Moiety | VH | VL |
| mAb13 | SEQ ID NO: 42 | SEQ ID NO: 51 |
| | QIQLVQSGPELKKPGETVKISC | DIQMTQSPSSLSTSLGGKVSI |
| | KASGYTFTNYGMNWVKQAPG | TCKASQDINRYIAWYQHKPG |
| | KGLRWMGLINTYTGEPTYADD | KGPRLLIHYTSTLLPGIPSRFS |
| | FKDRFAFSLETSASTAFLQINNL | GSGSGRDYSFSISNLEPEDIA |
| | KDEDMATYFCARKGIYYDYV | TYFCLQYSNLLTFGGGTKLEI |
| | WFFDVWGAGTTVTVSS | K |
| mAb14 | SEQ ID NO: 43 | SEQ ID NO: 52 |
| | EVKLEESGGGLVQPGGSMKLS | DIVMTQSPAILSVTPGDRVSL |
| | CVASGFTFSKYWMNWVRQSPE | SCRASQSISDYLHWYQQKSH |
| | KGLEWVAEIRLKSNKYGTHYA | ESPRLLIKYASQSISGIPSRFS |
| | ESVKGRFTISRDDSKNNVYLQ | GSGSGSNFTLSINSVEPEDVG |
| | MNNLRPEDTGIYYCTTQLDLY | VYFCQNGHSLPLTFGAGTKL |
| | WFFDVWGAGTTVTVSS | ELR |
| mAb19 | SEQ ID NO: 44 | SEQ ID NO: 53 |
| | EVKLEKSGGGLVQPGGSMKLS | DVVMTQTPHTMSITIGQPASI |
| | CVASGFTFSNYWMNWVRQSPE | SCKSSQSLLDSDGRTHLNWL |
| | KGLEWVAQIRLNPDNYATHYA | FQRPGQSPKRLIYLVSKLDSG |
| | ESVKGRFTISRDDYKNSVYLQ | VPDRFTGSGSGTDFTLKISRV |
| | MSSLRAEDSGIYYCTQHGSRGF | EAEDLGVYYCWQGTLFPWT |
| | AYWGQGTLVTVS | FGGGTKLEIK |
| mAb21 | SEQ ID NO: 45 | SEQ ID NO: 54 |
| | EVKLEKSGGGLVQPGGSMKLS | DVVMTQTPLTLSITIGQPASIS |
| | CVASGFTFSNYWMNWVRQSPE | CKSSQSLLDSDGRTHLNWFF |
| | KGLEWVAQIRLNPDNYATHFAE | QRPGQSPKRLIYLVSKLDSG |
| | SVKGRFTISRDDSKNSVYLQM | VPDRFTGSGSGTDFTLKISRV |
| | NSLRAEDTGIYYCTEHGTRGFA | EAEDLGVYYCWQGTLFPWT |
| | YWGQGTLVTVSE | FGGGTKLEIK |
| mAb23 | SEQ ID NO: 47 | SEQ ID NO: 56 |
| | EVQLQQSGAELLRPGASVKLSC | QIVLTQSPAIMSASPGEKVTI |
| | TASGYNLKDTFLHWVKQRPEQ | TCSAFSSVNYMHWYQQKPG |
| | GLEWIGRIDPANGNIKYDPKFQ | TSPKLLIYTTSNLASGVPTRF |
| | GKATLTADTSSNTAYLQLISLTS | SGSGSGTSYSLTISRMEAEDA |
| | EDTAVYYCANSPYYYGSGYRIF | ATYYCQQRSTYPFTFGSGTK |
| | DVWGAGTTVTVSS | LEIQ |
| mAb34 | SEQ ID NO: 49 | SEQ ID NO: 58 |
| | EIQVQQSGPELVKPGASVKVSC | QIVLTQSPAIMSASPGEKVTI |
| | KASGYSFTDYNMYWVKQSHG | TCSATSSVSYMHWFRQKPGT |
| | KSLEWIGFIDPYNGYTSYNQKF | SPKLWIYSTSNLASGVPARFS |
| | KGKATLTIDKSSSTAFMHLNSLT | GSGSGTSYSLTISRMAAEDA |
| | SEDSAVYYCAIYGYDDAYYFD | ATYYCQQRITYPFTFGSGTK |
| | YWGQGTTLTVSS | LEIT |
| mAb35 | SEQ ID NO: 50 | SEQ ID NO: 59 |
| | EVRLQQSGAELVKPGASVKLS | DIVMTQAAFSNPVTLGTSASI |
| | CTASGFNIEDTYVHWMKQRPE | SCRSSKNLLHSNGITYLYWY |
| | QGLEWIGRIDPAIDNSKYDPKF | LQRPGQSPQLLIYRASTLASG |
| | QGKATITAVSSSNTAYLQLSSLT | VPNRFSGSESGTDFTLRISRV |
| | SEDTAVYYCALYDGYNVYAMD | EAEDVGVYYCAQLLELPHTF |
| | YWGQGTSVTVSS | GGGTKLEIK |

Given that each of antibody moieties mAb13, mAb14, mAb19, mAb21, mAb23, mAb34, and mAb35 can bind to CD39 and that antigen-binding specificity is provided primarily by the CDR1, CDR2 and CDR3 regions, the HCDR1, HCDR2 and HCDR3 sequences and LCDR1, LCDR2 and LCDR3 sequences of antibody moieties mAb13, mAb14, mAb19, mAb21, mAb23, mAb34, and mAb35 can be “mixed and matched” (i.e., CDRs from different antibody moieties can be mixed and matched, but each antibody moiety must contain a HCDR1, HCDR2 and HCDR3 and a LCDR1, LCDR2 and LCDR3) to create anti-CD39 binding molecules of the present disclosure. CD39 binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence (s). Likewise, when VL CDR sequences are mixed and matched, the LCDR1, LCDR2 and/or LCDR3 sequence from a particular VL sequence preferably is replaced with a structurally similar CDR sequence(s). For example, the HCDR1s of antibody moieties mAb13 and mAb19 share some structural similarity and therefore are amenable to mixing and matching. It will be readily apparent to a person skilled in the art that novel VH and VL sequences can be created by substituting one or more VH and/or VL CDR sequences with structurally similar sequences from the CDR sequences disclosed herein for monoclonal antibody moieties mAb13, mAb14, mAb19, mAb21, mAb23, mAb34, and mAb35.

CDRs are known to be responsible for antigen binding. However, it has been found that not all of the 6 CDRs are indispensable or unchangeable. In other words, it is possible to replace or change or modify one or more CDRs in anti-CD39 antibody moieties mAb13, mAb14, mAb19, mAb21, mAb23, mAb34, and mAb35, yet substantially retain the specific binding affinity to CD39.

In certain embodiments, the anti-CD39 antibody moieties and antigen-binding fragments thereof of the present disclosure comprise suitable framework region (FR) sequences, as long as the antibody moieties and antigen-binding fragments thereof can specifically bind to CD39. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the anti-CD39 antibody moieties and antigen-binding fragments thereof of the present disclosure are humanized. A humanized antibody moiety or antigen-binding fragment thereof is desirable in its reduced immunogenicity in human. A humanized antibody moiety is chimeric in its variable regions, as non-human CDR sequences are grafted to human or substantially human FR sequences. Humanization of an antibody moiety or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In some embodiments, the present disclosure provides 16 humanized antibody moieties of c14, which are designated as hu14.H1L1, hu14.H2L1, hu14.H3L1, hu14.H4L1, hu14.H1L2, hu14.H2L2, hu14.H3L2, hu14.H4L2, hu14.H1L3, hu14.H2L3, hu14.H3L3, hu14.H4L3, hu14.H1L4, hu14.H2L4, hu14.H3L4, and hu14.H4L4, respectively. The SEQ ID NOs of the heavy and light chain variable regions of each humanized antibody moiety of c14 are shown in Table 16 of Example 5.1. Each of the 16 humanized antibody moieties of c14 comprises a HCDR1 comprising the sequence of SEQ ID NO: 2, a HCDR2 comprising the sequence of SEQ ID NO: 8, a HCDR3 comprising the sequence of SEQ ID NO: 14, a LCDR1 comprising the sequence of SEQ ID NO: 20, a LCDR2 comprising the sequence of SEQ ID NO: 26, and a LCDR3 comprising the sequence of SEQ ID NO: 32. The CDR boundaries were defined or identified by the convention of Kabat.

In some embodiments, the present disclosure provides 31 humanized antibody moieties of c23, which are designated as hu23.H1L1, hu23.H2L1, hu23.H3L1, hu23.H4L1, hu23.H1L2, hu23.H2L2, hu23.H3L2, hu23.H4L2, hu23.H1L3, hu23.H2L3, hu23.H3L3, hu23.H4L3, hu23.H1L4, hu23.H2L4, hu23.H3L4, hu23.H4L4, hu23.H5L1, hu23.H6L1, hu23.H7L1, hu23.H1L5, hu23.H5L5, hu23.H6L5, hu23.H7L5, hu23.H1L6, hu23.H5L6, hu23.H6L6, hu23.H7L6, hu23.H1L7, hu23.H5L7, hu23.H6L7, and hu23.H7L7, respectively. The SEQ ID NOs of the heavy and light chain variable regions of each humanized antibody moiety of c23 are shown in Table 13 and Table 14 of Example 5.1. Each of the 31 humanized antibody moieties for antibody moiety c23 above comprises a HCDR1 comprising the sequence of SEQ ID NO: 4, a HCDR2 comprising the sequence of SEQ ID NO: 10, a HCDR3 comprising the sequence of SEQ ID NO: 16; a LCDR1 comprising the sequence of SEQ ID NO: 22, a LCDR2 comprising the sequence of SEQ ID NO: 28, and a LCDR3 comprising the sequence of SEQ ID NO: 34. The CDR boundaries were defined or identified by the convention of Kabat.

In some embodiments, the present disclosure also provides 6 humanized antibody moieties which have the same CDRs as c23 except that the amino acid sequences of HCDR2 are different. In some embodiments, the amino acid sequence of HCDR2 of the humanized antibody moieties of these c23 variants (c23′) comprises the amino acid sequence of X₅₈IDPAX₅₉X₆₀NIKYDPKFQG (SEQ ID NO: 151), wherein X₅₈ is R or K, X₅₉ is N, G, S or Q, X₆₀ is G, A or D. In some embodiments, the amino acid sequence of HCDR2 of the humanized antibody moieties of these c23 variants (c23′) comprises a sequence selected from the group consisting of RIDPAGGNIKYDPKFQG (SEQ ID NO: 134), RIDPASGNIKYDPKFQG (SEQ ID NO: 135), RIDPAQGNIKYDPKFQG (SEQ ID NO: 136), RIDPANANIKYDPKFQG (SEQ ID NO: 137), RIDPANDNIKYDPKFQG (SEQ ID NO: 138), and KIDPANGNIKYDPKFQG (SEQ ID NO: 139). The CDR boundaries were defined or identified by the convention of Kabat.

In some embodiments, the present disclosure also provided 4 humanized antibodies for c23 variants by yeast display, which are designated as hu23.201, hu23.203, hu23.207, and hu23.211. The heavy chain variable regions and light chain variable regions of humanized antibody moieties hu23.201, hu23.203, hu23.207, and hu23.211 are shown in Table 15 of Example 5.1. Each of the 4 humanized antibody moieties hu23.201, hu23.203, hu23.207, and hu23.211 comprises a HCDR1 comprising the sequence of SEQ ID NO: 4, a HCDR2 comprising the sequence of SEQ ID NO: 10, a HCDR3 comprising the sequence of SEQ ID NO: 16; a LCDR1 comprising the sequence of SEQ ID NO: 22, a LCDR2 comprising the sequence of SEQ ID NO: 28, and a LCDR3 comprising the sequence of SEQ ID NO: 34. The CDR boundaries were defined or identified by the convention of Kabat.

Table 3 below shows the 4 variants of humanized c14 heavy chain variable regions (i.e. hu14.VH_1, hu14.VH_2, hu14.VH_3, and hu14.VH_4) and 4 variants of humanized c14 light chain variable regions (i.e. hu14.VL_1, hu14.VL_2, hu14.VL_3, and hu14.VL_4). Table 4 below shows the amino acid sequences of the FR for the humanized c14 heavy chain and light chain variable regions. Table 5 below shows the FR amino acid sequences for each heavy and light chains of 16 humanized antibody moieties for chimeric antibody moiety c14, which are designated as hu14.H1L1, hu14.H2L1, hu14.H3L1, hu14.H4L1, hu14.H1L2, hu14.H2L2, hu14.H3L2, hu14.H4L2, hu14.H1L3, hu14.H2L3, hu14.H3L3, hu14.H4L3, hu14.H1L4, hu14.H2L4, hu14.H3L4, hu14.H4L4, respectively. The heavy chain variable regions and light chain variable regions of these 16 humanized antibody moieties are shown in Table 16 of Example 5.1.

TABLE 3
Amino acid sequences of the humanized variable regions for
humanized antibody moiety of c14.

| Antibody | | |
|---|---|---|
| Moiety | VH | VL |
| hu14.HIL1 | hu14.VH_1; SEQ ID NO: 68 | hu14.VL_1; SEQ ID NO: 69 |
| | EVQLVESGGGLVKPGGSLRLS | EIVLTQSPATLSLSPGERATLS |
| | CAASGFTFSKYWMNWVRQA | CRASQSISDYLHWYQQKPG |
| | PGKGLEWVGEIRLKSNKYGT | QAPRLLIYYASQSISGIPARFS |
| | HYAESVKGRFTISRDDSKNTL | GSGSGTDFTLTISSLEPEDFAV |
| | YLQMNSLKTEDTAVYYCTTQ | YYCQNGHSLPLTFGGGTKLE |
| | LDLYWFFDVWGQGTTVTVSS | IK |
| hu14.H2L2 | hu14.VH 2; SEQ ID NO: 70 | hu14 VL_2; SEQ ID NO: 71 |
| | EVQLVESGGGLVKPGGSLRLS | EIVLTQSPATLSLSPGERATLS |
| | CAASGFTFSKYWMNWVRQSP | CRASQSISDYLHWYQQKPG |
| | GKGLEWVGEIRLKSNKYGTH | QSPRLLIYYASQSISGIPARFS |
| | YAESVKGRFTISRDDSKNTLY | GSGSGTDFTLTISSLEPEDFAV |
| | LQMNSLKTEDTAVYYCTTQL | YFCQNGHSLPLTFGGGTKLEI |
| | DLYWFFDVWGQGTTVTVSS | K |
| hu14.H3L3 | hu14.VH_3; SEQ ID NO: 72 | hu14.VL_3; SEQ ID NO: 73 |
| | EVQLVESGGGLVKPGGSLRLS | EIVLTQSPATLSVSPGERATLS |
| | CAASGFTFSKYWMNWVRQSP | CRASQSISDYLHWYQQKPG |
| | GKGLEWVAEIRLKSNKYGTH | QSPRLLIYYASQSISGIPARFS |
| | YAESVKGRFTISRDDSKNTVY | GSGSGTDFTLTISSVEPEDFA |
| | LQMNSLKTEDTAVYYCTTQL | VYFCQNGHSLPLTFGGGTKL |
| | DLYWFFDVWGQGTTVTVSS | EIK |
| hu14.H4L4 | hu14.VH_4; SEQ ID NO: 74 | hu14.VL_4; SEQ ID NO: 75 |
| | EVQLVESGGGLVKPGGSMRL | EIVMTQSPATLSVSPGERVTL |
| | SCAASGFTFSKYWMNWVRQS | SCRASQSISDYLHWYQQKPG |
| | PGKGLEWVAEIRLKSNKYGT | QSPRLLIYYASQSISGIPARFS |
| | HYAESVKGRFTISRDDSKNTV | GSGSGTDFTLTISSVEPEDFA |
| | YLQMNSLKTEDTAVYYCTTQ | VYFCQNGHSLPLTFGGGTKL |
| | LDLYWFFDVWGQGTTVTVSS | EIK |

TABLE 4
Amino acid sequences of the humanized FR for
humanized antibody moiety of c14.

| SEQ ID NO. | Sequence |
|---|---|
| 79 | WGQGTTVTVSS |
| 98 | EIVLTQSPATLSLSPGERATLSC |
| 104 | WYQQKPGQAPRLLIY |
| 106 | GIPARFSGSGSGTDFTLTISSLEPEDFAVYYC |
| 119 | EVQLVESGGGLVKPGGSLRLSCAASGFTFS |
| 120 | EVQLVESGGGLVKPGGSMRLSCAASGFTFS |
| 121 | WVRQAPGKGLEWVG |
| 122 | WVRQSPGKGLEWVG |
| 123 | WVRQSPGKGLEWVA |
| 124 | RFTISRDDSKNTLYLQMNSLKTEDTAVYYCTT |
| 125 | RFTISRDDSKNTVYLQMNSLKTEDTAVYYCTT |
| 127 | EIVLTQSPATLSVSPGERATLSC |
| 128 | EIVMTQSPATLSVSPGERVTLSC |
| 130 | WYQQKPGQSPRLLIY |
| 132 | GIPARFSGSGSGTDFTLTISSLEPEDFAVYFC |
| 133 | GIPARFSGSGSGTDFTLTISSVEPEDFAVYFC |
| 153 | FGGGTKLEIK |

TABLE 5
The FR amino acid sequences for each humanized heavy and light
chain variable regions for humanized antibody moiety of c14.

| | FR1 | FR2 | FR3 | FR4 |
|---|---|---|---|---|
| VH or VL | (SEQ | (SEQ | (SEQ | (SEQ |
| Name | ID NO.) | ID NO.) | ID NO.) | ID NO.) |
| hu14.VH_1 | 119 | 121 | 124 | 79 |
| hu14.VH_2 | 119 | 122 | 124 | 79 |
| hu14.VH_3 | 119 | 123 | 125 | 79 |
| hu14.VH_4 | 120 | 123 | 125 | 79 |
| hu14.VL_1 | 98 | 104 | 106 | 153 |
| hu14.VL_2 | 98 | 130 | 132 | 153 |
| hu14.VL_3 | 127 | 130 | 133 | 153 |
| hu14.VL_4 | 128 | 130 | 133 | 153 |

Table 6 below shows the 7 variants of humanized c23 heavy chain variable regions (i.e. hu23.VH_1, hu23.VH_2, hu23.VH_3, hu23.VH_4, hu23.VH_5, hu23.VH_6, and hu23.VH_7) and 7 variants of humanized c23 light chain variable regions (i.e. hu23.VL_1, hu23.VL_2, hu23.VL_3, hu23.VL_4, hu23.VL_5, hu23.VL_6, and hu23.VL_7). Table 7 below shows the heavy and light chain variable region amino acid sequences of 4 humanized antibody moieties for chimeric antibody moiety c23 obtained by yeast display. Table 8 below shows the FR amino acid sequences of 35 humanized antibody moieties of c23. Table 9 below shows the FR amino acid sequences for each heavy and light chains of 35 humanized antibody moieties of c23.

TABLE 6
Amino acid sequences of the variable regions for humanized antibody
moiety of c23.

| Antibody | | |
|---|---|---|
| Moiety | VH | VL |
| hu23.H1L1 | hu23.VH_1, SEQ ID NO: 60 | hu23.VL 1, SEQ ID NO: 61 |
| | QVQLVQSGAEVKKPGASVKV | EIVLTQSPATLSLSPGERATLS |
| | SCKASGYNLKDTFLHWVRQA | CSAFSSVNYMHWYQQKPGQ |
| | PGQRLEWMGRIDPANGNIKY | APRLLIYTTSNLASGIPARFS |
| | DPKFQGRVTITRDTSASTAYM | GSGSGTDFTLTISSLEPEDFAV |
| | ELSSLRSEDTAVYYCARSPYY | YYCQQRSTYPFTFGQGTKLE |
| | YGSGYRIFDVWGQGTTVTVS | IK |
| | S | |
| hu23.H2L2 | hu23.VH_2, SEQ ID NO: 62 | hu23.VL_2, SEQ ID NO: 63 |
| | QVQLVQSGAEVKKPGASVKV | EIVLTQSPATLSLSPGERATLS |
| | SCKASGYNLKDTFLHWVRQA | CSAFSSVNYMHWYQQKPGQ |
| | PGQGLEWMGRIDPANGNIKY | APRLLIYTTSNLASGIPARFS |
| | DPKFQGRVTITADTSASTAYM | GSGSGTDYTLTISSLEPEDFA |
| | ELSSLRSEDTAVYYCANSPYY | VYYCQQRSTYPFTFGQGTKL |
| | YGSGYRIFDVWGQGTTVTVS | EIK |
| | S | |
| hu23.H3L3 | hu23.VH_3, SEQ ID NO: 64 | hu23.VL_3, SEQ ID NO: 65 |
| | QVQLVQSGAEVKKPGASVKL | EIVLTQSPATLSASPGERATLS |
| | SCKASGYNLKDTFLHWVRQA | CSAFSSVNYMHWYQQKPGQ |
| | PGQGLEWIGRIDPANGNIKYD | APRLLIYTTSNLASGIPARFS |
| | PKFQGRATITADTSASTAYME | GSGSGTDYTLTISSMEPEDFA |
| | LSSLRSEDTAVYYCANSPYYY | VYYCQQRSTYPFTFGQGTKL |
| | GSGYRIFDVWGQGTTVTVSS | EIK |
| hu23.H4L4 | hu23.VH_4, SEQ ID NO: 66 | hu23.VL_4, SEQ ID NO: 67 |
| | QVQLVQSGAEVKKPGASVKL | EIVLTQSPATLSASPGERVTIS |
| | SCKASGYNLKDTFLHWVKQA | CSAFSSVNYMHWYQQKPGQ |
| | PGQGLEWIGRIDPANGNIKYD | APRLLIYTTSNLASGIPARFS |
| | PKFQGRATLTADTSASTAYLEL | GSGSGTDYTLTISSMEPEDFA |
| | SSLRSEDTAVYYCANSPYYYG | VYYCQQRSTYPFTFGQGTKL |
| | SGYRIFDVWGQGTTVTVSS | EIK |
| hu23.H5L5 | hu23.VH_5, SEQ ID NO: 140 | hu23.VL_5, SEQ ID NO: 143 |
| | QVQLVQSGAEVKKPGASVKV | QIVLTQSPATLSLSPGERATLS |
| | SCKASGYNLKDTFLHWVRQA | CSAFSSVNYMHWYQQKPGQ |
| | PGQGLEWMGRIDPANGNIKY | APRLLIYTTSNLASGIPTRES |
| | DPKFQGRVTITADTSANTAYM | GSGSGTSYTLTISSLEPEDFAV |
| | ELISLRSEDTAVYYCANSPYY | YYCQQRSTYPFTFGQGTKLE |
| | YGSGYRIFDVWGQGTTVTVS | IK |
| | S | |
| hu23.H6L6 | hu23.VH_6, SEQ ID NO: 141 | hu23.VL_6, SEQ ID NO: 144 |
| | EVQLVQSGAEVKKPGASVKL | QIVLTQSPATLSASPGERATLS |
| | SCKASGYNLKDTFLHWVRQA | CSAFSSVNYMHWYQQKPGQ |
| | PGQGLEWIGRIDPANGNIKYD | APKLLIYTTSNLASGVPTRFS |
| | PKFQGRATITADTSANTAYME | GSGSGTSYTLTISSMEPEDFA |
| | LISLRSEDTAVYYCANSPYYY | VYYCQQRSTYPFTFGQGTKL |
| | GSGYRIFDVWGQGTTVTVSS | EIK |
| hu23.H7L7 | hu23.VH_7, SEQ ID NO: 142 | hu23.VL_7, SEQ ID NO: 145 |
| | EVQLVQSGAEVKKPGASVKL | QIVLTQSPATLSASPGERVTIT |
| | SCKASGYNLKDTFLHWVKQA | CSAFSSVNYMHWYQQKPGQ |
| | PGQGLEWIGRIDPANGNIKYD | APKLLIYTTSNLASGVPTRFS |
| | PKFQGKATLTADTSANTAYLE | GSGSGTSYTLTISSMEPEDFA |
| | LISLRSEDTAVYYCANSPYYY | VYYCQQRSTYPFTFGQGTKL |
| | GSGYRIFDVWGQGTTVTVSS | EIK |

TABLE 7
Amino acid sequences of the humanized variable regions for
humanized antibody moiety of c23 obtained by yeast display.

| Antibody | | |
|---|---|---|
| Moiety | VH | VL |
| hu23.201 | SEQ ID NO: 146 | SEQ ID NO: 111 |
| | QVQLVQSGAEVKKPGASVKV | EIVLTQSPATLSLSPGERATLS |
| | SCKASGYTLKDTFLHWVRQA | CSAFSSVNYMHWYQQKPGQ |
| | PGQRLEWMGRIDPANGNIKY | SPRLLIYTTSNLASGIPARFSG |
| | DPKFQGRVTLTADTSSNTAYM | SGSGTDYTLTISSLEPEDFAV |
| | ELSSLRSEDTAVYYCANSPYY | YYCQQRSTYPFTFGQGTKLE |
| | YGSGYRIFDVWGQGTLVTVSS | IK |
| hu23.203 | SEQ ID NO: 146 | SEQ ID NO: 112 |
| | QVQLVQSGAEVKKPGASVKV | EIVLTQSPATLTLSPGERATLS |
| | SCKASGYTLKDTFLHWVRQA | CSAFSSVNYMHWYQQKPGQ |
| | PGQRLEWMGRIDPANGNIKY | APRLLIYTTSNLASGIPARFS |
| | DPKFQGRVTLTADTSSNTAYM | GSGSGTDYTLTISSLEPEDFA |
| | ELSSLRSEDTAVYYCANSPYY | VYYCQQRSTYPFTFGQGTKL |
| | YGSGYRIFDVWGQGTLVTVSS | EIK |
| hu23.207 | SEQ ID NO: 147 | SEQ ID NO: 111 |
| | EVQLVQSGAEVKKPGASVKV | EIVLTQSPATLSLSPGERATLS |
| | SCKASGYTLKDTFLHWVRQA | CSAFSSVNYMHWYQQKPGQ |
| | PGQRLEWMGKIDPANGNIKY | SPRLLIYTTSNLASGIPARFSG |
| | DPKFQGRVTLTADTSSNTAYM | SGSGTDYTLTISSLEPEDFAV |
| | ELSSLRSEDTAVYYCANSPYY | YYCQQRSTYPFTFGQGTKLE |
| | YGSGYRIFDVWGQGTLVTVSS | IK |
| hu23.211 | SEQ ID NO: 39 | SEQ ID NO: 63 |
| | EVQLVQSGAEVKKPGASVKV | EIVLTQSPATLSLSPGERATLS |
| | SCKASGYTLKDTFLHWVRQA | CSAFSSVNYMHWYQQKPGQ |
| | PGQRLEWMGRIDPANGNIKY | APRLLIYTTSNLASGIPARFS |
| | DPKFQGRVTITADTSSNTAYM | GSGSGTDYTLTISSLEPEDFA |
| | ELSSLRSEDTAVYYCANSPYY | VYYCQQRSTYPFTFGQGTKL |
| | YGSGYRIFDVWGQGTLVTVSS | EIK |

TABLE 8
Amino acid sequences of the humanized FR for
humanized antibody moiety of c23.

| SEQ ID NO. | Sequence |
|---|---|
| 79 | WGQGTTVTVSS |
| 83 | FGQGTKLEIK |
| 84 | QVQLVQSGAEVKKPGASVKVSCKASGYNLK |
| 85 | QVQLVQSGAEVKKPGASVKLSCKASGYNLK |
| 86 | EVQLVQSGAEVKKPGASVKLSCKASGYNLK |
| 87 | WVRQAPGQRLEWMG |
| 88 | WVRQAPGQGLEWMG |
| 89 | WVRQAPGQGLEWIG |
| 90 | WVKQAPGQGLEWIG |
| 91 | RVTITRDTSASTAYMELSSLRSEDTAVYYCAR |
| 92 | RVTITADTSASTAYMELSSLRSEDTAVYYCAN |
| 93 | RATITADTSASTAYMELSSLRSEDTAVYYCAN |
| 94 | RATLTADTSASTAYLELSSLRSEDTAVYYCAN |
| 95 | RVTITADTSANTAYMELISLRSEDTAVYYCAN |
| 96 | RATITADTSANTAYMELISLRSEDTAVYYCAN |
| 97 | KATLTADTSANTAYLELISLRSEDTAVYYCAN |
| 98 | EIVLTQSPATLSLSPGERATLSC |
| 99 | EIVLTQSPATLSASPGERATLSC |
| 100 | EIVLTQSPATLSASPGERVTISC |
| 101 | QIVLTQSPATLSLSPGERATLSC |
| 102 | QIVLTQSPATLSASPGERATLSC |
| 103 | QIVLTQSPATLSASPGERVTITC |
| 104 | WYQQKPGQAPRLLIY |
| 105 | WYQQKPGQAPKLLIY |
| 106 | GIPARFSGSGSGTDFTLTISSLEPEDFAVYYC |
| 107 | GIPARFSGSGSGTDYTLTISSLEPEDFAVYYC |
| 108 | GIPARFSGSGSGTDYTLTISSMEPEDFAVYYC |
| 109 | GIPTRFSGSGSGTSYTLTISSLEPEDFAVYYC |
| 110 | GVPTRFSGSGSGTSYTLTISSMEPEDFAVYYC |
| 115 | EVQLVQSGAEVKKPGASVKVSCKASGYTLK |
| 116 | RVTLTADTSSNTAYMELSSLRSEDTAVYYCAN |
| 117 | RVTITADTSSNTAYMELSSLRSEDTAVYYCAN |
| 118 | WGQGTLVTVSS |
| 129 | EIVLTQSPATLTLSPGERATLSC |
| 130 | WYQQKPGQSPRLLIY |
| 131 | QVQLVQSGAEVKKPGASVKVSCKASGYTLK |

TABLE 9
The FR amino acid sequences for each humanized heavy and light
chain variable regions for humanized antibody moiety of c23.

| | FR1 | FR2 | FR3 | FR4 |
|---|---|---|---|---|
| VH or VL | (SEQ | (SEQ | (SEQ | (SEQ |
| Name | ID NO.) | ID NO.) | ID NO.) | ID NO.) |
| hu23.VH_1 | 84 | 87 | 91 | 79 |
| hu23.VH_2 | 84 | 88 | 92 | 79 |
| hu23.VH_3 | 85 | 89 | 93 | 79 |
| hu23.VH_4 | 85 | 90 | 94 | 79 |
| hu23.VH_5 | 84 | 88 | 95 | 79 |
| hu23.VH_6 | 86 | 89 | 96 | 79 |
| hu23.VH_7 | 86 | 90 | 97 | 79 |
| hu23.VH_201 | 131 | 87 | 116 | 118 |
| hu23.VH_207 | 115 | 87 | 116 | 118 |
| hu23.VH_211 | 115 | 87 | 117 | 118 |
| hu23.VL_1 | 98 | 104 | 106 | 83 |
| hu23.VL_2 | 98 | 104 | 107 | 83 |
| hu23.VL_3 | 99 | 104 | 108 | 83 |
| hu23.VL_4 | 100 | 104 | 108 | 83 |
| hu23.VL_5 | 101 | 104 | 109 | 83 |
| hu23.VL_6 | 102 | 105 | 110 | 83 |
| hu23.VL_7 | 103 | 105 | 110 | 83 |
| hu23.VL_201 | 98 | 130 | 107 | 83 |
| hu23.VL_203 | 129 | 104 | 107 | 83 |
| hu23.VL_211 | 98 | 104 | 107 | 83 |

In certain embodiments, the humanized anti-CD39 antibody moieties or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody moiety or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody moiety or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the humanized anti-CD39 antibody moieties and antigen-binding fragments thereof of the present disclosure comprise a heavy chain HFR1 comprising the sequence of X₁₉VQLVX₂₀SGX₂₁X₂₂X₂₃X₂₄KPGX₂₅SX₂₆X₂₇X₂₈₅CX₂₉A₅GX₃₀X₃₁X₃₂X₃₃ (SEQ ID NO: 76) or a homologous sequence of at least 80% sequence identity thereof, a heavy chain HFR2 comprising the sequence of WVX₃₄QX₃₅PGX₃₆X₃₇LEWX₃₈X₃₉ (SEQ ID NO: 77) or a homologous sequence of at least 80% sequence identity thereof, a heavy chain HFR3 comprising the sequence of X₄₀X₄₁TX₄₂X₄₃X₄₄DX₄₅SX₄₆X₄₇TX₄₈YX₄₉X₅₀X₅₁X₅₂SLX₅₃X₅₄EDTAVYYCX₅₅X₅₆ (SEQ ID NO: 78) or a homologous sequence of at least 80% sequence identity thereof, and a heavy chain HFR4 comprising the sequence of WGQGTX₅₇VTVSS (SEQ ID NO: 126) or a homologous sequence of at least 80% sequence identity thereof, wherein X₁₉ is Q or E; X₂₀ is E or Q; X₂₁ is G or A; X₂₂ is G or E; X₂₃ is L or V; X₂₄ is V or K; X₂₅ is G or A; X₂₆ is L, M or V; X₂₇ is R or K; X₂₈ is V or L; X₂₉ is A or K; X₃₀ is F or Y; X₃₁ is N or T; X₃₂ is F or L; X₃₃ is S or K; X₃₄ is R or K; X₃₅ is A or S; X₃₆ is K or Q; X₃₇ is R or G; X₃₈ is M, I or V; X₃₉ is G or A; X₄₀ is R or K; X₄₁ is V, A or F; X₄₂ is I or L; X₄₃ is S or T; X₄₄ is R or A; X₄₅ is D or T; X₄₆ is K, A or S; X₄₇ is S or N; X₄₈ is L, V or A; X₄₉ is M or L; X₅₀ is Q or E; X₅₁ is M or L; X₅₂ is 5, I or N; X₅₃ is R or K; X₅₄ is S or T; X₅₅ is A or T; X₅₆ is R, N or T; and X₅₇ is T or L.

In certain embodiments, the humanized anti-CD39 antibody moieties and antigen-binding fragments thereof of the present disclosure comprise a light chain LFR1 comprising the sequence of X₃IVX₄TQSPATLX₅X₆SPGERX₇TX₈X₉C (SEQ ID NO: 80) or a homologous sequence of at least 80% sequence identity thereof, a light chain LFR2 comprising the sequence of WYQQKPGQX₁₀PX11LLIY (SEQ ID NO: 81) or a homologous sequence of at least 80% sequence identity thereof, a light chain LFR3 comprising the sequence of GX₁₂PXDRFSGSGSGTX₁₄X₁₅TLTISSX₁₆EPEDFAVYX₁₇C (SEQ ID NO: 82) or a homologous sequence of at least 80% sequence identity thereof, and a light chain LFR4 comprising the sequence of FGX₁₈GTKLEIK (SEQ ID NO: 152) or a homologous sequence of at least 80% sequence identity thereof, wherein X₃ is E or Q; X₄ is L or M; X₅ is S or T; X₆ is L, V or A; X₇ is A or V; X₈ is L or I; X₉ is S or T; X₁₀ is A or S; X₁₁ is R or K; X₁₂ is I or V; X₁₃ is A or T; X₁₄ is D or S; X₁₅ is F or Y; X₁₆ is L, M or V; X₁₇ is Y or F; X₁₈ is G or Q.

In certain embodiments, the humanized anti-CD39 antibody moieties and antigen-binding fragments thereof of the present disclosure comprise a heavy chain HFR1 comprising the sequence of EVQLVESGGGLVKPGGSX₆₁RLSCAASGFTFS (SEQ ID NO: 154), or a homologous sequence of at least 80% sequence identity thereof; a heavy chain HFR2 comprising the sequence of WVRQX₆₂PGKGLEWVX₆₃ (SEQ ID NO: 155) or a homologous sequence of at least 80% sequence identity thereof; a heavy chain HFR3 comprising the sequence of RFTISRDDSKNTX₆₄YLQMNSLKTEDTAVYYCTT (SEQ ID NO: 156), or a homologous sequence of at least 80% sequence identity thereof; a heavy chain HFR4 comprising the sequence of WGQGTTVTVSS (SEQ ID NO: 79), or a homologous sequence of at least 80% sequence identity thereof, wherein X₆₁ is L or M, X₆₂ is A or X₆₃ is G or A, X₆₄ is L or V.

In certain embodiments, the humanized anti-CD39 antibody moieties and antigen-binding fragments thereof of the present disclosure comprise a light chain LFR1 comprising the sequence of EIVX₆₅TQSPATLSX₆₆SPGERX₆₇TLSC (SEQ ID NO: 157), or a homologous sequence of at least 80% sequence identity thereof; a light chain LFR2 comprising the sequence of WYQQKPGQX₆₈PRLLIY (SEQ ID NO: 158), or a homologous sequence of at least 80% sequence identity thereof; a light chain LFR3 comprising the sequence of GIPARFSGSGSGTDFTLTISSX₆₉EPEDFAVYX₇₀C (SEQ ID NO: 159), or a homologous sequence of at least 80% sequence identity thereof, and a light chain LFR4 comprising the sequence of FGGGTKLEIK (SEQ ID NO: 153), or a homologous sequence of at least 80% sequence identity thereof, wherein X₆₅ is L or M; X₆₆ is L or V; X₆₇ is A or V; X₆₈ is A or S; X₆₉ is L or V; and X₇₀ is Y or F.

In certain embodiments, the humanized anti-CD39 antibody moieties and antigen-binding fragments thereof of the present disclosure comprise a heavy chain HFR1 comprising the sequence of X₇₁VQLVQSGAEVKKPGASVKX₇₂SCKASGYX₇₃LK (SEQ ID NO: 160), or a homologous sequence of at least 80% sequence identity thereof; a heavy chain HFR2 comprising the sequence of WVX₇₄QAPGQX₇₅LEWX₇₆G (SEQ ID NO: 161) or a homologous sequence of at least 80% sequence identity thereof; a heavy chain HFR3 comprising the sequence of X₇₇X₇₈TX₇₉TX₈₀DTSX₈₁X₈₂TAYX₈₃ELX₈₄SLRSEDTAVYYCAX₈₅ (SEQ ID NO: 149), or a homologous sequence of at least 80% sequence identity thereof; a heavy chain HFR4 comprising the sequence of WGQGTX₅₇VTVSS (SEQ ID NO: 126), or a homologous sequence of at least 80% sequence identity thereof, wherein X₅₇ is as defined above, X₇₁ is Q or E; X₇₂ is V or L; X₇₃ is N or T; X₇₄ is R or K; X₇₅ is R or G; X₇₆ is M or I; X₇₇ is R or K; X₇₈ is V or A; X₇₉ is I or L; X₈₀ is R or A; X₈₁ is A or S; X₈₂ is S or N; X₈₃ is M or L; X₈₄ is S or I; X₈₅ is R or N.

In certain embodiments, the humanized anti-CD39 antibody moieties and antigen-binding fragments thereof of the present disclosure comprise a light chain LFR1 comprising the sequence of X₈₆IVLTQSPATLX₈₇X₈₈SPGERX₈₉TX₉₀X₉₁C (SEQ ID NO: 150), or a homologous sequence of at least 80% sequence identity thereof; a light chain LFR2 comprising the sequence of WYQQKPGQX₁₀PX₁₁LLIY (SEQ ID NO: 81), or a homologous sequence of at least 80% sequence identity thereof; a light chain LFR3 comprises the sequence of GX₉₂PX₉₃RFSGSGSGTX₉₄X₉₅TLTISSX₉₆EPEDFAVYYC (SEQ ID NO: 148), or a homologous sequence of at least 80% sequence identity thereof, and a light chain LFR4 comprising the sequence of FGQGTKLEIK (SEQ ID NO: 83), or a homologous sequence of at least 80% sequence identity thereof, wherein X₁₀ and X₁₁ are as defined above, X₈₆ is E or Q; X₈₇ is S or T; X₈₈ is L or A; X₈₉ is A or V; X₉₀ is L or I; X₉₁ is S or T; X₉₂ is I or V; X₉₃ is A or T; X₉₄ is D or S; X₉₅ is F or Y; and X₉₆ is L or M.

In certain embodiments, the humanized anti-CD39 antibody moieties and antigen-binding fragments thereof of the present disclosure comprise a heavy chain HFR1 comprising a sequence selected from the group consisting of SEQ ID NOs: 84-86, 115, 119-120, and 131, a heavy chain HFR2 comprising the sequence of SEQ ID NOs: 87-90, and 121-123, a heavy chain HFR3 comprising a sequence selected from the group consisting of SEQ ID NOs: 91-97, 116-117, and 124-125, and a heavy chain HFR4 comprising a sequence selected from the group consisting of SEQ ID NOs: 79 and 118; and/or a light chain LFR1 comprising a sequence from the group consisting of SEQ ID NOs: 98-103 and 127-129, a light chain LFR2 comprising a sequence selected from the group consisting of SEQ ID NOs: 104, 105 and 130, a light chain LFR3 comprising a sequence selected from the group consisting of SEQ ID NOs: 106-110 and 132-133, and a light chain LFR4 comprising a sequence selected from the group consisting of SEQ ID NOs: 83 and 153.

In certain embodiments, the humanized anti-CD39 antibody moieties and antigen-binding fragments thereof of the present disclosure comprise HFR1, HFR2, HFR3, and/or HFR4 sequences contained in a heavy chain variable region selected from a group consisting of: hu14.VH_1 (SEQ ID NO: 68), hu14.VH_2 (SEQ ID NO: 70), hu14.VH_3 (SEQ ID NO: 72), hu14.VH_4 (SEQ ID NO: 74), hu23.VH_1 (SEQ ID NO: 60), hu23.VH_2 (SEQ ID NO: 62), hu23.VH_3 (SEQ ID NO: 64), hu23.VH_4 (SEQ ID NO: 66), hu23.VH_5 (SEQ ID NO: 140), hu23.VH_6 (SEQ ID NO: 141), hu23.VH_7 (SEQ ID NO: 142), hu23.201H (SEQ ID NO: 146), hu23.207H (SEQ ID NO: 147), and hu23.211H (SEQ ID NO: 39).

In certain embodiments, the humanized anti-CD39 antibody moieties and antigen-binding fragments thereof of the present disclosure comprise LFR1, LFR2, LFR3, and/or LFR4 sequences contained in a light chain variable region selected from a group consisting of: hu14.VL_1 (SEQ ID NO: 69), hu14.VL_2 (SEQ ID NO: 71), hu14.VL_3 (SEQ ID NO: 73), hu14.VL_4 (SEQ ID NO: 75), hu23.VL_1 (SEQ ID NO: 61), hu23.VL_2 (SEQ ID NO: 63), hu23.VL_3 (SEQ ID NO: 65), hu23.VL_4 (SEQ ID NO: 67), hu23.VL_5 (SEQ ID NO: 143), hu23.VL_6 (SEQ ID NO: 144), hu23.VL_7 (SEQ ID NO: 145), hu23.201L (SEQ ID NO: 111), hu23.203L (SEQ ID NO: 112), and hu23.211L (SEQ ID NO: 63).

In certain embodiments, the humanized anti-CD39 antibody moieties and antigen-binding fragments thereof provided herein comprise a heavy chain variable domain sequence selected from the group consisting of SEQ ID NOs: 39, 60, 62, 64, 66, 68, 70, 72, 74, 140, 141, 142, 146, 147; and/or a light chain variable domain sequence selected from the group consisting of SEQ ID NOs: 61, 63, 65, 67, 69, 71, 73, 75, 111, 112, 143, 144, and 145.

The exemplary humanized antibody moieties of chimeric antibody moiety c14 of the present disclosure include:
1) “hu14.H1L1” comprising the heavy chain variable region of hu14.VH_1 (SEQ ID NO: 68) and the light chain variable region of hu14.VL_1 (SEQ ID NO: 69);
2) “hu14.H2L1” comprising the heavy chain variable region of hu14.VH_2 (SEQ ID NO: 70) and the light chain variable region of hu14.VL_1 (SEQ ID NO: 69);
3) “hu14.H3L1” comprising the heavy chain variable region of hu14.VH_3 (SEQ ID NO: 72) and the light chain variable region of hu14.VL_1 (SEQ ID NO: 69);
4) “hu14.H4L1” comprising the heavy chain variable region of hu14.VH_4 (SEQ ID NO: 74) and the light chain variable region of hu14.VL_1 (SEQ ID NO: 69);
5) “hu14.H1L2” comprising the heavy chain variable region of hu14.VH_1 (SEQ ID NO: 68), and the light chain variable region of hu14.VL_2 (SEQ ID NO: 71);
6) “hu14.H2L2” comprising the heavy chain variable region of hu14.VH_2 (SEQ ID NO: 70), and the light chain variable region of hu14.VL_2 (SEQ ID NO: 71);
7) “hu14.H3L2” comprising the heavy chain variable region of hu14.VH_3 (SEQ ID NO: 72), and the light chain variable region of hu14.VL_2 (SEQ ID NO: 71);
8) “hu14.H4L2” comprising the heavy chain variable region of hu14.VH_4 (SEQ ID NO: 74), and the light chain variable region of hu14.VL_2 (SEQ ID NO: 71);
9) “hu14.H1L3” comprising the heavy chain variable region of hu14.VH_1 (SEQ ID NO: 68), and the light chain variable region of hu14.VL_3 (SEQ ID NO: 73);
“hu14.H2L3” comprising the heavy chain variable region of hu14.VH_2 (SEQ ID NO: 70), and the light chain variable region of hu14.VL_3 (SEQ ID NO: 73);
11) “hu14.H3L3” comprising the heavy chain variable region of hu14.VH_3 (SEQ ID NO: 72), and the light chain variable region of hu14.VL_3 (SEQ ID NO: 73);
12) “hu14.H4L3” comprising the heavy chain variable region of hu14.VH_4 (SEQ ID NO: 74), and the light chain variable region of hu14.VL_3 (SEQ ID NO: 73);
13) “hu14.H1L4” comprising the heavy chain variable region of hu14.VH_1 (SEQ ID NO: 68), and the light chain variable region of hu14.VL_4 (SEQ ID NO: 75);
14) “hu14.H2L4” comprising the heavy chain variable region of hu14.VH_2 (SEQ ID NO: 70), and the light chain variable region of hu14.VL_4 (SEQ ID NO: 75);
“hu14.H3L4” comprising the heavy chain variable region of hu14.VH_3 (SEQ ID NO: 72), and the light chain variable region of hu14.VL_4 (SEQ ID NO: 75);
16) “hu14.H4L4” comprising the heavy chain variable region of hu14.VH_4 (SEQ ID NO: 74), and the light chain variable region of hu14.VL_4 (SEQ ID NO: 75).

The exemplary humanized antibody moieties of chimeric antibody moiety c23 of the present disclosure include:
1) “hu23.H1L1” comprising the heavy chain variable region of hu23.VH_1 (SEQ ID NO: 60) and the light chain variable region of hu23.VL_1 (SEQ ID NO: 61);
2) “hu23.H2L1” comprising the heavy chain variable region of hu23.VH_2 (SEQ ID NO: 62) and the light chain variable region of hu23.VL_1 (SEQ ID NO: 61);
3) “hu23.H3L1” comprising the heavy chain variable region of hu23.VH_3 (SEQ ID NO: 64) and the light chain variable region of hu23.VL_1 (SEQ ID NO: 61);
4) “hu23.H4L1” comprising the heavy chain variable region of hu23.VH_4 (SEQ ID NO: 66) and the light chain variable region of hu23.VL_1 (SEQ ID NO: 61);
“hu23.H1L2” comprising the heavy chain variable region of hu23.VH_1 (SEQ ID NO: 60) and the light chain variable region of hu23.VL_2 (SEQ ID NO: 63);
6) “hu23.H2L2” comprising the heavy chain variable region of hu23.VH_2 (SEQ ID NO: 62) and the light chain variable region of hu23.VL_2 (SEQ ID NO: 63);
7) “hu23.H3L2” comprising the heavy chain variable region of hu23.VH_3 (SEQ ID NO: 64) and the light chain variable region of hu23.VL_2 (SEQ ID NO: 63);
8) “hu23.H4L2” comprising the heavy chain variable region of hu23.VH_4 (SEQ ID NO: 66) and the light chain variable region of hu23.VL_2 (SEQ ID NO: 63);
9) “hu23.H1L3” comprising the heavy chain variable region of hu23.VH_1 (SEQ ID NO: 60) and the light chain variable region of hu23.VL_3 (SEQ ID NO: 65);
“hu23.H2L3” comprising the heavy chain variable region of hu23.VH_2 (SEQ ID NO: 62) and the light chain variable region of hu23.VL_3 (SEQ ID NO: 65);
11) “hu23.H3L3” comprising the heavy chain variable region of hu23.VH_3 (SEQ ID NO: 64) and the light chain variable region of hu23.VL_3 (SEQ ID NO: 65);
12) “hu23.H4L3” comprising the heavy chain variable region of hu23.VH_4 (SEQ ID NO: 66) and the light chain variable region of hu23.VL_3 (SEQ ID NO: 65);
13) “hu23.H1L4” comprising the heavy chain variable region of hu23.VH_1 (SEQ ID NO: 60) and the light chain variable region of hu23.VL_4 (SEQ ID NO: 67);
14) “hu23.H2L4” comprising the heavy chain variable region of hu23.VH_2 (SEQ ID NO: 62) and the light chain variable region of hu23.VL_4 (SEQ ID NO: 67);
“hu23.H3L4” comprising the heavy chain variable region of hu23.VH_3 (SEQ ID NO: 64) and the light chain variable region of hu23.VL_4 (SEQ ID NO: 67);
16) “hu23.H4L4” comprising the heavy chain variable region of hu23.VH_4 (SEQ ID NO: 66) and the light chain variable region of hu23.VL_4 (SEQ ID NO: 67);
17) “hu23.H5L1” comprising the heavy chain variable region of hu23.VH_5 (SEQ ID NO: 140) and the light chain variable region of hu23.VL_1 (SEQ ID NO: 61);
18) “hu23.H6L1” comprising the heavy chain variable region of hu23.VH_6 (SEQ ID NO: 141) and the light chain variable region of hu23.VL_1 (SEQ ID NO: 61);
19) “hu23.H7L1” comprising the heavy chain variable region of hu23.VH_7 (SEQ ID NO: 142) and the light chain variable region of hu23.VL_1 (SEQ ID NO: 61);
“hu23.H1L5” comprising the heavy chain variable region of hu23.VH_1 (SEQ ID NO: 60) and the light chain variable region of hu23.VL_5 (SEQ ID NO: 143);
21) “hu23.H5L5” comprising the heavy chain variable region of hu23.VH_5 (SEQ ID NO: 140) and the light chain variable region of hu23.VL_5 (SEQ ID NO: 143);
22) “hu23.H6L5” comprising the heavy chain variable region of hu23.VH_6 (SEQ ID NO: 141) and the light chain variable region of hu23.VL_5 (SEQ ID NO: 143);
23) “hu23.H7L5” comprising the heavy chain variable region of hu23.VH_7 (SEQ ID NO: 142) and the light chain variable region of hu23.VL_5 (SEQ ID NO: 143);
24) “hu23.H1L6” comprising the heavy chain variable region of hu23.VH_1 (SEQ ID NO: 60) and the light chain variable region of hu23.VL_6 (SEQ ID NO: 144);
“hu23.H5L6” comprising the heavy chain variable region of hu23.VH_5 (SEQ ID NO: 140) and the light chain variable region of hu23.VL_6 (SEQ ID NO: 144);
26) “hu23.H6L6” comprising the heavy chain variable region of hu23.VH_6 (SEQ ID NO: 141) and the light chain variable region of hu23.VL_6 (SEQ ID NO: 144);
27) “hu23.H7L6” comprising the heavy chain variable region of hu23.VH_7 (SEQ ID NO: 142) and the light chain variable region of hu23.VL_6 (SEQ ID NO: 144);
28) “hu23.H1L7” comprising the heavy chain variable region of hu23.VH_1 (SEQ ID NO: 60) and the light chain variable region of hu23.VL_7 (SEQ ID NO: 145);
29) “hu23.H5L7” comprising the heavy chain variable region of hu23.VH_5 (SEQ ID NO: 140) and the light chain variable region of hu23.VL_7 (SEQ ID NO: 145);
“hu23.H6L7” comprising the heavy chain variable region of hu23.VH_6 (SEQ ID NO: 141) and the light chain variable region of hu23.VL_7 (SEQ ID NO: 145);
31) “hu23.H7L7” comprising the heavy chain variable region of hu23.VH_7 (SEQ ID NO: 142) and the light chain variable region of hu23.VL_7 (SEQ ID NO: 145);
32) “hu23.201” comprising the heavy chain variable region of hu23.201H (SEQ ID NO: 146) and the light chain variable region of hu23.201L (SEQ ID NO: 111);
33) “hu23.203” comprising the heavy chain variable region of hu23.201H (SEQ ID NO: 146) and the light chain variable region of hu23.203L (SEQ ID NO: 112);
34) “hu23.207” comprising the heavy chain variable region of hu23.207H (SEQ ID NO: 147) and the light chain variable region of hu23.201L (SEQ ID NO: 111);
“hu23.211” comprising the heavy chain variable region of hu23.211H (SEQ ID NO: 39) and the light chain variable region of hu23.211L (SEQ ID NO: 63).

These exemplary humanized anti-CD39 antibody moieties retained the specific binding capacity or affinity to CD39, and are at least comparable to, or even better than, the parent mouse antibody moiety mAb14 or mAb23 in that aspect.

In some embodiments, the anti-CD39 antibody moieties and antigen-binding fragments provided herein comprise all or a portion of the heavy chain variable domain and/or all or a portion of the light chain variable domain. In one embodiment, the anti-CD39 antibody moiety or an antigen-binding fragment thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the anti-CD39 antibody moieties or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-CD39 antibody moieties or the antigen-binding fragments thereof provided herein comprises heavy chain constant regions of human IgG1, IgG2, IgG3, IgG4, IgA1, IgA2 or IgM. In certain embodiments, the light chain constant region comprises Cκ or Cλ. The constant region of the anti-CD39 antibody moieties or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-CD39 antibody moieties and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region derived from human IgG1 with reduced effector functions. In some embodiments, the Fc region derived from human IgG1 comprises a L234A and/or L235A mutation. In certain embodiments, the anti-CD39 antibody moieties or antigen-binding fragments thereof provided herein comprise a wild type human IgG4 Fc region or other wild type human IgG4 alleles. In certain embodiments, the anti-CD39 antibody moieties or antigen-binding fragments thereof provided herein comprise a human IgG4 Fc region comprising a S228P mutation and/or a L235E mutation, and/or a F234A and L235A mutation. In some embodiments, the Fc region derived from human IgG4 comprises a S228P mutation and/or a F234A and L235A mutation.

In certain embodiments, the anti-CD39 antibody moieties or the antigen-binding fragments thereof provided herein have a specific binding affinity to human CD39 which is sufficient to provide for diagnostic and/or therapeutic use.

The anti-CD39 antibody moieties or antigen-binding fragments thereof provided herein can be a monoclonal antibody, a polyclonal antibody, a humanized antibody, a chimeric antibody, a recombinant antibody, a bispecific antibody, a multispecific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-CD39 antibody moiety or antigen-binding fragment thereof, which competes for binding to CD39 with the antibody moiety or antigen-binding fragment thereof provided herein. In certain embodiments, the present disclosure provides an anti-CD39 antibody moiety or antigen-binding fragment thereof, which competes for binding to human CD39 with an antibody moiety comprising a heavy chain variable region comprising the sequence of SEQ ID NO: 43, and a light chain variable region comprising the sequence of SEQ ID NO: 52. In certain embodiments, the present disclosure provides an anti-CD39 antibody moiety or antigen-binding fragment thereof, which competes for binding to human CD39 with an antibody moiety comprising a heavy chain variable region comprising the sequence of SEQ ID NO: 44, and a light chain variable region comprising the sequence of SEQ ID NO: 53. In certain embodiments, the present disclosure provides an anti-CD39 antibody moiety or antigen-binding fragment thereof, which competes for binding to human CD39 with an antibody moiety comprising a heavy chain variable region comprising the sequence of SEQ ID NO: 45, and a light chain variable region comprising the sequence of SEQ ID NO: 54, or competes for binding to human CD39 with an antibody moiety comprising a heavy chain variable region comprising the sequence of SEQ ID NO: 47, and a light chain variable region comprising the sequence of SEQ ID NO: 56.

In some embodiments, the present disclosure provides an anti-CD39 antibody moiety or an antigen-binding fragment thereof which specifically binds to an epitope of CD39, wherein the epitope comprises one or more residues selected from the group consisting of Q96, N99, E143, R147, R138, M139, E142, K5, E100, D107, V81, E82, R111, and V115.

In some embodiments, the epitope comprises one or more residues selected from the group consisting of Q96, N99, E143, and R147. In some embodiments, the epitope comprises all of the residues Q96, N99, E143, and R147.

In some embodiments, the epitope comprises one or more residues selected from the group consisting of R138, M139, and E142. In some embodiments, the epitope comprises all of the residues R138, M139, and E142.

In some embodiments, the epitope comprises one or more residues selected from the group consisting of K5, E100, and D107. In some embodiments, the epitope comprises all of the residues K5, E100, and D107.

In some embodiments, the epitope comprises one or more residues selected from the group consisting of V81, E82, R111, and V115. In some embodiments, the epitope comprises all of the residues V81, E82, R111, and V115.

In some embodiments, the CD39 is a human CD39. In some embodiments, the CD39 is a human CD39 comprising an amino acid sequence of SEQ ID NO: 162.

In certain embodiments, the anti-CD39 antibody moiety or antigen-binding fragment thereof provided herein is not any of Antibody 9-8B, Antibody T895, and Antibody I394.

“9-8B” as used herein refers to an antibody or antigen binding fragment thereof comprising a heavy chain variable region having an amino acid sequence of SEQ ID NO: 46, and a light chain variable region having an amino acid sequence of SEQ ID NO: 48.

“T895” as used herein refers to an antibody or antigen binding fragment thereof comprising a heavy chain variable region having an amino acid sequence of SEQ ID NO: 55, and a light chain variable region having an amino acid sequence of SEQ ID NO: 57.

“I394” as used herein refers to an antibody or antigen binding fragment thereof comprising a heavy chain variable region having an amino acid sequence of SEQ ID NO: 113, and a light chain variable region having an amino acid sequence of SEQ ID NO: 114.

### Antibody Variants

The anti-CD39 antibody moieties and antigen-binding fragments thereof provided herein also encompass various variants of the antibody sequences provided herein.

In certain embodiments, the antibody variants comprise one or more modifications or substitutions in one or more of the CDR sequences provided in Table 1 above, one or more of the non-CDR sequences of the heavy chain variable region or light chain variable region provided in Tables 4, 5, 8 and 9 above, and/or the constant region (e.g. Fc region). Such variants retain binding specificity to CD39 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variants

Affinity variants of antibodies may contain modifications or substitutions in one or more CDR sequences provided in Table 1 above, one or more FR sequences provided in Tables 4, 5, 8, and 9 above, or the heavy or light chain variable region sequences provided in Tables 2, 3, 6 and 7 above. FR sequences can be readily identified by a person skilled in the art based on the CDR sequences in Table 1 above and variable region sequences in Tables 2, 3, 6 and 7 above, as it is well-known in the art that a CDR region is flanked by two FR regions in the variable region. The affinity variants retain specific binding affinity to CD39 of the parent antibody, or even have improved CD39 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 1 above, and variable region sequences provided in Tables 2, 3, 6 and 7 above, one or more amino acid residues may be substituted yet the resulting antibody or antigen-binding fragment still retain the binding affinity or binding capacity to CD39, or even have an improved binding affinity or capacity. Various methods known in the art can be used to achieve this purpose. For example, a library of antibody variants (such as Fab or scFv variants) can be generated and expressed with phage display technology, and then screened for the binding affinity to human CD39. For another example, computer software can be used to virtually simulate the binding of the antibodies to human CD39, and identify the amino acid residues on the antibodies which form the binding interface. Such residues may be either avoided in the substitution so as to prevent reduction in binding affinity, or targeted for substitution to provide for a stronger binding.

In certain embodiments, the humanized anti-CD39 antibody moiety or antigen-binding fragment thereof provided herein comprises one or more amino acid residue substitutions in one or more of the CDR sequences, and/or one or more of the FR sequences. In certain embodiments, an affinity variant comprises no more than 15, 10, 9, 8, 7, 6, 5, 4, 3, 2, or 1 substitutions in the CDR sequences and/or FR sequences in total.

In certain embodiments, the anti-CD39 antibody moieties or antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1 above yet retaining the specific binding affinity to CD39 at a level similar to or even higher than its parent antibody.

In certain embodiments, the anti-CD39 antibody moieties or antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Tables 2, 3, 6 and 7 above yet retaining the specific binding affinity to CD39 at a level similar to or even higher than its parent antibody. In some embodiments, a total of 1 to 10 amino acids have been substituted, inserted, or deleted in a variable region sequence listed in Tables 2, 3, 6 and 7 above. In some embodiments, the substitutions, insertions, or deletions occur in regions outside the CDRs (e.g. in the FRs).

### Glycosylation Variants

The anti-CD39 antibody moieties or antigen-binding fragments thereof provided herein also encompass glycosylation variants, which can be obtained to either increase or decrease the extent of glycosylation of the antibodies or antigen binding fragments thereof.

The anti-CD39 antibody moieties or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-CD39 antibody moieties and antigen-binding fragments provided herein comprise one or more mutations at a position selected from the group consisting of N55, G56, and N297, to remove one or more deamidation site. In certain embodiments, the anti-CD39 antibody moieties and antigen-binding fragments provided herein comprise a mutation at N55 (for example, N55G, N55S or N55Q), and/or a mutation at G56 (for example, G56A, G56D), and/or a mutation at N297 (for example, N297A, N297Q, or N297G). These mutations are tested and are believed not to negatively affect the binding affinity of the antibody moieties provided herein.

### Cysteine-Engineered Variants

The anti-CD39 antibody moieties or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-CD39 antibody moieties or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibody moieties or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fe region variants. One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-CD39 antibody moieties or antigen-binding fragments thereof provided herein has reduced effector functions, and comprise one or more amino acid substitution(s) in IgG1 at a position selected from the group consisting of: 234, 235, 237, 238, 268, 297, 309, 330, and 331. In certain embodiments, the anti-CD39 antibody moieties or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof. In certain embodiments, the anti-CD39 antibody moieties or antigen-binding fragments thereof provided herein is of IgG1 isotype and comprise a L234A and L235A mutation. In certain embodiments, the anti-CD39 antibody moieties or antigen-binding fragments thereof provided herein is of IgG2 isotype, and comprises one or more amino acid substitution(s) selected from the group consisting of: H268Q, V309L, A330S, P331S, V234A, G237A, P238S, H268A, and any combination thereof (e.g. H268Q/V309L/A330S/P331S, V234A/G237A/P238S/H268A/V309L/A330S/P331S). In certain embodiments, the anti-CD39 antibody moieties or antigen-binding fragments thereof provided herein is of IgG4 isotype, and comprises one or more amino acid substitution(s) selected from the group consisting of: S228P, N297A, N297Q, N297G, L235E, F234A, L235A, and any combination thereof. In certain embodiments, the anti-CD39 antibody moieties or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the anti-CD39 antibody moieties and antigen-binding fragments thereof provided herein is of IgG4 isotype and comprises one or more amino acid substitution(s) at one or more points of 228, 234 and 235. In certain embodiments, the anti-CD39 antibody moieties and antigen-binding fragments provided herein is of IgG4 isotype and comprises a S228P mutation and/or a L235E mutation and/or a F234A and L235A mutation in the Fc region.

In certain embodiments, the anti-CD39 antibody moieties or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, anti-CD39 antibody moieties or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

### Antigen-Binding Fragments

Provided herein are also anti-CD39 antigen-binding fragments. Various types of antigen-binding fragments are known in the art and can be developed based on the anti-CD39 antibody moieties provided herein, including for example, the exemplary antibody moieties whose CDRs are shown in Table 1 above, and variable sequences are shown in Tables 2, 3, 6 and 7, and their different variants (such as affinity variants, glycosylation variants, Fc variants, cysteine-engineered variants and so on).

In certain embodiments, an anti-CD39 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. coli (e.g. for Fab, Fv and ScFv antibody fragments), screening from a phage display library as discussed above (e.g. for ScFv), and chemical coupling of two Fab′-SH fragments to form F(ab′) 2 fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-CD39 antibody moieties or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-CD39 antibody moieties or antigen-binding fragments thereof is bispecific. In certain embodiments, the anti-CD39 antibody moieties or antigen-binding fragment thereof is further linked to a second functional moiety having a different binding specificity from said anti-CD39 antibody moiety, or antigen binding fragment thereof.

In certain embodiments, the bispecific antibodies or antigen-binding fragments thereof provided herein are capable of specifically binding to a second antigen other than CD39, or a second epitope on CD39. In certain embodiments, the second antigen is selected from the group consisting of TGFbeta, CD73, PD1, PDL1, 4-1BB, CTLA4, TIGIT, GITA, VISTA, TIGIT, B7-H3, B7-H4, B7-H5, CD112R, Siglec-15, LAG3, SIRPα, CD47 and TIM-3.

### Conjugates

In some embodiments, the anti-CD39 antibody moieties or antigen-binding fragments thereof further comprise one or more conjugate moieties. The conjugate moiety can be linked to the antibody moieties or antigen-binding fragments thereof. A conjugate moiety is a moiety that can be attached to the antibody moiety or antigen-binding fragment thereof. It is contemplated that a variety of conjugate moieties may be linked to the antibodies moiety or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibody moieties or antigen-binding fragments thereof by covalent binding, affinity binding, intercalation, coordinate binding, complexation, association, blending, or addition, among other methods. In some embodiments, the anti-CD39 antibody moieties or antigen-binding fragments thereof can be linked to one or more conjugates via a linker.

In certain embodiments, the anti-CD39 antibody moieties or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the anti-CD39 antibody moieties or antigen-binding fragments thereof may be linked to a conjugate moiety indirectly, or through another conjugate moiety. For example, the anti-CD39 antibody moieties or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other anticancer drugs.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. 123I, 124I, 125I, 131I, 35S, 3H, 111In, 112In, 14C, 64Cu, 67Cu, 86Y, 88Y, 90Y, 177Lu, 211At, 186Re, 188Re, 153Sm, 212Bi, and 32P, other lanthanides), luminescent labels, chromophoric moieties, digoxigenin, biotin/avidin, DNA molecules or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the anti-CD39 antibody moieties or antigen-binding fragments thereof provided herein is used as a base for a conjugate.

### Polynucleotides and Recombinant Methods

The present disclosure provides isolated polynucleotides that encode the anti-CD39/TGFβ Trap provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-CD39/TGFβ Trap provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors comprising the isolated polynucleotides provided herein. In certain embodiments, the polynucleotide provided herein encodes the anti-CD39/TGFβ Trap provided herein, at least one promoter (e.g. SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the anti-CD39/TGFβ Trap provided herein can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for vectors encoding the anti-CD39/TGFβ Trap. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K bulgaricus (ATCC 16,045), K. wickeramii (ATCC 24,178), K. waltii (ATCC 56,500), K. drosophilarum (ATCC 36,906), K. thermotolerans, and K. marxianus; Yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-CD39/TGFβ Trap production and cultured in conventional nutrient media modified as appropriate for inducing promoters, selecting transformants, or amplifying the genes encoding the desired sequences. In another embodiment, the anti-CD39/TGFβ Trap may be produced by homologous recombination known in the art. In certain embodiments, the host cell is capable of producing the anti-CD39/TGFβ Trap provided herein.

The present disclosure also provides a method of expressing the anti-CD39/TGFβ Trap provided herein, comprising culturing the host cell provided herein under the condition at which the vector of the present disclosure is expressed. The host cells used to produce the anti-CD39/TGFβ Trap provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the anti-CD39/TGFβ Trap can be produced intracellularly, in the periplasmic space, or directly secreted into the medium. If the anti-CD39/TGFβ Trap is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD39/TGFβ Trap prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the anti-CD39/TGFβ Trap. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

### Pharmaceutical Composition

The present disclosure further provides pharmaceutical compositions comprising the anti-CD39/TGFβ Trap and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising the anti-CD39/TGFβ Trap and conjugates provided herein decreases oxidation of the anti-CD39/TGFβ Trap. This reduction in oxidation prevents or reduces loss of binding affinity, thereby improving antibody stability and maximizing shelf-life. Therefore, in certain embodiments, pharmaceutical compositions are provided that comprise one or more anti-CD39/TGFβ Traps as disclosed herein and one or more antioxidants such as methionine. Further provided are methods for preventing oxidation of, extending the shelf-life of, and/or improving the efficacy of the anti-CD39/TGFβ Trap provided herein by mixing the anti-CD39/TGFβ Trap with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-CD39/TGFβ Trap or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the anti-CD39/TGFβ Trap provided herein and/or the pharmaceutical composition provided herein. In certain embodiments, the present disclosure provides a kit comprising the anti-CD39/TGFβ Trap provided herein, and a second therapeutic agent. In certain embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a CD39 related and/or a TGFβ related disease, disorder or condition in a subject, comprising administering to the subject a therapeutically effective amount of the anti-CD39/TGFβ Trap provided herein, and/or the pharmaceutical composition provided herein. In certain embodiments, the subject is human. The present inventors unexpectedly found that synergic effect can be achieved in treating, preventing or alleviating a CD39 related and/or a TGFβ related disease, disorder or condition in a subject by simultaneously blocking adenosine pathway (through the inhibition of CD39) and blocking TGFβ signaling pathway (via TGFβ trap).

In some embodiments, the CD39 related disease, disorder or condition is characterized in expressing or over-expressing of CD39. In some embodiments, the TGFβ related disease, disorder or condition is characterized in expressing or over-expressing of TGFβ.

In certain embodiments, the CD39 related disease, disorder or condition is cancer. In certain embodiments, the cancer is a CD39-expressing cancer. “CD39-expressing” cancer as used herein refers to a cancer characterized in expressing CD39 protein in a cancer cell, a tumor infiltrating immune cell or an immune suppression cell, or expressing CD39 in a cancer cell, a tumor infiltrating immune cell or an immune suppression cell at a level significantly higher than that would have been expected of a normal cell. Various methods can be used to determine the presence and/or amount of CD39 in a test biological sample from the subject. For example, the test biological sample can be exposed to anti-CD39 antibody or antigen-binding fragment thereof, which binds to and detects the expressed CD39 protein. Alternatively, CD39 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In certain embodiments, the TGFβ related disease, disorder or condition is cancer. In certain embodiments, the cancer is a TGFβ-expressing cancer. “TGFβ-expressing” cancer as used herein refers to a cancer characterized in expressing TGFβ protein in a cancer cell, a tumor infiltrating immune cell or an immune suppression cell, or expressing TGFβ in a cancer cell, a tumor infiltrating immune cell or an immune suppression cell at a level significantly higher than that would have been expected of a normal cell.

The present disclosure also provides methods of treating, preventing or alleviating a disease associated with an increased level and/or activity of TGFβ in a subject, comprising administering to the subject a therapeutically effective amount of the anti-CD39/TGFβ Trap provided herein and/or the pharmaceutical composition provided herein.

Various methods can be used to determine the presence and/or amount of TGFβ in a test biological sample from the subject. For example, the test biological sample can be exposed to anti-TGFβ antibody or antigen-binding fragment thereof, which binds to and detects the expressed TGFβ protein. Alternatively, TGFβ can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In certain embodiments, the disease, disorder or condition above is cancer, pancreatic atrophy, or fibrosis.

In certain embodiments, the cancer is selected from the group consisting of anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma. In certain embodiments, the cancer is a leukemia, lymphoma, bladder cancer, glioma, glioblastoma, ovarian cancer, melanoma, prostate cancer, thyroid cancer, esophageal cancer or breast cancer.

TGFβ is the primary factor that drives fibrosis in most, if not all, forms of chronic kidney disease (CKD). Inhibition of the TGF-β isoform, TGF-β1, or its downstream signaling pathways substantially limits renal fibrosis in a wide range of disease models whereas overexpression of TGF-β1 induces renal fibrosis. TGF-β1 can induce fibrosis via activation of both canonical (Smad-based) and non-canonical (non-Smad-based) signaling pathways, which result in activation of myofibroblasts, excessive production of extracellular matrix (ECM) and inhibition of ECM degradation. The role of Smad proteins in the regulation of fibrosis is complex, with competing profibrotic and antifibrotic actions (including in the regulation of mesenchymal transitioning), and with complex interplay between TGF-β/Smads and other signalling pathways. Studies have identified additional mechanisms that regulate the action of TGF-β1/Smad signalling in fibrosis, including short and long noncoding RNA molecules and epigenetic modifications of DNA and histone proteins. Although direct targeting of TGF-β1 is unlikely to yield a viable antifibrotic therapy due to the involvement of TGF-β1 in other processes, greater understanding of the various pathways by which TGF-β1 controls fibrosis has identified alternative targets for the development of novel therapeutics to halt this most damaging process in CKD.

Adenosine has an important role in inflammation and tissue remodeling and promotes dermal fibrosis by adenosine receptor (A2AR) activation. Extracellular adenosine, generated in tandem by ecto-enzymes CD39 and CD73, promotes dermal fibrogenesis. The adenosine axis is involved in renal ischemia reperfusion injury (IRI) and the generation of adenosine by the action of CD39 and CD73 is protective. However, chronic elevation of adenosine has been linked to the development of renal fibrosis. The evidence showed that deletion of CD39 and/or CD73 decreased the collagen content, and prevented skin thickening and tensile strength increase after bleomycin challenge. Decreased dermal fibrotic features were associated with reduced expression of the profibrotic mediators, transforming growth factor-β1 and connective tissue growth factor, and diminished myofibroblast population in CD39- and/or CD73-deficient mice.

We hypothesize that inhibition of CD39 and TGF-β may hold promise in the treatment of fibrosis in diseases such as scleroderma, liver and renal fibrosis.

In certain embodiments, the fibrosis is selected from the group consisting of scleroderma, renal fibrosis, pulmonary fibrosis (e.g. cystic fibrosis, idiopathic pulmonary fibrosis), liver fibrosis (e.g. bridging fibrosis, cirrhosis), brain fibrosis, arthrofibrosis, mediastinal fibrosis, myelofibrosis, nephrogenic systemic fibrosis, retroperitoneal fibrosis, and myocardial fibrosis (e.g. interstitial fibrosis, replacement fibrosis). In some embodiments, the subject has been identified as having a cancer cell or tumor infiltrating immune cells or immune suppression cells expressing CD39 and/or TGFβ, optionally at a level significantly higher from the level normally found on non-cancer cells or non-immune suppression cells.

In some embodiments, the immune suppression cells are regulatory T cells. Regulatory T cells (“Tregs”) are a distinct population of T lymphocytes that have the capacity to dominantly suppress the proliferation of responder T cells in vitro and inhibit autoimmune disease in vivo. Tregs of the present disclosure can be CD4+CD25⁺ FoxP3⁺ T cells with suppressive properties. In certain embodiments, the Tregs of the present disclosure are CD4⁺ Tregs, in particular, CD4⁺ Tregs overexpressing CD39.

In some embodiments, the subject has been identified as having an overactive regulatory T cell in tumor microenvironment compared to the activity of a regulatory T cell normally found in a control subject. The activity of regulatory T cell in tumor microenvironment can be determined by conventional methods in the art, for example, up-regulation of CD25⁺Foxp3⁺ on T cells, secretion of TGFβ and IL-10, inhibition of CTL cytotoxicity, etc.

In some embodiments, the subject is expected to be beneficial from the reversion of immunosuppression, or the reversion of dysfunctional exhausted T cells.

In some embodiments, the disease, disorder or condition is an autoimmune disease or infection. In some embodiments, the autoimmune disease is immune thrombocytopenia, systemic scleroderma, sclerosis, adult respiratory distress syndrome, eczema, asthma, Sjogren's syndrome, Addison's disease, giant cell arteritis, immune complex nephritis, immune thrombocytopenic purpura, autoimmune thrombocytopenia, Celiac disease, psoriasis, dermatitis, colitis or systemic lupus erythematosus. In some embodiments, the infection is a viral infection or a bacterial infection. In some embodiments, the infection is HIV infection, HBV infection, HCV infection, inflammatory bowel disease, or Crohn's disease.

In another aspect, methods are provided to treat, prevent or alleviate a disease, disorder or condition in a subject that would benefit from modulation of CD39 activity and/or TGFβ activity, comprising administering a therapeutically effective amount of the anti-CD39/TGFβ Trap provided herein and/or the pharmaceutical composition provided herein to a subject in need thereof. In certain embodiments, the disease, disorder or condition is a CD39 related and/or TGFβ related disease, disorder or condition, which is defined above.

The therapeutically effective amount of an anti-CD39/TGFβ Trap provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the anti-CD39/TGFβ Trap provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The anti-CD39/TGFβ Trap provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the anti-CD39/TGFβ Trap provided herein may be administered alone or in combination with a therapeutically effective amount of a second therapeutic agent. For example, the anti-CD39/TGFβ Trap disclosed herein may be administered in combination with a second therapeutic agent, for example, a chemotherapeutic agent, an anti-cancer drug, radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, or cytokines.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, the anti-CD39/TGFβ Trap provided herein that is administered in combination with one or more additional therapeutic agents may be administered simultaneously with the one or more additional therapeutic agents, and in certain of these embodiments the anti-CD39/TGFβ Trap and the additional therapeutic agent(s) may be administered as part of the same pharmaceutical composition. However, an anti-CD39/TGFβ Trap administered “in combination” with another therapeutic agent does not have to be administered simultaneously with or in the same composition as the agent. An anti-CD39/TGFβ Trap administered prior to or after another agent is considered to be administered “in combination” with that agent as the phrase is used herein, even if the anti-CD39/TGFβ Trap and the second agent are administered via different routes. Where possible, additional therapeutic agent(s) administered in combination with the anti-CD39/TGFβ Trap disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of modulating CD39 activity in CD39-positive cells, comprising exposing the CD39-positive cells to the anti-CD39/TGFβ Trap provided herein. In some embodiments, the CD39-positive cell is an immune cell.

The present disclosure further provides methods for modulating TGFβ activity in TGFβ-positive cells, comprising exposing the TGFβ-positive cells to the anti-CD39/TGFβ Trap provided herein.

In another aspect, the present disclosure provides methods of detecting the presence or amount of CD39 and/or TGFβ in a sample, comprising contacting the sample with the anti-CD39/TGFβ Trap provided herein and/or the pharmaceutical composition provided herein, and determining the presence or the amount of CD39 and/or TGFβ in the sample.

In another aspect, the present disclosure provides a method of diagnosing a CD39 related and/or a TGFβ related disease, disorder or condition in a subject, comprising: a) contacting a sample obtained from the subject with the anti-CD39/TGFβ Trap provided herein and/or the pharmaceutical composition provided herein; b) determining the presence or amount of CD39 and/or TGFβ in the sample; and c) correlating the presence or the amount of CD39 and/or TGFβ to existence or status of the CD39 related and/or a TGFβ related disease, disorder or condition in the subject.

In another aspect, the present disclosure provides kits comprising the anti-CD39/TGFβ Trap provided herein and/or the pharmaceutical composition provided herein, optionally conjugated with a detectable moiety, which is useful in detecting a CD39 related and/or a TGFβ related disease, disorder or condition. The kits may further comprise instructions for use.

In another aspect, the present disclosure also provides use of the anti-CD39/TGFβ Trap provided herein and/or the pharmaceutical composition provided herein in the manufacture of a medicament for treating, preventing or alleviating a CD39 related and/or a TGFβ related disease, disorder or condition in a subject, in the manufacture of a diagnostic reagent for diagnosing a CD39 related and/or a TGFβ related disease, disorder or condition.

In another aspect, the present disclosure provides a method of treating, preventing or alleviating a disease treatable by reducing the ATPase activity of CD39 in a subject, comprising administering to the subject a therapeutically effective amount of the anti-CD39/TGFβ Trap provided herein and/or the pharmaceutical composition provided herein. For example, the anti-CD39/TGFβ Trap provided herein may be administered to reduce the ATPase activity of cancer cells, tumor infiltrating immune cells, immune suppression cells that express CD39. In some embodiments, the subject is human. In some embodiments, the subject has a disease, disorder or condition selected from the group consisting of cancer, pancreatic atrophy, fibrosis, an autoimmune disease, and an infection.

In another aspect, the present disclosure provides a method of treating, preventing or alleviating a disease associated with adenosine-mediated inhibition of T cell, Monocyte, Macrophage, DC, APC, NK and/or B cell activity in a subject, comprising administering to the subject a therapeutically effective amount of the anti-CD39/TGFβ Trap provided herein and/or the pharmaceutical composition provided herein.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Materials Generation

1.1. Reference Antibody Generation

Anti-CD39 reference antibodies were generated based on the published sequences. Antibody 9-8B was disclosed in patent application WO 2016/073845A1, and its heavy and light chain variable region sequences are included herein as SEQ ID NOs: 46 and 48, respectively. Antibody T895 was disclosed as antibody 31895 in patent application WO 2019/027935A1, and its heavy and light chain variable region sequences are included herein as SEQ ID NOs: 55 and 57, respectively. Antibody I394 was disclosed in the patent application WO 2018/167267A1, and its heavy and light chain variable region sequences are included herein as SEQ ID NOs: 113 and 114, respectively. The heavy chain and light chain variable regions of Antibodies 9-8B, T895, and I394 are shown in Table 10 below. The DNA sequences encoding the reference antibodies were cloned and expressed in Expi293 cells (Invitrogen). The cell culture medium was collected and centrifuged to remove cell pellets. The harvested supernatant was purified using Protein A affinity chromatography column (Mabselect Sure, GE Healthcare) to obtain the reference antibody preparations.

TABLE 10
Variable region amino acid sequences of 3 reference antibodies.

| Antibody | VH | VL |
|---|---|---|
| 9-8B | SEQ ID NO: 46 | SEQ ID NO: 48 |
| | QIQLVQSGPELKKPGETVKISC | DIVMTQSQKFMSTSVGDRVSV |
| | KASGYTFTHYGMNWVKQAPG | TCKASHNVGTNVAWYQQKPG |
| | KGLKWMGWINTYTGELTYAD | QSPKALIYSASYRYSGVPGRFT |
| | DFKGRFAFSLETSASTAYLQIN | GSGSGTDFTLTISNVQSEDLAE |
| | NLKNEDTATYFCARRAYYRYD | YFCHQYNNYPYTFGGGTKLEI |
| | YVMDYWGQGTSVTVSS | K |
| T895 | SEQ ID NO: 55 | SEQ ID NO: 57 |
| | EVQLQQSGPELVKPGASVKMS | DIVLTQSPASLAVSLGQRATIS |
| | CKASGYTFTDYNMHWVKQSH | CRASESVDNFGVSFMYWFQQ |
| | GRTLEWIGYIVPLNGGSTFNQK | KPGQPPNLLIYGASNQGSGVP |
| | FKGRATLTVNTSSRTAYMELRS | ARFRGSGSGTDFSLNIHPMEA |
| | LTSEDSAAYYCARGGTRFAYW | DDTAMYFCQQTKEVPYTFGG |
| | GQGTLVTVSA | GTKLEIK |
| I394 | SEQ ID NO: 113 | SEQ ID NO: 114 |
| | QVQLVQSGAEVKKPGASVKVS | EIVLTQSPGTLSLSPGERATLSC |
| | CKASGYTFKSYEMHWVRQAP | RASQSVASSYLAWYQQKPGQ |
| | GQGLEWMGRINPSVGSTWYA | APRLLIYGASNRHTGIPDRESG |
| | QKFQGRVTMTRDTSTSTVYME | SGSGTDFTLTISRLEPEDFAVY |
| | LSSLRSEDTAVYYCARGKREG | YCQQYHNAITFGGGTKVEIK |
| | GTEYLRKWGQGTLVTVSS | |

1.2. Generation of Human, Cynomolgus Monkey, and Mouse CD39 Stable Expression Cell Lines

The DNA sequences encoding full length human CD39 (NP_001767.3), cyno CD39 (XP_015311944.1) and mouse CD39 (NP_033978.1) respectively were cloned into an expression vector, followed by transfection and expression in HEK293 cells. The transfected cells expressing human CD39, cyno CD39 and mouse CD39 respectively were cultured in a selective medium. Single cell clones stably expressing human CD39, cyno CD39 or mouse CD39 were isolated by limiting dilution. The cells were subsequently screened by FACS using anti-human CD39 antibody (BD, Cat #555464), anti-cyno CD39 (9-8B), anti-mouse CD39 (Biolegend, Cat #143810).

In a similar way, CHOK1 cells (Invitrogen) transfected with human CD39, cyno CD39 or mouse CD39 expression plasmid were cultured in a selective medium. Single cell clones stably expressing human CD39, cyno CD39 or mouse CD39 were isolated by limiting dilution, and subsequently screened by FACS using the anti-human CD39 antibody, the anti-cyno CD39 antibody or the anti-mouse CD39 antibody.

The stable cell lines were designated as HEK293-hCD39, HEK293-cynoCD39, HEK293-mCD39, CHOK1-hCD39, CHOK1-cynoCD39, and CHOK1-mCD39, respectively, all of which showed high expression and ATPase activity.

1.3. Recombinant Proteins Generation

The DNA sequence encoding extracellular domain (ECD) of human CD39 was cloned into the expression vector, and was transfected into HEK293 cells to allow expression of the recombinant ECD protein.

### Example 2. Antibody Generation

2.1 Immunization and Hybridoma Generation and Screening

To generate antibodies to CD39, Balb/c and SJL/J mice (SLAC) were immunized with recombinantly expressed human CD39 antigen or its fragments, or DNA encoding full length human CD39 and/or cells expressing human CD39. The immune response was monitored over the course of the immunization protocol with plasma and serum samples were obtained by tail vein or retroorbital bleeds. Mice with sufficient titers of anti-CD39 antibodies were used for fusions. Splenocytes and/or lymph node cells from immunized mice were isolated and fused to mouse myeloma cell line (SP2/0). The resulting hybridomas were screened for the production of CD39-specific antibodies, by ELISA assay with human CD39 ECD recombinant protein, or by Acumen assay (TTP Labtech) with CHOK1-hCD39 cells stably expressing human CD39. Hybridoma clones specific to hCD39 were confirmed by FACS and enzyme activity blocking assay, and were subcloned to get stable hybridoma clones. After 1-2 rounds of subcloning, hybridoma monoclones were expanded for antibody production and frozen as stock.

The antibody secreting hybridomas were subcloned by limiting dilution. The stable subclones were cultured in vitro to generate antibody in tissue culture medium for characterization. After 1-2 rounds of subcloning, hybridoma monoclones were expanded for antibody production.

After about 14 days of culturing, the hybridoma cell culture medium were collected and purified by Protein A affinity chromatography column (GE). The hybridoma antibody clones were designated as mAb13, mAb14, mAb19, mAb21, mAb23, mAb34 and mAb35, respectively.

### Example 3. Antibody Characterization

3.1. Antibodies

The hybridoma antibody clones mAb13, mAb14, mAb19, mAb21, mAb23, mAb34 and mAb35 were characterized in a series of binding and functional assays as described below.

3.2. Binding Affinity to Human CD39, Cynomolgus CD39 and Mouse CD39

FACS were used to determine binding of the antibodies to cell lines expressing CD39 naturally (SK-MEL-28) or recombinantly (CHOK1-hCD39, CHOK1-cynoCD39, and CHOK1-mCD39), or with cells lacking CD39 expression (CHOK1-blank) as a negative control.

CHOK1-hCD39, CHOK1-cCD39, CHOK1-mCD39 and CHOK1-blank cells were maintained in culture medium according to ATCC procedure. Cells were collected and re-suspended in blocking buffer at a density of 3×10⁶ cells/ml. Cells were transferred to 96 well FACS plates at 100 μl/well (3×10⁵ cells/well), the plates were centrifuged and washed twice with FACS buffer (PBS, 1% FBS, 0.05% Tween-4-folds serial dilution of anti-CD39 antibodies were prepared in FACS buffer starting from 30 μg/ml. Reference antibody 9-8B and mouse/human control IgG were used as positive and negative controls, respectively. Cells were re-suspended in 100 μL/well diluted antibodies, and the plates were incubated at 4° C. for 60 min. The plates were washed with FACS buffer, Alexa Fluor® 488-labeled secondary antibody (1:1000 in FACS buffer) were added to each well and incubated at 4° C. for 30 min. The plates were washed with FACS buffer, and cells were re-suspended in 100 μL/well of PBS. Cells were then analyzed with FACSVerse™ and mean fluorescence intensity were determined. Full binding curves were generated on the CD39 expressing cells by testing a range of antibody concentrations. Apparent affinity was determined for each antibody using Prism software.

Similarly, human CD39 expressing cells SK-MEL-5, SK-MEL-28 or MOLP-8, were incubated with a gradient concentration of anti-CD39 antibodies for 30 minutes at 4° C. Cells were washed 3 times using FACS buffer and next incubated with fluorescence labelled secondary antibody (goat-anti-mouse IgG or goat anti-human IgG) for 30 minutes at 4° C. Cells were washed 3 times and then re-suspended in FACS buffer and analyzed by flow cytometry analysis on BD Celesta. Data plotted and analyzed using GraphPad Prism 8.02.

The binding affinity of the 7 purified hybridoma antibodies is summarized in Table 11, in comparison with known anti-CD39 antibody 9-8B. All the hybridoma antibodies bound to human and cynomolgus CD39 in a dose-dependent manner, however none recognized mouse CD39 in the FACS study.

TABLE 11
Anti-CD39 bybridoma antibodies characterization summary.

| | | | | | SK-MEL-28 cell | | | | | | |
|---|---|---|---|---|---|---|---|---|---|---|---|
| | Binding affinity EC₅₀ (M) | | | | based ATPase | | T Cell Proliferation | | Octet Binding | | |
| ES- | | | | SK- | inhibition % | | Suppression Assay | | Affinity | | |
| Number | human | cynomolgus | mouse | MEL-28 | 10 nM | EC₅₀ | 100 nM | EC₅₀ | KD | K-off | epitope |
| mAb13 | 3.0E−08 | 2.1E−08 | — | 1.1E−08 | 43% | 3.5E−10 | n.d. | n.d. | n.d. | n.d. | n.d. |
| mAb14 | 2.5E−09 | 3.4E−09 | — | 2.8E−08 | 57% | 4.5E−09 | ++ | n.d. | 2.4E−10 | 2.2E−04 | IV |
| mAb19 | 4.5E−09 | 5.8E−09 | — | 4.6E−08 | 65% | 4.0E−09 | ++ | n.d. | 2.6E−10 | 1.5E−04 | I |
| mAb21 | 6.7E−09 | 1.1E−08 | — | 9.0E−08 | 70% | 1.5E−09 | ++ | 1.4E−10 | 4.4E−10 | 2.2E−04 | I |
| mAb23 | 1.7E−09 | 2.3E−09 | — | 1.6E−09 | 75% | 6.0E−11 | ++ | ~1.0E−10 | 8.2E−10 | 3.6E−04 | II |
| mAb34 | 4.4E−09 | 3.1E−09 | — | 4.7E−09 | 72% | 1.3E−10 | n.d. | n.d. | n.d. | n.d. | n.d. |
| mAb35 | 5.2E−09 | 5.3E−09 | — | 1.8E−09 | 56% | 1.6E−09 | n.d. | n.d. | n.d. | n.d. | n.d. |
| 9-8B | 1.7E−09 | 1.6E−09 | — | 8.0E−10 | 21% | 5.0E−11 | n.d. | n.d. | n.d. | n.d. | III |

−: negative
++: p < 0.01
n.d.: not determined yet

3.3. ATPase Inhibition Detection

CD39 expressing cells, SK-MEL-5 and MOLP-8 were washed with PBS buffer and incubated with a gradient of antibodies for 30 minutes at 37° C. 50 mM ATP was added to each well and incubated with cells for 16 hours. The supernatants were collected and the orthophosphate product from ATP degradation was measured by a Malachite Green Phosphate Detection Kit (R&D systems, Catalog #DY996) according to manufacturer's manual. Isotype and/or 9-8B was used as control. Data plotted and analyzed using GraphPad Prism 8.02. EC₅₀ is the concentration of the indicated antibody to reach 50% of the signal in this assay.

As summarized in Table 11, all 7 purified hybridoma antibodies had good ATPase inhibition activity compared with reference antibody 9-8B.

3.4. ATP-Mediated T Cell Proliferation Suppression Assay

Human T cells labeled with CSFE and stimulated with anti-CD3 and anti-CD28 were incubated with anti-CD39 antibodies or isotype control in the presence of ATP. Proliferation of T cells was analyzed in FACS by CSFE dilution. mIgG2a was used as an isotype control.

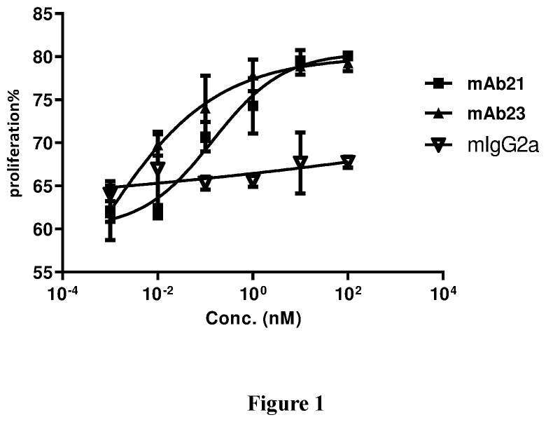

The T cell proliferation activity of selected anti-CD39 antibodies mAb21 and mAb23 were shown in FIG. 1 and summarized in Table 11. EC₅₀ is the concentration of the indicated antibody to reach 50% of the signal in this assay. Both antibodies enhanced the T cell proliferation in a dose-dependent manner, that is, both antibodies blocked the ATP-mediated inhibition on T cell proliferation.

3.5. Epitope Binning

Anti-CD39 antibodies were labeled using Alex488 labeling kit and were diluted in a series of concentrations, before mixing with CHOK1-hCD39 cells to test binding EC80 using FACS. The non-labeled antibodies were tested for their blocking efficacy to the labelled ones. Briefly, mononuclear CHOK1-hCD39 cells were prepared to 2×10⁶/ml and plated into 96 well at 50 μl/well, then mixed with antibodies gradients to final volume at 100 μl, and then equal volume of Alex488 label antibodies were added at two folds EC80 concentration. 96 well plates were incubated at 4° C. for 1 hour, and spun down and washed 3 times with 200 μl FACS buffer. The FACS analysis was performed on FACScelesta machine and data was analyzed by Flowjo software. The blocking percentages were calculated and those having above 80% competition rate were allocated into one epitope group, compared with the non-competing well (Alex488 labeled antibody only).

The competition results are shown in Table 12. Based on the competition results, the 4 anti-CD39 hybridoma antibodies (mAb14, mAb19, mAb21, mAb23) can be grouped into 4 different epitope groups, as shown in Table 11. Specifically, anti-CD39 antibodies mAb19 and mAb21 compete for highly similar epitopes, and are grouped into epitope group I, as shown in Table 11. mAb14 did not compete with any other antibody as tested, and was grouped into epitope group IV, as shown in Table 11. mAb23 showed cross-competition with mAb19 and mAb21, and was grouped into epitope group II in Table 11.

TABLE 12
Anti-CD39 hybridoma antibodies epitope binning summary.

| | mAb19- | mAb21- | mAb23- | 9-8B- | mAb14- |
|---|---|---|---|---|---|
| | Alexa488 | Alexa488 | Alexa488 | Alexa488 | Alexa488 |
| mAb19 | 96% | 83% | 91% | 6% | 23% |
| mAb21 | 98% | 90% | 97% | 20% | 24% |
| mAb23 | 98% | 93% | 100% | 98% | 86% |
| 9-8B | 19% | 16% | 100% | 98% | 55% |
| mAb14 | 6% | 10% | −26% | −11% | 98% |

3.6. Hybridoma Sequencing

RNAs were isolated from monoclonal hybridoma cells and reverse transcribed into cDNA using a commercial kit. Then the cDNA was used as templates to amplify heavy chain and light chain variable regions with the primers of Mouse Ig-Primer Set (Novagen). PCR products with correct size were collected and purified followed by ligation with a suitable plasmid vector. The ligation products were transformed into DH5a competent cells. Clones were selected and the inserted fragments were analyzed by DNA sequencing.

The variable region sequences of the hybridoma antibodies are provided herein in Table 2.

### Example 4. Chimeric Antibody Generation and Characterization

4.1. Chimeric Antibody Generation and Production

DNA encoding variable regions of 4 selected hybridoma antibodies (mAb14, mAb19, mAb21 and mAb23) was synthesized and subcloned into an expression vector where human IgG constant gene was included in advance. The vectors were transfected into mammalian cells for recombinant protein expression and the expressed antibody was purified using protein A affinity chromatography column. The resulting chimeric antibodies are referred to herein as c14, c19, c21 and c23, where the prefix “c” indicates “chimeric”, and the number indicates the hybridoma antibody clone, for example number “14” indicates that it is from the hybridoma antibody mAb14.

4.2. Chimeric Antibody Characterization

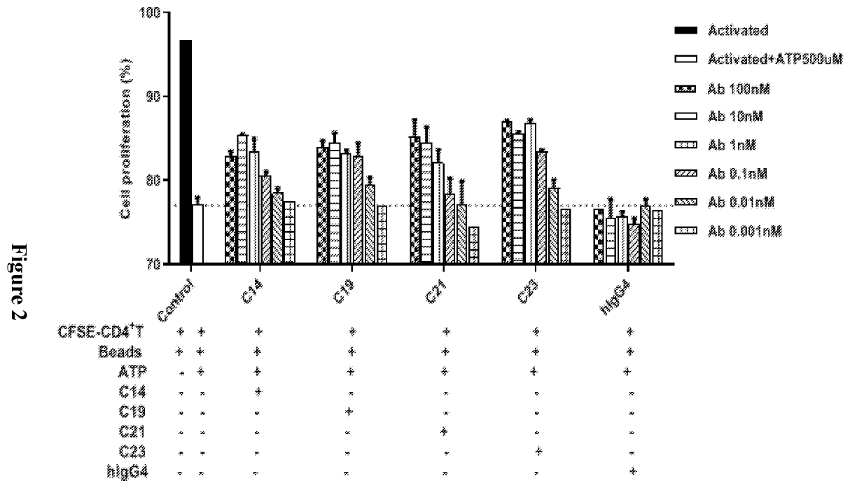

The purified 4 chimeric antibodies were tested for activity to block ATP-mediated suppression on T cell proliferation (similar as the methods described in Example 3.4). As shown in FIG. 2, anti-CD39 chimeric antibodies c14, c19, c21 and c23 blocked suppression on CD4⁺ T cell proliferation in a dose-dependent manner (at a concentration ranging from 100 nM, 10 nM, 1 nM, 0.1 nM, 0.01 nM, and 0.001 nM). CFSE-CD4⁺ T and hIgG4 were used as positive and negative controls respectively for ATP-mediated T cell proliferation.

The purified 4 chimeric antibodies were further tested for the ability to enhance ATP induced dendritic cell (DC) activation and maturation in the presence of ATP. ATP induces DC maturation through stimulation of the P2Y11 receptor on monocyte-derived dendritic cells.

Briefly, human monocytes were isolated from human healthy blood and differentiated into MoDC in presence of GM-CSF and IL-4 for 6 days. Then the differentiated MoDCs were treated with the 4 anti-CD39 chimeric antibodies with different doses and in presence of ATP for additional 24 h. DC maturation were then evaluated by analyzing CD86, CD83 and HLA-DR expression by FACS assay.

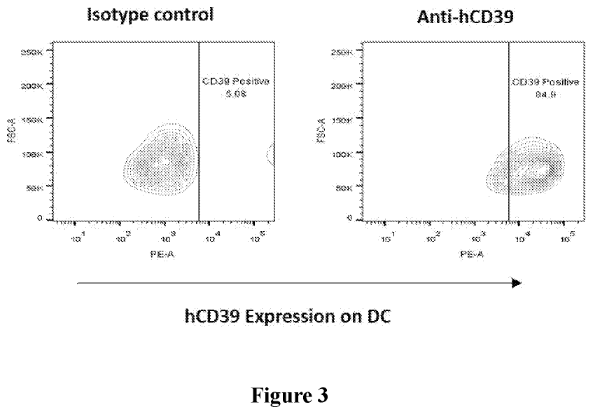

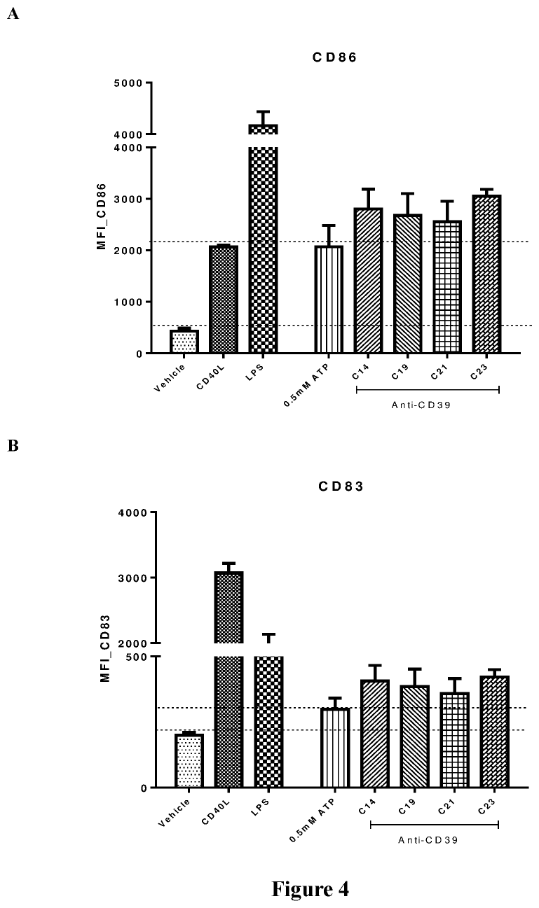

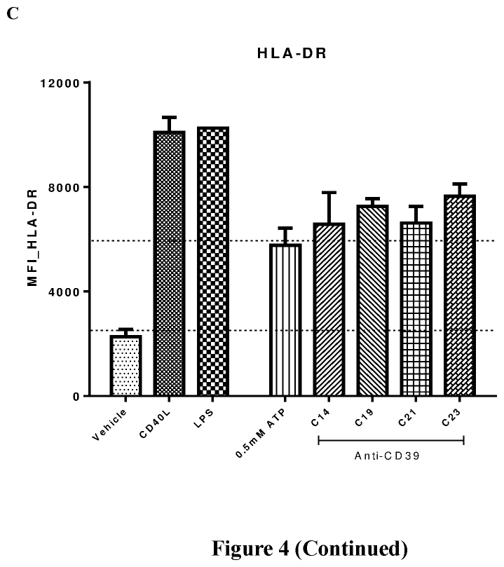

FIG. 3 showed the level of CD39 on DC surface by FACS. FIGS. 4A to 4C showed the CD86 (FIG. 4A), CD83 (FIG. 4B) and HLA-DR (FIG. 4C) expression, respectively, after the antibody treatment. The ATP induced DC maturation was shown by an increased expression of CD86, CD83, and HLA-DR, as compared with vehicle treatment. All 4 anti-CD39 antibodies c14, c19, c21 and c23 showed significant effect on enhancing ATP induced DC maturation.

The chimeric antibodies were also tested in vivo for anti-tumor activity. NOD-SCID mice were subcutaneously inoculated in the right rear flank region with tumor cells (10×10⁶) in 0.1 ml of PBS mixed with matrigel (1:1) for tumor development. The mice were randomized into groups when the mean tumor size reaches approximately 80 mm3. The treatment was initiated on the same day of randomization at 30 mg/kg, twice dosing every week. Tumor volumes were measured twice per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. Data were analyzed using two-way ANOVA by Graphpad prism.

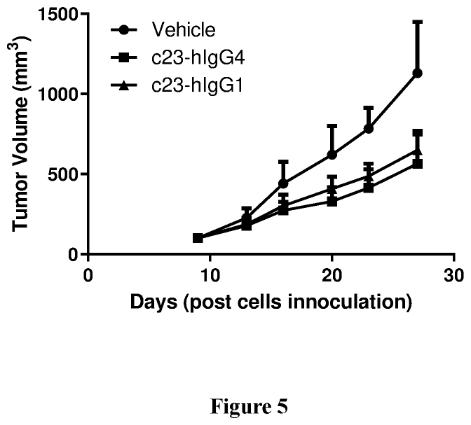

The tumor growth results of the chimeric anti-CD39 antibody c23 were shown in FIG. 5. Both the human IgG1 isotype and IgG4 isotype of c23 were obtained and tested. Both c23-hIgG4 and c23-hIgG1 chimeric antibodies demonstrated anti-tumor efficacy compared with vehicle group, and there were no significant difference identified between c23-hIgG4 and c23-hIgG1.

### Example 5. Antibody Humanization and Affinity Maturation

5.1. Humanization

Chimeric antibodies c23 and c14 were selected as the clones for humanization. Antibody sequences were aligned with human germline sequences to identify best fit model. Best matched human germline sequences were selected as the templates for humanization based on homology to the original mouse antibody sequences. The CDRs from the mouse antibody sequences were then grafted onto the templates, together with the residues to maintain the upper and central core structures of the antibodies. The optimized mutations were introduced to the framework regions to generate variants of humanized heavy chain variable regions and variants of humanized light chain variable regions, which were mixed and matched to provide multiple humanized antibody clones. After grafting and mutation, the humanized antibodies retained similar binding affinity on human CD39 expressing cells. The humanized antibodies were further evaluated by CD39 ATPase inhibition assay and in vitro immune cell activation assay. In vivo study were also conducted for some of the humanized antibodies.

A total of 31 humanized antibody clones were obtained for c23, mixing and matching 7 variants of humanized c23 heavy chain variable regions (i.e. hu23.VH_1, hu23.VH_2, hu23.VH_3, hu23.VH_4, hu23.VH_5, hu23.VH_6, and hu23.VH_7) and 7 variants of humanized c23 light chain variable regions (i.e. hu23.VL_1, hu23.VL_2, hu23.VL_3, hu23.VL_4, hu23.VL_5, hu23.VL_6, and hu23.VL_7). The 31 humanized antibody clones were designated as hu23.H1L1, hu23.H1L2, and so on, as shown in Table 9 above and Tables 13, 14 and 15 below, where the prefix “hu” indicates “humanized”, and the suffix “H1L1”, for example, denotes the serial number of the c23 humanized antibody clone, having the hu23.VH_1 variant and the hu23.VL_1 variant variable region.

TABLE 13
Heavy and light chain variable regions of humanized antibodies for c23.

| | hu23.VL_1 | hu23.VL_2 | hu23.VL_3 | hu23.VL_4 |
|---|---|---|---|---|
| | (SEQ ID NO: 61) | (SEQ ID NO: 63) | (SEQ ID NO: 65) | (SEQ ID NO: 67) |
| hu23.VH_1 | hu23.H1L1 | hu23.H1L2 | hu23.H1L3 | hu23.H1L4 |
| (SEQ ID NO: 60) | (SEQ ID NOs: | (SEQ ID NOs: | (SEQ ID NOs: | (SEQ ID NOs: |
| | 60/61) | 60/63) | 60/65) | 60/67) |
| hu23.VH_2 | hu23.H2L1 | hu23.H2L2 | hu23.H2L3 | hu23.H2L4 |
| (SEQ ID NO: 62) | (SEQ ID NOs: | (SEQ ID NOs: | (SEQ ID NOs: | (SEQ ID NOs: |
| | 62/61) | 62/63) | 62/65) | 62/67) |
| hu23.VH_3 | hu23.H3L1 | hu23.H3L2 | hu23.H3L3 | hu23.H3L4 |
| (SEQ ID NO: 64) | (SEQ ID NOs: | (SEQ ID NOs: | (SEQ ID NOs: | (SEQ ID NOs: |
| | 64/61) | 64/63) | 64/65) | 64/67) |
| hu23.VH_4 | hu23.H4L1 | hu23.H4L2 | hu23.H4L3 | hu23.H4L4 |
| (SEQ ID NO: 66) | (SEQ ID NOs: | (SEQ ID NOs: | (SEQ ID NOs: | (SEQ ID NOs: |
| | 66/61) | 66/63) | 66/65) | 66/67) |

TABLE 14
Heavy and light chain variable regions of humanized antibodies for c23.

| | hu23.VL_1 | hu23.VL_5 | hu23.VL_6 | hu23.VL_7 |
|---|---|---|---|---|
| | (SEQ ID NO: 61) | (SEQ ID NO: 143) | (SEQ ID NO: 144) | (SEQ ID NO: 145) |
| hu23.VH_1 | hu23.H1L1 | hu23.H1L5 | hu23.H1L6 | hu23.H1L7 |
| (SEQ ID NO: 60) | (SEQ ID NOs: | (SEQ ID NOs: | (SEQ ID NOs: | (SEQ ID NOs: |
| | 60/61) | 60/143) | 60/144) | 60/145) |
| hu23.VH_5 | hu23.H5L1 | hu23.H5L5 | hu23.H5L6 | hu23.H5L7 |
| (SEQ ID NO: 140) | (SEQ ID NOs: | (SEQ ID NOs: | (SEQ ID NOs: | (SEQ ID NOs: |
| | 140/61) | 140/143) | 140/144) | 140/145) |
| hu23.VH_6 | hu23.H6L1 | hu23.H6L5 | hu23.H6L6 | hu23.H6L7 |
| (SEQ ID NO: 141) | (SEQ ID NOs: | (SEQ ID NOs: | (SEQ ID NOs: | (SEQ ID NOs: |
| | 141/61) | 141/143) | 141/144) | 141/145) |
| hu23.VH_7 | hu23.H7L1 | hu23.H7L5 | hu23.H7L6 | hu23.H7L7 |
| (SEQ ID NO: 142) | (SEQ ID NOs: | (SEQ ID NOs: | (SEQ ID NOs: | (SEQ ID NOs: |
| | 142/61) | 142/143) | 142/144) | 142/145) |

TABLE 15
Heavy and light chain variable regions of humanized antibodies for c23.

| | hu23.VL_201 | hu23.VL_203 | hu23.VL_211 |
|---|---|---|---|
| | (SEQ ID NO: 111) | (SEQ ID NO: 112) | (SEQ ID NO: 63) |
| hu23.VH_201 | hu23.201 | hu23.203 | — |
| (SEQ ID NO: 146) | (SEQ ID NOs: | (SEQ ID NOs: | |
| | 146/111) | 146/112) | |
| hu23.VH_207 | hu23.207 | — | — |
| (SEQ ID NO: 147) | (SEQ ID NOs: | | |
| | 147/111) | | |
| hu23.VH_211 | — | — | hu23.211 |
| (SEQ ID NO: 39) | | | (SEQ ID NOs: |
| | | | 39/63) |

Similarly, a total of 16 humanized antibodies were obtained for c14, mixing and matching 4 variants of humanized c14 heavy chain variable regions (i.e. hu14.VH_1, hu14.VH_2, hu14.VH_3, and hu14.VH_4) and 4 variants of humanized c14 light chain variable regions (i.e. hu14.VL_1, hu14.VL_2, hu14.VL_3, and hu14.VL_4). The 16 humanized antibody clones were designated as hu14.H1L1, hu14.H1L2, and so on, as shown in below Table 16, by the same token.

TABLE 16
Heavy and light chain variable regions of 16 humanized antibodies for c14

| | hu14.VL_1 | hu14.VL_2 | hu14.VL_3 | hu14.VL_4 |
|---|---|---|---|---|
| | (SEQ ID NO: 69) | (SEQ ID NO: 71) | (SEQ ID NO: 73) | (SEQ ID NO: 75) |
| hu14.VH_1 | hu14.H1L1 | hu14.H1L2 | hu14.H1L3 | hu14.H1L4 |
| (SEQ ID NO: 68) | (SEQ ID NOs: | (SEQ ID NOs: | (SEQ ID NOs: | (SEQ ID NOs: |
| | 68/69) | 68/71) | 68/73) | 68/75) |
| hu14.VH_2 | hu14.H2L1 | hu14.H2L2 | hu14.H2L3 | hu14.H2L4 |
| (SEQ ID NO: 70) | (SEQ ID NOs: | (SEQ ID NOs: | (SEQ ID NOs: | (SEQ ID NOs: |
| | 70/69) | 70/71) | 70/73) | 70/75) |
| hu14.VH_3 | hu14.H3L1 | hu14.H3L2 | hu14.H3L3 | hu14.H3L4 |
| (SEQ ID NO: 72) | (SEQ ID NOs: | (SEQ ID NOs: | (SEQ ID NOs: | (SEQ ID NOs: |
| | 72/69) | 72/71) | 72/73) | 72/75) |
| hu14.VH_4 | hu14.H4L1 | hu14.H4L2 | hu14.H4L3 | hu14.H4L4 |
| (SEQ ID NO: 74) | (SEQ ID NOs: | (SEQ ID NOs: | (SEQ ID NOs: | (SEQ ID NOs: |
| | 74/69) | 74/71) | 74/73) | 74/75) |

Several humanized antibodies clones for c23 were also obtained by yeast display. Briefly, mouse heavy and light chain sequences were aligned with in-house database of human antibody sequences. The templates with highest homology, IGHV1-3*01 and IGKV3-11*01, were selected for heavy and light chain CDR grafting, respectively. Back mutations were identified by a high-throughput method using yeast display. Specifically, positions that contributes to CDR conformations (Vernier zone residues) were identified and a library of back mutations was created by incorporating both template and mouse residues in each position during DNA synthesis. Final candidates were identified by sequencing of top binders to human CD39 protein. Humanized antibodies for c23 obtained via yeast display are designated as hu23.201 (having a VH/VL of SEQ ID NOs:146/111), hu23.203 (having a VH/VL of SEQ ID NOs:146/112), hu23.207 (having a VH/VL of SEQ ID NOs:147/111), and hu23.211 (having a VH/VL of SEQ ID NOs:39/63).

The humanized antibodies in Tables 13, 14, 15 and 16 were recombinantly produced followed by testing for binding affinity, and were shown to be able to retain specific binding human CD39. Those having relatively higher affinity were further evaluated in functional assays including CD39 blocking assay and in vitro immune cell activation assay.

In particular, humanized antibodies hu23.H5L5, hu23.201, hu14.H1L1 and reference antibodies I394 and T895 were characterized for binding affinity against human CD39 using Biacore (GE). Briefly the antibodies to be tested were captured to CMS chip (GE) using Human Antibody Capture Kit (GE). The antigen of 6×His tagged human CD39 was serially diluted for multiple doses and injected at 30 μl/min for 180 s. Buffer flow was maintained for dissociation of 400 s. 3 M MgCl₂ was used for chip regeneration. The association and dissociation curves were fit with 1:1 binding model, and the Ka/Kd/K_{D} values for each antibody were calculated. The affinity data of the tested antibodies are summarized in Table 17 below.

TABLE 17
Binding affinity of antibodies to human
CD39 as measured by Biacore assay.

| Antibody | ka (1/Ms) | kd (1/s) | K_{D} (M) |
|---|---|---|---|
| hu23.H5L5 | 8.22E+04 | 1.60E−03 | 1.95E−08 |
| hu23.201 | 6.75E+04 | 1.62E−03 | 2.40E−08 |
| hu14.H1L1 | 9.03E+05 | 4.55E−03 | 5.03E−09 |
| I394 | 2.03E+05 | 1.26E−03 | 6.21E−09 |
| T895 | 1.33E+05 | 1.39E−01 | 1.04E−06 |

In addition, humanized antibodies hu23.H5L5 and hu14.H1L2, as well as reference antibodies I394, T895, and 9-8B were characterized for binding affinity against human CD39 using Octet assay (Creative Biolabs) according to manufacturer's manual. Briefly, the antibodies were coupled on sensors and then the sensors were dipped into CD39 gradients (start at 200 nM, with 2-fold dilution and totally 8 doses). Their binding responses were measured in real-time and results were fit globally. The affinity data of the tested antibodies are summarized in Table 18 below.

TABLE 18
Binding affinity of antibodies to human
CD39 as measured by Octet assay.

| Antibody | K_{D} (M) | kon (1/Ms) | kdis (1/s) |
|---|---|---|---|
| hu23.H5L5 | 6.87E−10 | 1.36E+05 | 9.36E−05 |
| hu14.H1L2 | 8.39E−10 | 3.89E+05 | 3.26E−04 |
| I394 | 4.54E−10 | 2.56E+05 | 1.16E−04 |
| T895 | 6.62E−09 | 6.71E+05 | 4.44E−03 |
| 9-8B | 2.02E−08 | 1.20E+05 | 2.43E−03 |

In addition, one NG motif (N55G56) which liable to deamidation was identified in HCDR2 of the humanized antibody clones for c23 antibody (e.g. hu23.H5L5). To remove the deamidation site, different mutations were introduced to N55 or G56, and it was found that N55 and G56 can be each mutated to a variety of residues, yet still retained the specific binding to human CD39. For example, it was found that when N55 was single point replaced by G, S or Q, the antibody binding affinity retained and there was no negative impact on its binding to human CD39. Similarly, when G56 was replaced by A or D, the mutant antibody also retained its specific binding and binding affinity to human CD39. Other mutations were also expected to work as well.

5.2. Binding Specificity Detection

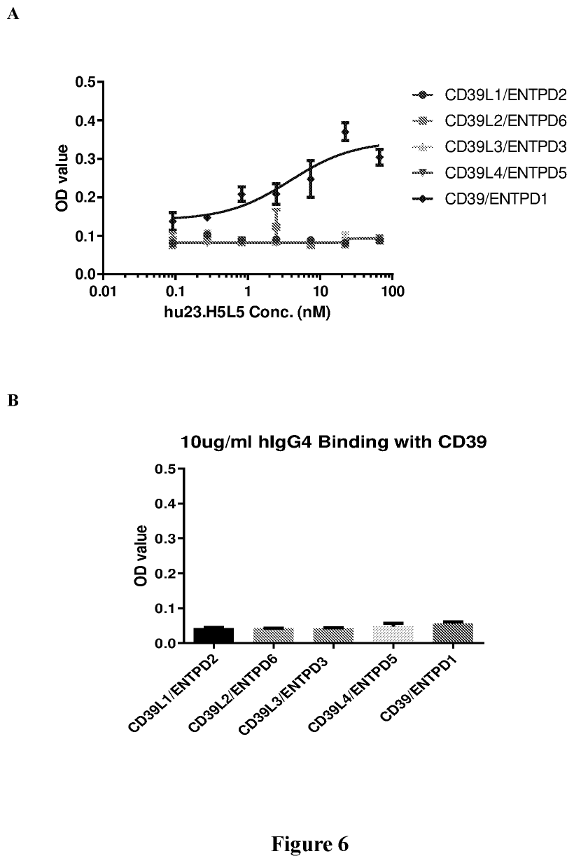

Binding specificity of the purified humanized antibody hu23.H5L5 against ENTPDase family members was detected by ELISA assay. Briefly, ENTPD1 (i.e. CD39) and ENTPD 2/3/5/6 proteins were coated on 96-well ELISA plates at 4° C. overnight, next day the ELISA plates were washed and blocked using blocking buffer (1% BSA in PBS with 0.05% Tween20) 200 μL/well for 2 hours. Then hu23.H5L5 gradients were duplicated into the wells and stained with anti-hIgG-HRP. After plate washing, the plates were developed with TMB substrate and stopped by 2N HCl. The OD450 were recorded using plate reader and platted by Graphpad Prism. The binding specificity property of hu23.H5L5 is shown in FIG. 6. It can be seen from FIG. 6A that the humanized antibody hu23.H5L5 specifically binds to human CD39, but does not bind to any of the ENTPD 2/3/5/6 proteins. FIG. 6B shows the negative control hIgG4 does not bind to any of ENTPD 1/2/3/5/6 proteins.

5.3. Humanized Antibody Characterization

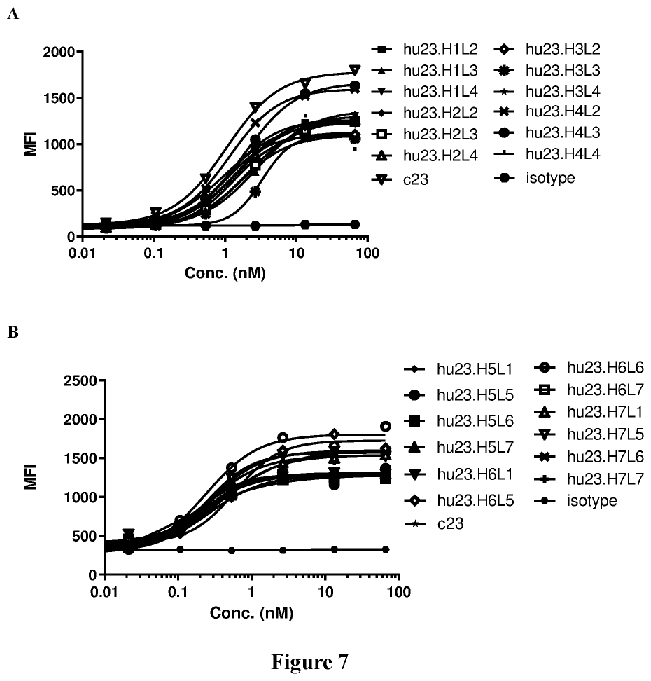

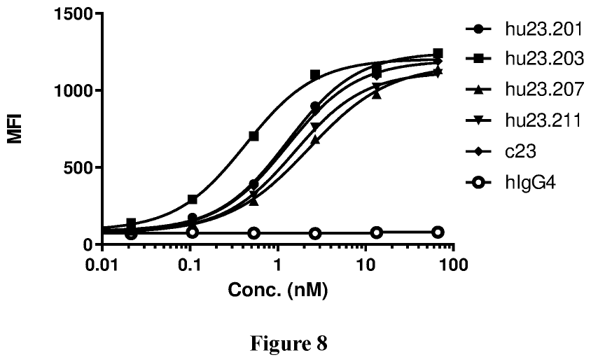

The binding affinity of the humanized antibodies for c23 was determined by FACS, using similar methods as described in Example 3.2. The c23 humanized antibody clones showing good binding affinity are listed in below Table 19 and Table 20, and also shown in FIGS. 7A, 7B and FIG. 8. EC50 is the concentration of the indicated antibodies to reach 50% of the signal in this assay.

TABLE 19
Binding activity of c23 humanized antibodies to MOLP8 cells.

| Antibody | hu23.H1L1 | hu23.H1L2 | hu23.H1L3 | hu23.H1L4 | hu23.H2L1 |
|---|---|---|---|---|---|
| EC₅₀(nM) | ~77.07 | 1.158 | 2.775 | 1.498 | 65.91 |
| Antibody | hu23.H2L2 | hu23.H2L3 | hu23.H2L4 | c23 | |
| EC₅₀(nM) | 0.979 | 2.033 | 1.46 | 1.035 | |
| Antibody | hu23.H3L1 | hu23.H3L2 | hu23.H3L3 | hu23.H3L4 | hu23.H4L1 |
| EC₅₀(nM) | ~15.40 | 1.341 | 3.29 | 1.612 | ND |
| Antibody | hu23.H4L2 | hu23.H4L3 | hu23.H4L4 | isotype | |
| EC₅₀(nM) | 1.151 | 1.868 | 1.014 | ND | |
| Antibody | hu23.H1L1 | hu23.H1L5 | hu23.H1L6 | hu23.H1L7 | hu23.H5L1 |
| EC₅₀(nM) | ~78.25 | ND | ND | ND | 0.262 |
| Antibody | hu23.H5L5 | hu23.H5L6 | hu23.H5L7 | c23 | |
| EC₅₀(nM) | 0.177 | 0.2021 | 0.179 | 0.3973 | |
| Antibody | hu23.H6L1 | hu23.H6L5 | hu23.H6L6 | hu23.H6L7 | hu23.H7L1 |
| EC₅₀(nM) | 0.2459 | 0.593 | 0.237 | 0.122 | 0.366 |
| Antibody | hu23.H7L5 | hu23.H7L6 | hu23.H7L7 | isotype | |
| EC₅₀(nM) | 0.25 | 0.271 | 0.25 | ND | |

ND: not detectable under conditions of this experiment.

TABLE 20
Binding activity of c23 humanized antibodies to MOLP8 cells.

| | Antibody | | | | | |
|---|---|---|---|---|---|---|
| | hu23.201 | hu23.203 | hu23.207 | hu23.211 | c23 | hIgG4 |
| EC₅₀ | 1.289 | 0.429 | 2.246 | 1.557 | 1.279 | ND |
| (nM) | | | | | | |

ND: not detectable under conditions of this experiment.

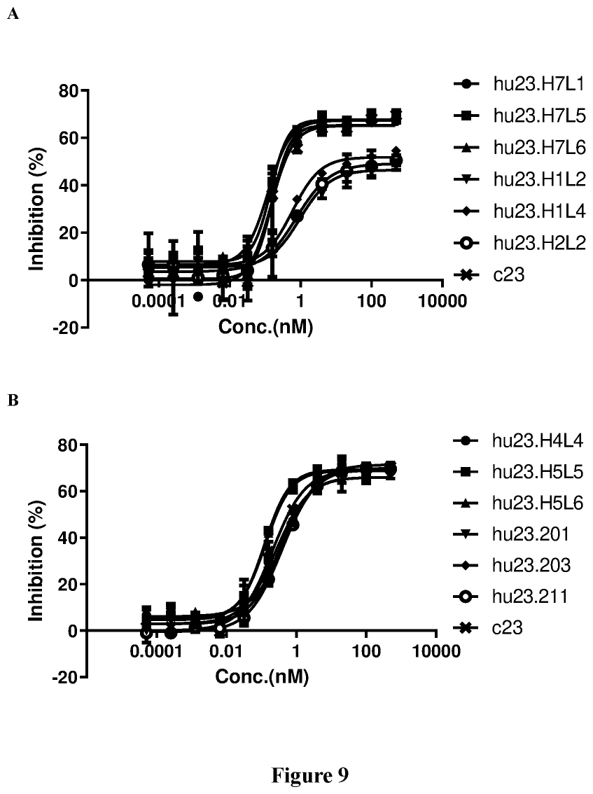

The selected humanized antibodies for c23 were tested on SK-MEL-28 cells for ATPase inhibition assay (as described in Example 3.3). FIGS. 9A and 9B show the inhibition plot of indicated antibodies, and as summarized in Table 21. Hu23.H5L5 and hu23.201 were selected for further validation.

TABLE 21
ATPase inhibition activity of c23 humanized antibodies on SK-MEL-28 cells.

| | Antibody | | | | | | |
|---|---|---|---|---|---|---|---|
| | hu23.H7L1 | hu23.H7L5 | hu23.H7L6 | hu23.H1L2 | hu23.H1L4 | hu23.H2L2 | c23 |
| IC₅₀ (nM) | 0.147 | 0.144 | 0.121 | 0.964 | 0.59 | 0.767 | 0.158 |
| | Antibody | | | | | | |
| | hu23.H4L4 | hu23.H5L5 | hu23.H5L6 | hu23.201 | hu23.203 | hu23.211 | c23 |
| IC₅₀ (nM) | 0.487 | 0.122 | 0.13 | 0.264 | 0.32 | 0.386 | 0.2092 |

The binding affinity of the humanized antibodies for c14 was determined by FACS using MOLP-8 cells expressing human CD39, using similar methods as described in Example 3.2.

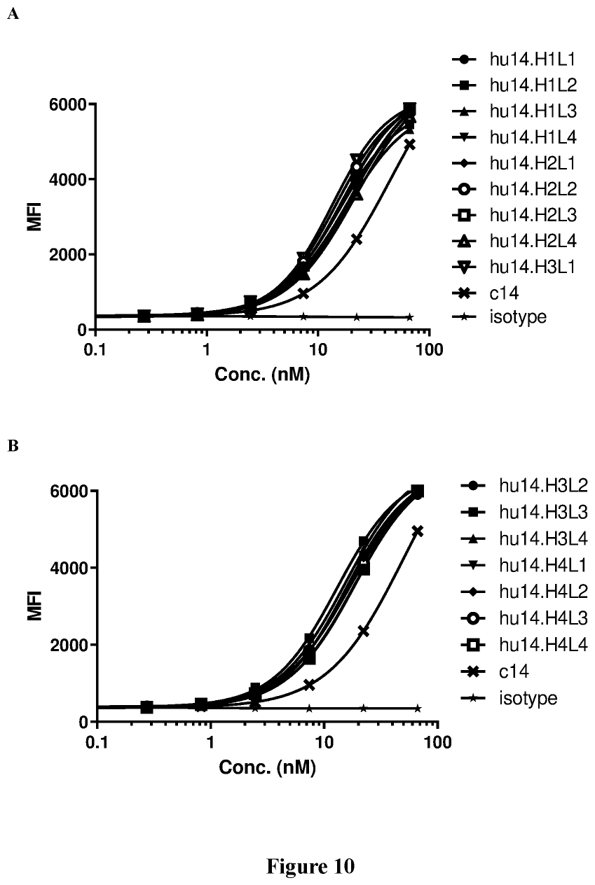

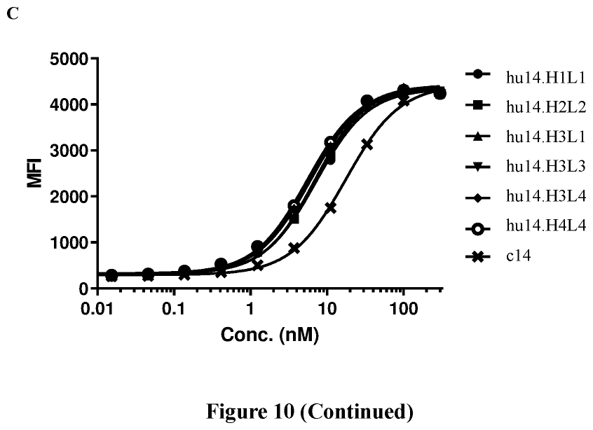

Humanized antibody clones of c14 showing good binding affinity were shown in FIGS. 10A, 10B and 10C. EC₅₀ was summarized in Table 22.

TABLE 22
Binding activity of c14 humanized antibodies to MOLP8 cells.

| | Antibody | | | | | | |
|---|---|---|---|---|---|---|---|
| | hu14.H1L1 | hu14.H2L2 | hu14.H3L1 | hu14.H3L3 | hu14.H3L4 | hu14.H4L4 | c14 |
| EC₅₀ (nM) | 7.212 | 6.908 | 5.952 | 6.088 | 6.046 | 5.459 | 17.52 |

5.4. Epitope Binning

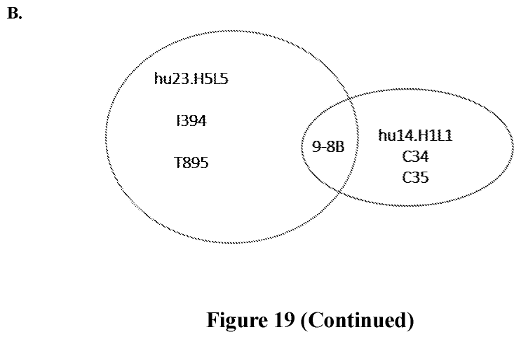

The selected humanized antibodies were tested for competitive binding (methods as described in Example 3.5). The epitope binning results of humanized antibodies hu23.H5L5 and hu14.H1L1 with reference antibodies were shown in FIG. 19A.

Based on the competition results (as shown in FIG. 19A), 2 humanized anti-CD39 antibodies hu23.H5L5 and hu14.H1L1 could be grouped into 2 different epitope groups (see FIG. 19B). Specifically, anti-CD39 antibody hu23.H5L5 competed for highly similar epitopes with reference antibodies I394, T895 and 9-8B, and was grouped into epitope group I. Besides partially competing with T895, hu14.H1L1, c34 and c35 did not compete with any other antibody as tested, and were grouped into epitope group II.

5.5. Optimized Humanized Antibody Characterization

5.5.1 CD39 Blockade by Hu23.H5L5 Improved Human T Cell Proliferation in the Presence of Extracellular ATP (eATP).

Human PBMC stimulated with anti-CD3 antibody and anti-CD28 antibody was incubated with 25 nM humanized anti-CD39 antibody hu23.H5L5 and vehicle respectively in the presence of ATP. Cell culture supernatants were harvested for detection of IL-2 and IFN-γ secretion, respectively. Proliferation of CD4⁺ T and CD8⁺ T cells was analyzed on day 5 in FACS by Cell Trace Violet dye dilution.

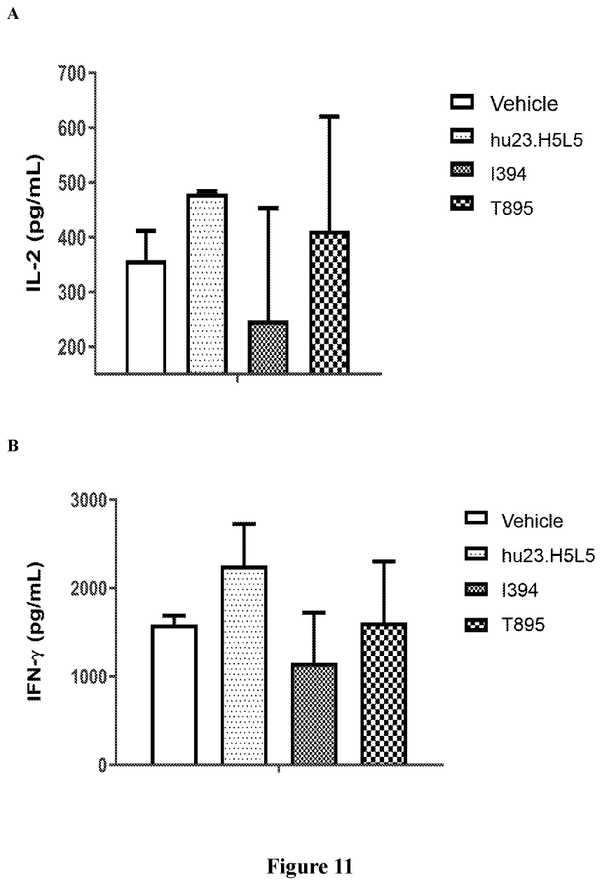

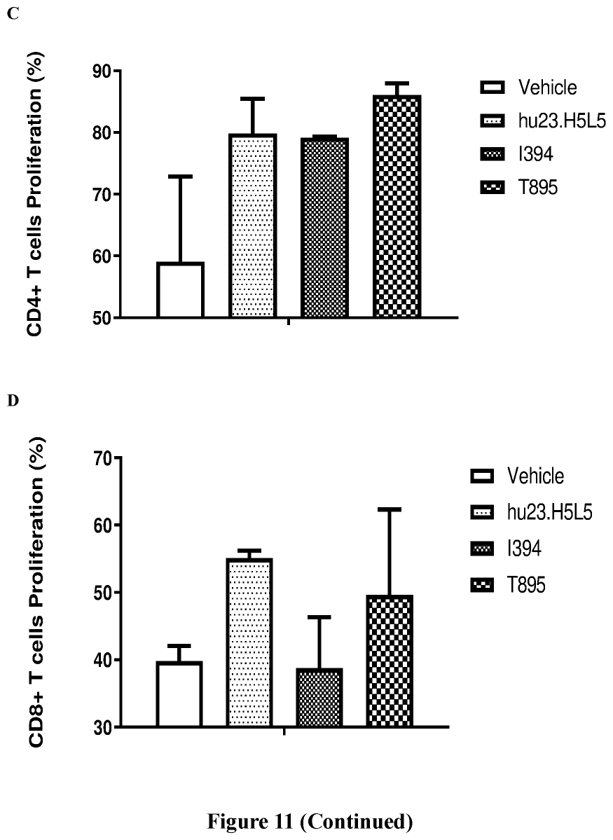

As shown in FIGS. 11A to 11D, hu23.H5L5 significantly enhanced both CD4+ and CD8⁺ T cell proliferation and activated their IL-2 and IFN-γ production at the concentration of 25 nM. As shown in FIGS. 11A, 11B and 11D, hu23.H5L5 showed significantly higher activity than I394 in enhancing T cell activation in PBMC.

Human CD8⁺ T cells were also isolated from healthy donor PBMC, then labeled with cell proliferation dye, activated with anti-CD3 antibody and anti-CD28 antibody, and treated with humanized anti-CD39 antibody hu23.H5L5 or the reference antibody I394 with different doses for a total treatment time of five days, 200 μM of ATP was added to cells on day three after the start of CD39 blockade treatment. Proliferation % of CD8⁺ T cells, % CD25+ cells and % living cells were analyzed on day 5 using flow cytometry.

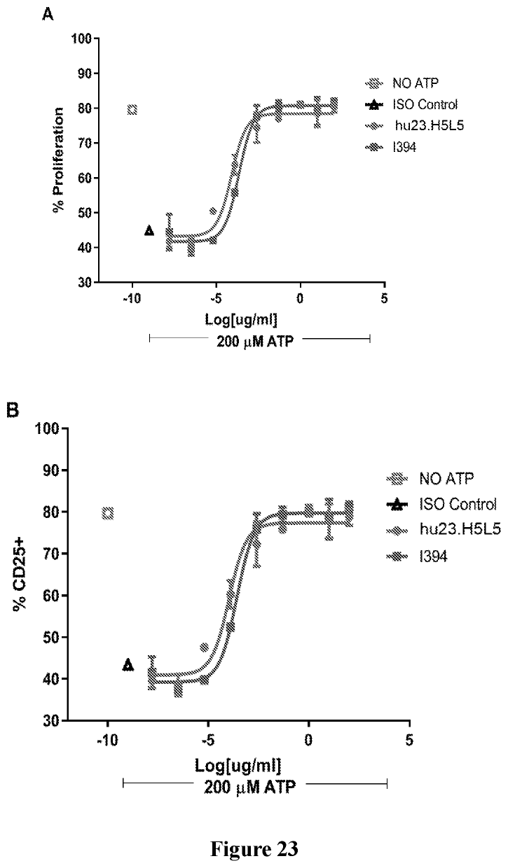

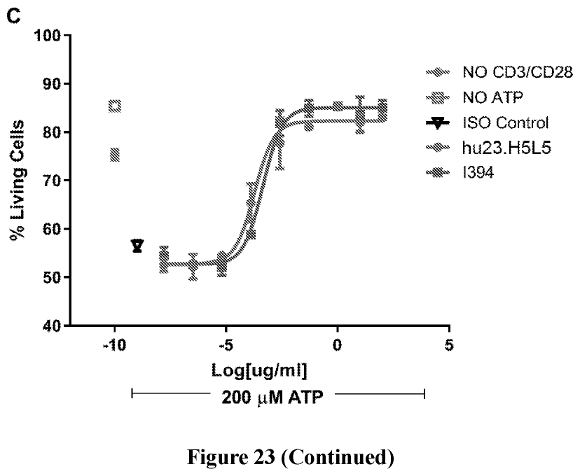

As shown in FIGS. 23A to 23C, hu23.H5L5 significantly reversed human CD8⁺ T cell proliferation which was inhibited by eATP.

Binding affinity of the humanized antibodies hu23.H5L5 and hu14.H1L1 were tested on different cells by FACS following the similar method as described in Example 3.2.

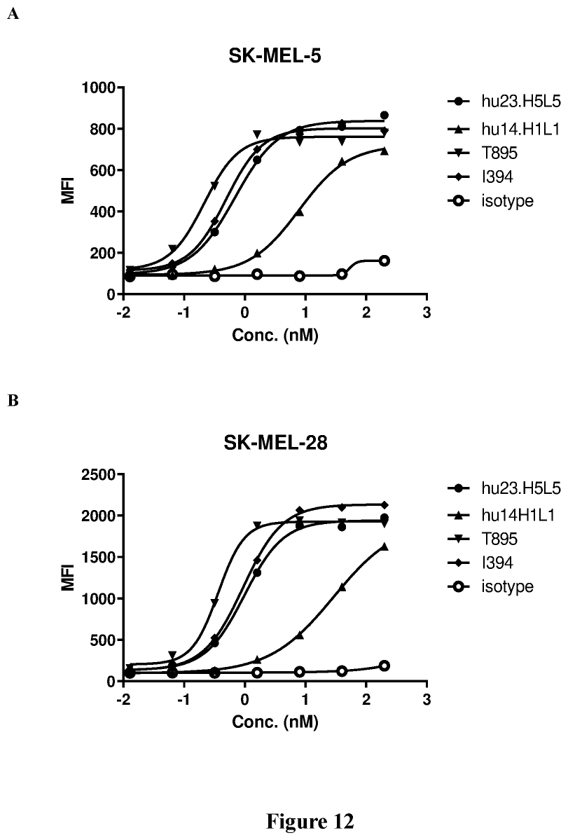

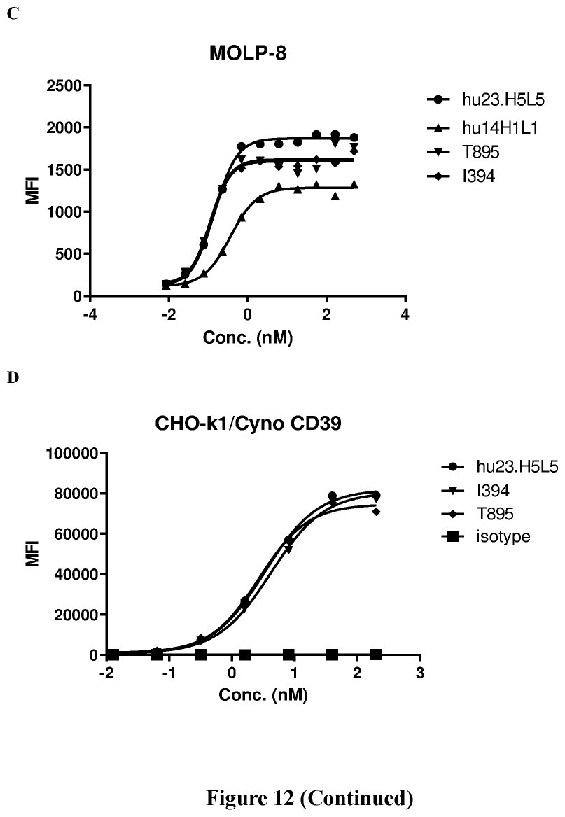

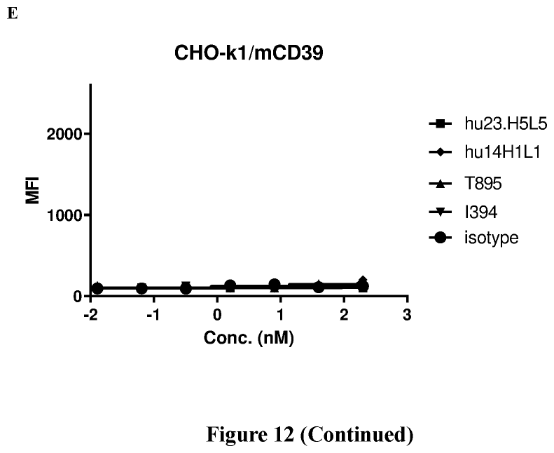

FIGS. 12A to 12E show binding affinity of antibodies hu23.H5L5 and hu14.H1L1 against SK-MEL-5 (FIG. 12A), SK-MEL-28 (FIG. 12B), MOLP-8 (FIG. 12C), CHOK1-cynoCD39 (FIG. 12D) and CHOK1-mCD39 (FIG. 12E), respectively. Reference antibodies T895 and I394 were tested in parallel as control antibodies. As shown in FIG. 12 and summarized in Table 23, both antibodies hu23.H5L5 and hu14.H1L1 bound to human and cynomolgus CD39 expressing cells in a dose-dependent manner and with similar affinity by EC₅₀ at a sub-nanomolar or nanomolar level. Neither of them recognized mouse CD39 in the FACS study. Maximum signal (mean fluorescence intensity, MFI) differed between cells for each antibody may result from their different expression level.

TABLE 23
Antibody affinity measured by FACS by EC₅₀ (nM).

| Cells | hu23.H5L5 | hu14.H1L1 | T895 | I394 |
|---|---|---|---|---|
| SK-MEL-5 | 0.69 | 7.88 | 0.21 | 0.49 |
| SK-MEL-28 | 0.99 | 29.14 | 0.36 | 0.94 |
| MOLP-8 | 0.14 | 0.35 | 0.11 | 0.11 |
| CHO-K1/cynoCD39 | 3.375 | ND | 4.192 | 2.708 |
| CHO-K1/mCD39 | ND | ND | ND | ND |

ND: not detectable under conditions of this experiment.

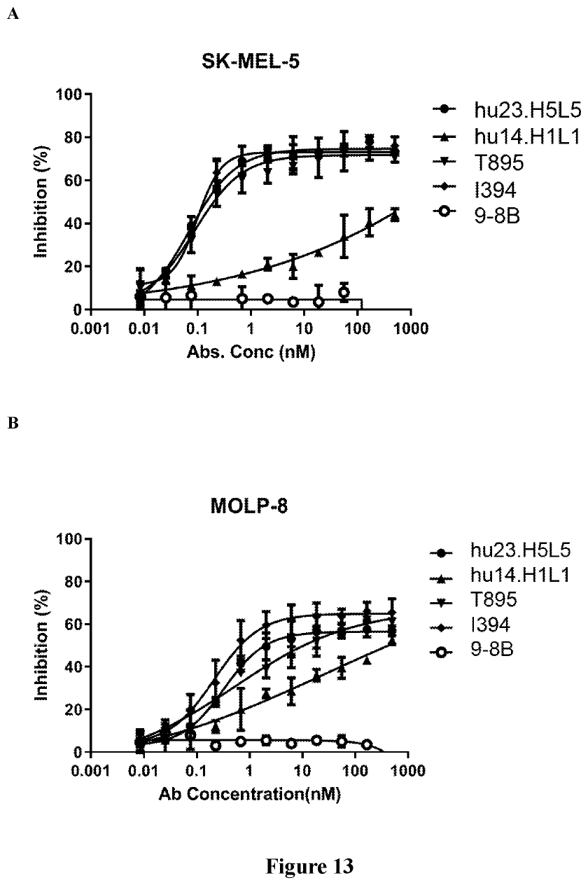

FIG. 13 shows that hu23.H5L5 blocked CD39 ATPase activity on SK-MEL-5 cells (FIG. 13A) or MOLP-8 cells (FIG. 13B), similar to the reference antibodies T895 and I394 (method as described in Example 3.3). Results were summarized in Table 24.

hu23.H5L5 showed 70 μM enzymatic blocking IC 50 on SK-MEL-5 cells and 330 μM on MOLP-8 cells which were similar or slightly better than the reference antibodies T895 and I394. 9-8B identified as a non-blocker in this assay.

TABLE 24
ATPase activity inhibition (IC₅₀) of humanized antibodies (nM)

| IC₅₀ (nM) | hu23.H5L5 | T895 | I394 | 9-8B |
|---|---|---|---|---|
| SK-MEL-5 | 0.07 | 0.09 | 0.10 | ND |
| MOLP-8 | 0.33 | 0.51 | 0.21 | ND |

ND: not detectable under conditions of this experiment

5.5.2 CD39 Blockade by Hu23.H5L5 Enhanced ATP-Mediated Monocytes Activation.

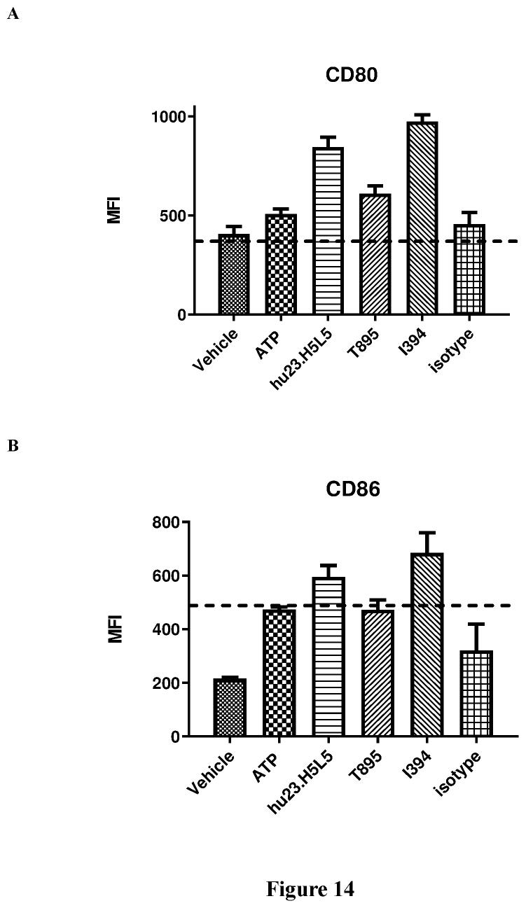

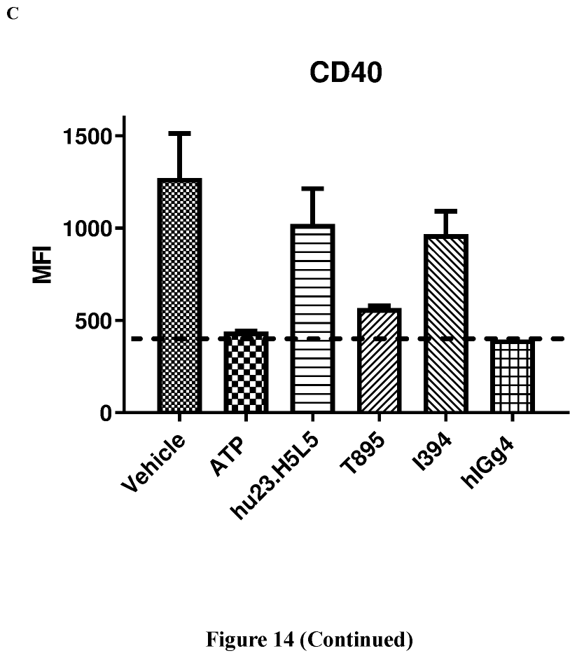

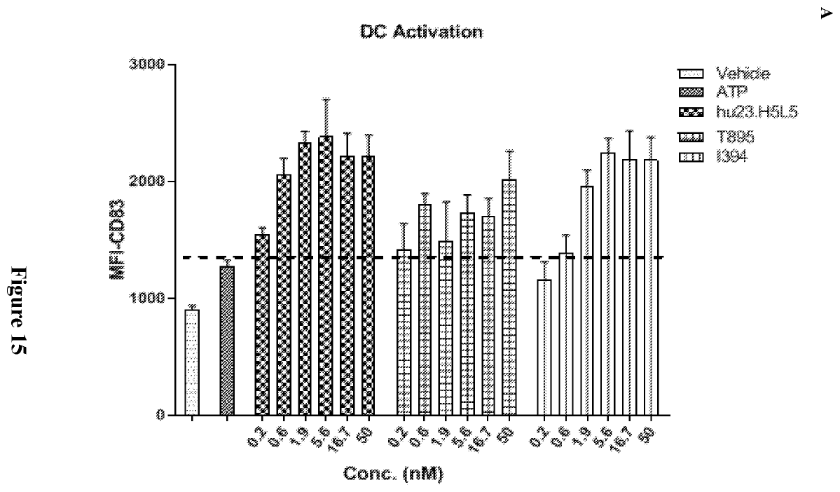

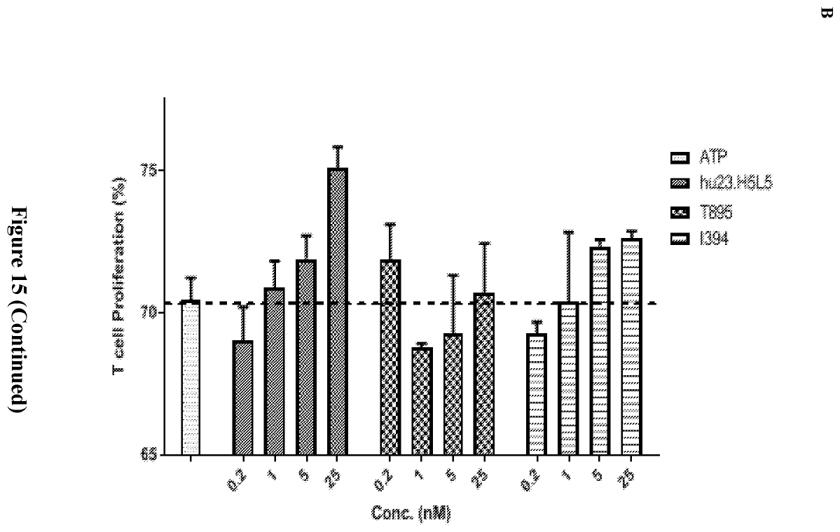

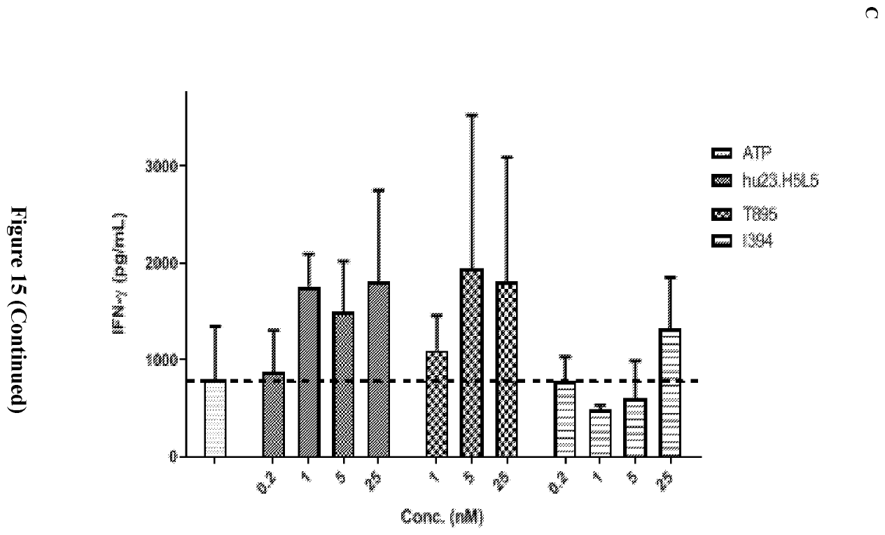

The humanized antibody hu23.H5L5 was also tested in ATP-mediated monocyte activation assay. ATP-mediated pro-inflammatory activity has an important role in regulating the function of multiple immune cell types, including monocyte. To evaluate whether CD39 blockade could enhance ATP-mediated monocytes activation, human monocytes were purified from human healthy blood, and then incubated in the presence of ATP with anti-CD39 antibodies at various concentrations ranging from 0.2 nM to 100 nM. Hu23.H5L5 was shown to be effective in inducing monocyte activation at 0.2 nM, i.e., the lowest concentration tested. Monocyte activation was assessed by analyzing CD80 (FIG. 14A), CD86 (FIG. 14B) and CD40 (FIG. 14C) expression by FACS assay (the concentration of hu23.H5L5 is 50 nM). Reference anti-CD39 antibodies I394 and T895 were used as control, hIgG4 was used as an isotype control.

Results are shown in FIG. 14. Stimulation of ATP alone demonstrated upregulated expression of CD80 and CD86, indicating monocytes activation. Anti-CD39 humanized antibody hu23.H5L5 further enhanced the ATP-mediated monocytes activation, as evidenced by the upregulation of CD80, CD86, and CD40, at a level comparable to that of the reference antibody I394. Reference antibody T895 didn't show significant effect on ATP induced activated monocytes.

5.5.3 CD39 Blockade by Hu23.H5L5 Enhanced ATP-Mediated DC Activation.

The selected humanized antibody hu23.H5L5 was also tested in ATP-mediated DC activation assay (following similar methods described in Example 4.2). Briefly, DC maturation were evaluated by analyzing CD83 expression by FACS assay. ATP induced DC maturation by showing an increased expression of CD83 (FIG. 15A). Hu23.H5L5 increased CD83 expression in a dose-dependent manner, starting from a level as low as 0.2 nM, and significantly increased CD83 expression at an antibody level of 0.6 nM. This is more potent than any of the reference antibodies T895 and I394.

To further assess the consequential effect of ATP-mediated DC activation on T cells activation, ATP-activated DC were washed and then incubated with allogenic T cells for a mixed lymphocytes reaction (MLR). T cells proliferation (FIG. 15B) and IFN-γ production from activated T cells were analyzed (FIG. 15C).

In comparison with the reference antibodies I394 and T895, anti-CD39 antibody hu23.H5L5 showed dose-dependent and significant effect on enhancing ATP induced DC maturation, reference I394 showed similar but a slightly weaker activity, while the effect of T895 was very mild. Consistently, as shown in FIGS. 15B and 15C, the enhanced ATP-mediated MoDC maturation by anti-CD39 blocking antibody hu23.H5L5 resulted in the higher T cells proliferation and IFN-γ production in the MLR assay.

5.5.4 CD39 Blockade by Hu23.H5L5 Promoted Human Macrophage IL1β Release Induced by LPS Stimulation.

Human CD14⁺ T cells were isolated from human healthy PBMC, the enriched CD14+ monocytes were then seeded at the density of 2×10⁶ per well in a 6-well plate and cultured with 100 ng/mL human GM-CSF for 6 days to generate Ml-like macrophage. In vitro differentiated macrophage were treated with hu23.H5L5 or reference antibody I394 in increasing doses for 1 h and, subsequently, stimulated with ng/mL LPS for 3 hours before addition of 800 μM ATP for 2 hours. IL-1I3 in cell culture supernatants was quantified by ELISA.

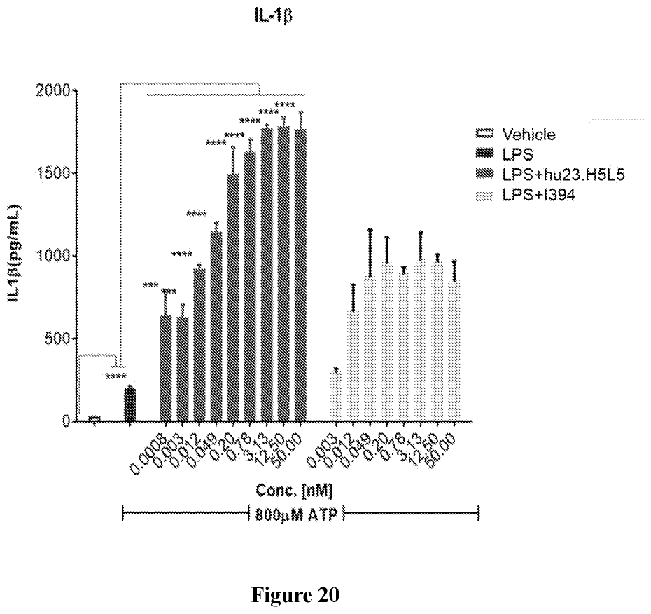

Results are shown in FIG. 20. Asterisks indicate significant differences between the respective conditions. As shown in FIG. 20, hu23.H5L5 significantly promoted human macrophage IL1β release induced by LPS stimulation, and hu23.H5L5 showed significantly higher activity than reference antibody I394 in promoting human macrophage IL1β release induced by LPS stimulation.

5.6. In Vivo Study

The effect of humanized antibodies hu23.H5L5 and hu14.H1L1 were determined on MOLP-8 xenograft mice according to methods described in Example 4.2.

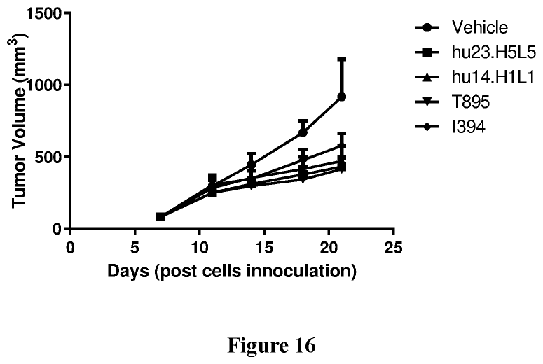

Results are shown in FIG. 16, all of the anti-CD39 antibodies inhibited tumor growth compared with vehicle group. The efficacy observed for I394 was slightly weaker than the other antibodies including hu23.H5L5 and hu14.H1L1.

The anti-tumor efficacy of humanized antibody hu23.H5L5 was also tested in vivo in PBMC adoption animal model (NCG mice, inoculated with MOLP-8 cells, by testing a range of different dosages (0.03 mg/kg, 0.3 mg/kg, 3 mg/kg, mg/kg, 30 mg/kg, i.p., BIW×6 doses), according to the methods described in Example 4.2.

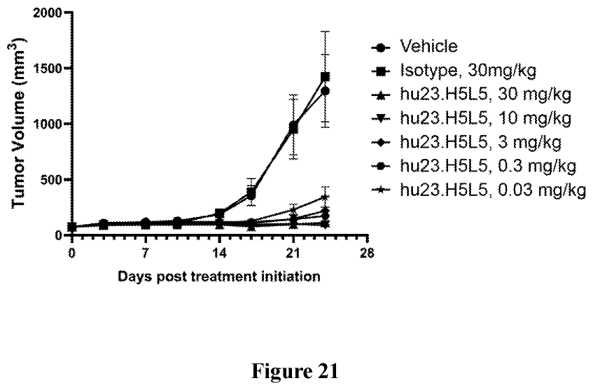

Results are shown in FIG. 21. As shown by FIG. 21, the humanized antibody hu23.H5L5 potently inhibits tumor growth at all tested dosages.

We also determined whether the anti-tumor efficacy of the anti-CD39 antibodies were dependent on NK cells or macrophage cells. The NK depleting treatment of anti-asialo-GM1 was initiated on day 7 at 20 μl/mouse intraperitoneally, once every 5 days. The macrophage depleting treatment of clodronate liposome was also initiated on day 7 and day 9 at 200 μl/mouse intravenously, once per week. Blood samples analysis data demonstrated mononuclear phagocytic cells or NK were significantly removed by the reagent.

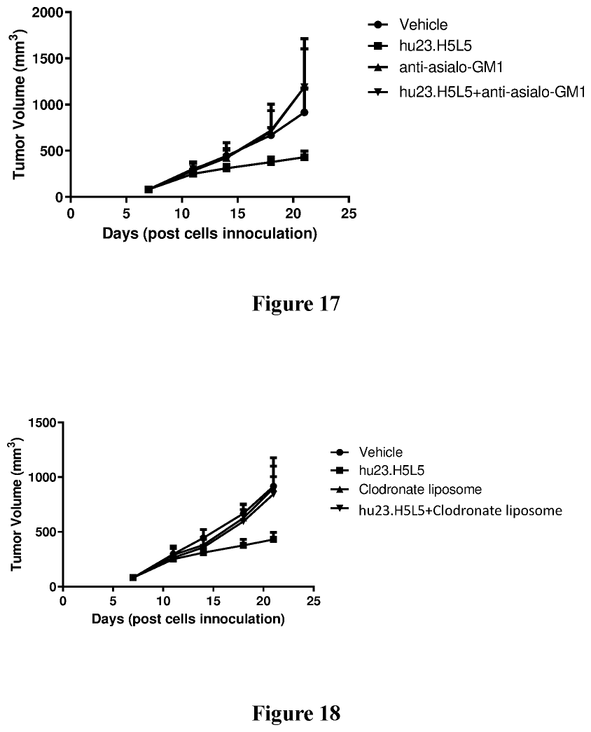

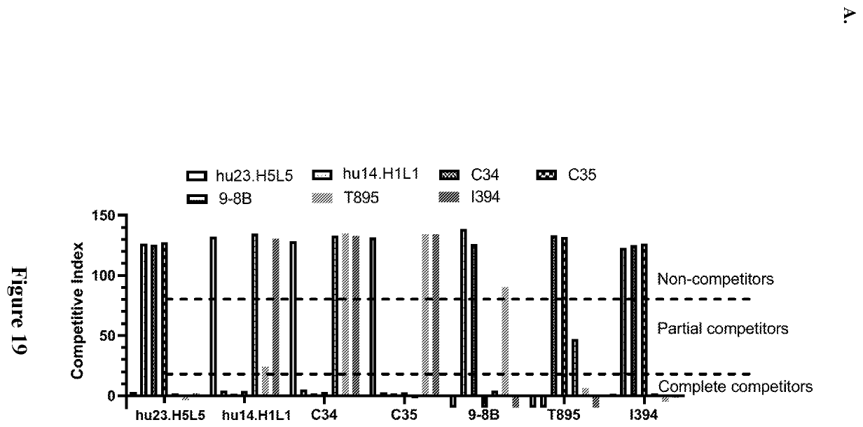

In the models where NK (FIG. 17) or macrophage (FIG. 18) cells were depleted, the tumor growth inhibition effect of hu23.H5L5 was abolished, suggesting that the anti-tumor effects of the anti-CD39 antibody was dependent on NK cells and macrophages.

Specifically, as shown in FIG. 17, anti-asialo-GM1 slightly enhanced tumor growth at late stage compared with vehicle. And compared with hu23.H5L5 treated group, its combination with anti-asialo-GM1 completely abolished hu23.H5L5 tumor growth inhibition efficacy. As shown in FIG. 18, clodronate liposome had no effect on tumor growth compared with vehicle. However, clodronate liposome treatment completely abolished hu23.H5L5's tumor growth inhibition efficacy.

### Example 6. Epitope Mapping

To define the epitope of anti-CD39 antibodies, CD39 mutants were designed and defined by substitutions of amino acids exposed at the molecular surface over the surface of human CD39. Mutants were cloned into an expression vector which fused a C-terminal EGFP sequence and transfected in HEK-293F cells, as shown in Table 25 below. The targeted amino acid mutations are shown using numbering of UniProtKB—P49961 (ENTP1_HUMAN), which is the wild-type amino acid sequence of human CD39, and shown as SEQ ID NO: 162 herein. For example, V77G means that valine at position 77 of SEQ ID NO: 162 is replaced by glycine.

TABLE 25
Human CD39 Mutants

| Mutants ID | Substitutions |
|---|---|
| KW27-1 | V77G, H79Q, Q444K, G445D |
| KW27-2 | V81S, E82A, R111A, V115A |
| KW27-3 | E110A, R113T, E114A |
| KW27-4 | R118A, S119A, Q120K, Q122H, E123A |
| KW27-5 | D150A, E153S, R154A, S157K, N158A, L278F |
| KW27-6 | Q96A, N99A, E143A, R147E |
| KW27-7 | K188R, 190-206(SQKTRWFSIVPYETNNQ) |
| | substituted by KTPGGS |
| KW27-8 | A273S, N275A, I277S, R279A |
| KW27-9 | S294A, K298G, K303A, E306A, T308K, Q312A |
| KW27-10 | K288E, K289A, V290A, E315R |
| KW27-11 | Q354A, D356S, E435A, H436Q |
| KW27-12 | H428A, T430A, A431D, D432A |
| KW27-13 | N371K, L372K, E375A, K376G, V377S |
| KW27-14 | K388N, Q392K, P393S, E396A |
| KW27-15 | A402P, G403A, K405A, E406A |
| KW27-16 | K5A, E100A, D107A |
| KW27-17 | Q323A, Q324A, Q327A, E331K |
| KW27-18 | N334A, S336A, Y337G, N346A |
| KW27-19 | Q228A, I230S, D234A, Q238A |

| KW27-20 | R138A, M139A, E142K |
|---|---|
| KW27 | SEQ ID NO: 162 (wild-type human CD39) |
| | MEDTKESNVK TFCSKNILAI LGESSIIAVI ALLAVGLTQN |
| | KALPENVKYG IVLDAGSSHT SLYIYKWPAE KENDTGVVHQ |
| | VEECRVKGPG ISKFVQKVNE IGIYLTDCME RAREVIPRSQ |
| | HQETPVYLGA TAGMRLLRME SEELADRVLD VVERSLSNYP |
| | FDFQGARIIT GQEEGAYGWI TINYLLGKFS QKTRWFSIVP |
| | YETNNQETFG ALDLGGASTQ VTFVPQNQTI ESPDNALQFR |
| | LYGKDYNVYT HSFLCYGKDQ ALWQKLAKDI QVASNEILRD |
| | PCFHPGYKKV VNVSDLYKTP CTKRFEMTLP FQQFEIQGIG |
| | NYQQCHQSIL ELFNTSYCPY SQCAFNGIFL PPLQGDFGAF |
| | SAFYFVMKFL NLTSEKVSQE KVTEMMKKFC AQPWEEIKTS |
| | YAGVKEKYLS EYCFSGTYIL SLLLQGYHFT ADSWEHIHFI |
| | GKIQGSDAGW TLGYMLNLIN MIPAEQPLST PLSHSTYVEL |
| | MVLESLVLFT VAIIGLLIFH KPSYFWKDMV |

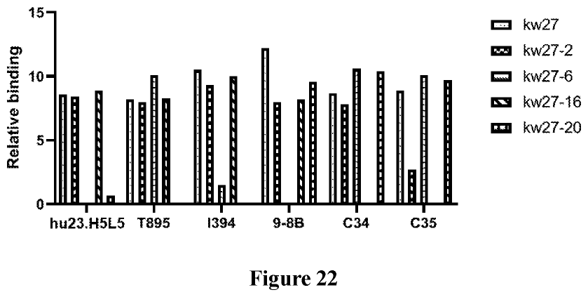

Briefly, the human CD39 mutants were generated by gene synthesis and then cloned into an expression vector pCMV3-GFPSpark. The vectors containing the validated mutated sequences were prepared and transfected into HEK293F cells. Three days post transfection, the cells were collected to testing EGFP for transgene expression. A range of dosages of antibodies (start from 100 nM, 3-folds dilution, 11 points) were tested on the 20 generated mutants and stained by AlexFluor647 labelled anti-hIgG by FACS. Antibody binding was descripted as relative binding which is derived from AlexFluor647 intensity divided by GFP intensity. The results were shown in FIG. 22.

As shown in FIG. 22, the humanized antibody hu23.H5L5 lost binding to mutant KW27-6 and KW27-20, but not to the other mutants. Mutant KW27-6 contains amino acid substitutions at residues Q96, N99, E143 and R147, indicating that one or more, or all of the residues of the mutant are important to the core epitope of hu23.H5L5; Mutant KW27-20 contains amino acid substitutions at residue R138, M139 and E142, indicating that one or more, or all of the residues of the mutant are also important to the core epitope of hu23.H5L5.

As shown in FIG. 22, the chimeric antibody c34 lost binding to mutant KW27-16, but not to any other mutants. Mutant KW27-16 contains amino acid substitutions at residues K5, E100 and D107, indicating that one or more, or all of the residues of the mutant are important to the core epitope of c34.

As shown in FIG. 22, the chimeric antibody c35 lost binding to mutant KW27-2, but not to any other mutants. Mutant KW27-2 contains amino acid substitutions at residues V81, E82, R111 and V115, indicating that one or more, or all of the residues of the mutant are important to the core epitope of c35.

As shown in FIG. 22, the reference antibody T895 lost binding to mutant KW27-20, but not to any other mutants. Mutant KW27-20 contains amino acid substitutions at residue R138, M139 and E142 indicating that one or more, or all of the residues of the mutant are important to the core epitope of T895.

As shown in FIG. 22, the reference antibody I394 lost binding to mutant KW27-6 and KW27-20, but not to the other mutants. Mutant KW27-6 contains amino acid substitutions at residues Q96, N99, E143 and R147, indicating that one or more, or all of the residues of the mutant are important to the core epitope of I394; Mutant KW27-20 contains amino acid substitutions at residues R138, M139 and E142, indicating that one or more, or all of the residues of the mutant are also important to the core epitope of I394.

As shown in FIG. 22, the reference antibody 9-8B lost binding to mutant KW27-6, but not to any other mutants. Mutant KW27-6 contains amino acid substitutions at residues Q96, N99, E143 and R147, indicating that one or more, or all of the residues of the mutant are important to the core epitope of 9-8B.

### Example 7. Anti-CD39/TGFβ Trap Construction and Expression

Anti-CD39/TGFβ Trap molecule is constructed as an anti-CD39 antibody moiety linked to TGFβ receptor II ECD (TGFβRII ECD) at the N-terminus or C-terminus of the heavy and/or light chains of the anti-CD39 antibody moiety. A flexible (Gly 4 Ser) 3 linker was genetically linked to the N-terminus of the TGFβRII ECD. Several Anti-CD39/TGFβ Trap molecules were constructed with changed TGFβRII ECD molar ratios and positions on the anti-CD39 antibody moiety, and their schematic drawings were shown in FIGS. 24A-G, respectively. Anti-CD39/TGFβ Trap molecules comprising an anti-CD39 antibody moiety linked to TGFβ receptor I ECD (TGFβRI ECD) or TGFβ receptor III ECD (TGFβRIII ECD) may be constructed by a similar way, and were not shown herein.

The anti-CD39/TGFβ Trap molecule ES014-1 comprising one anti-CD39 antibody moiety (i.e. hu23.H5L5) and two TGFβRII ECDs (i.e. SEQ ID NO: 164), wherein one TGFβRII ECD is linked to the anti-CD39 antibody moiety at the C-terminus of each of the heavy chain constant region (FIG. 24A).

The anti-CD39/TGFβ Trap molecule ES014-2 comprising one anti-CD39 antibody moiety (i.e. hu23.H5L5) and four TGFβRII ECDs (i.e. SEQ ID NO: 164), wherein two TGFβRII ECDs are linked to the anti-CD39 antibody moiety at the C-terminus of each of the heavy chain constant region (FIG. 24B).

The anti-CD39/TGFβ Trap molecule ES014-3 comprising one anti-CD39 antibody moiety (i.e. hu23.H5L5) and four TGFβII ECDs (i.e. SEQ ID NO: 164), wherein two TGFβRII ECDs are linked to the anti-CD39 antibody moiety at the N-terminus of each of the heavy chain variable region (FIG. 24C).

The anti-CD39/TGFβ Trap molecule ES014-4 comprising one anti-CD39 antibody moiety (i.e. hu23.H5L5) and four TGFβII ECDs (i.e. SEQ ID NO: 164), wherein two TGFβRII ECDs are linked to the anti-CD39 antibody moiety at the N-terminus of each of the light chain variable region (FIG. 24D).

The anti-CD39/TGFβ Trap molecule ES014-5 comprising one anti-CD39 antibody moiety (i.e. hu23.H5L5) and four TGFβII ECDs (i.e. SEQ ID NO: 164), wherein one TGFβII ECD is linked to the anti-CD39 antibody moiety at the N-terminus of each of the heavy chain variable region, and one TGFβII ECD is linked to the anti-CD39 antibody moiety at the N-terminus of each of the light chain variable region (FIG. 24E).

The anti-CD39/TGFβ Trap molecule ES014-6 comprising one anti-CD39 antibody moiety (i.e. hu23.H5L5) and four TGFβII ECDs (i.e. SEQ ID NO: 164), wherein two TGFβRII ECDs are linked to the anti-CD39 antibody moiety at the C-terminus of each of the light chain constant region (FIG. 24F).

The anti-CD39/TGFβ Trap molecule ES014-7 comprising one anti-CD39 antibody moiety (i.e. hu23.H5L5) and six TGFβII ECDs (i.e. SEQ ID NO: 164), wherein one TGFβRII ECD is linked to the anti-CD39 antibody moiety at the C-terminus of each of the heavy chain constant region, and two TGFβRII ECDs are linked to the anti-CD39 antibody moiety at the C-terminus of each of the light chain constant region (FIG. 24G).

For expression, the DNA encoding the light chain and the heavy chain in either the same expression vector or separate expression vectors were used to transfect CHO cell for transfection. The culture media were harvested and the fusion protein was purified by Protein A Sepharose column.

### Example 8. Binding of the Anti-CD39/TGFβ Trap Molecules to TGFβ by ELISA

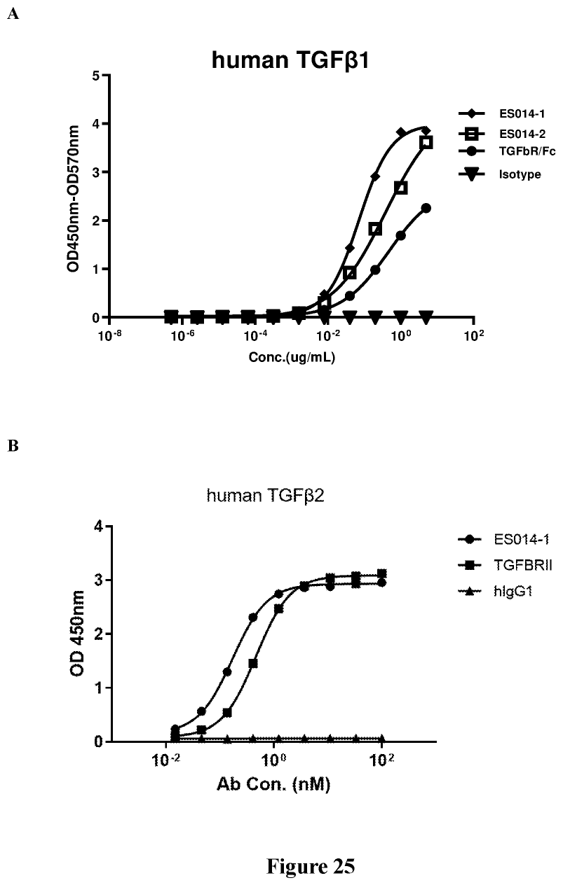

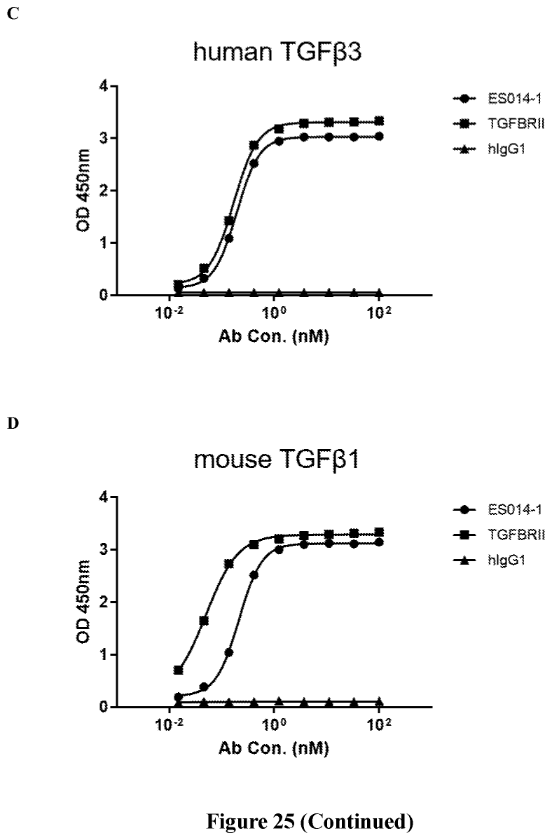

To determine the binding ability and specificity of the anti-CD39/TGFβ Trap molecules, ELISA assays were conducted using human TGFβ1, human TGFβ2, human TGFβ3 as well as mouse TGFβ1. The tested antigens were coated on NUNC 96-well immunoplate at the concentration of 1 μg/ml. Binding with increasing concentrations of anti-CD39/TGFβ Trap molecules was measured with anti-human Fc antibody horseradish peroxidase conjugate diluted in PBT buffer, then developed with TMB substrate. Soluble TGFβ trap was used as control. As shown in FIGS. 25A-25C, the anti-CD39/TGFβ Trap molecules ES014-1 and ES014-2 bind to all three TGFβ homologues: human TGFβ1, TGFβ2 and TGFβ3. The binding assay results of the other tested anti-CD39/TGFβ Trap molecules (including ES014-3, ES014-4, ES014-5, ES014-6, ES014-7) were similar and not shown herein. EC₅₀ for human TGFβ1 was listed in Table 26 below. Moreover, the anti-CD39/TGFβ Trap molecule ES014-1 binds to mouse TGFβ1 with similar affinity as for human TGFβ1 (FIG. 25D).

TABLE 26
Binding EC₅₀ of anti-CD39/TGFβ Trap
molecules to human TGFβ1.

| Anti-CD39/TGFβ Trap | ES014-1 | ES014-2 | ES014-3 | ES014-4 |
|---|---|---|---|---|
| EC₅₀ (nM) | 0.26 | 0.26 | 0.24 | 0.11 |
| Anti-CD39/TGFβ Trap | ES014-5 | ES014-6 | ES014-7 | TGFβRII |
| EC₅₀ (nM) | 0.09 | 0.07 | 0.09 | 0.017 |

### Example 9. TGFβ and TGFβR Blocking Assay

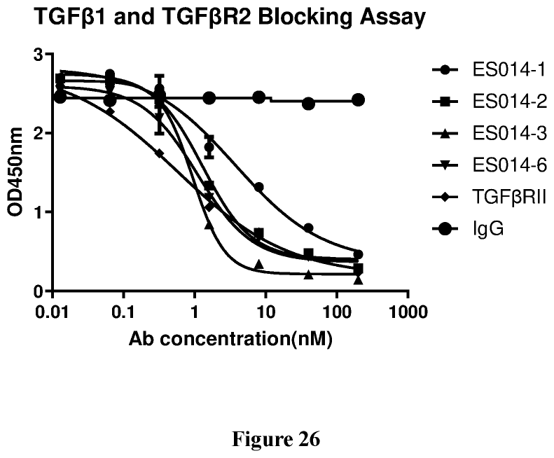

For blocking assay, TGFβ peptide (TGFβ1) was coated on microplates. A serial dilution of purified antibodies was incubated with recombinant TGFβRII-His protein (SinoBiological) for 1 h in TGFβ1-coated plates. After wash, the remaining TGFβRII-His was detected by anti-His-HRP conjugated secondary antibody. The values of absorbance at 450 nm were read on a microtiter plate reader (Molecular Devices Corp) for the quantification of TGFβRII-His binding to TGFβ1. All the tested anti-CD39/TGFβ Trap molecules (i.e. ES014-1, ES014-2, ES014-3, ES014-6) could effectively block human TGFβ1 binding to the TGFβ receptor TGFβRII (FIG. 26, Soluble TGFβ trap (i.e. TGFβRII) was used as control). IC 50 values of the anti-CD39/TGFβ Trap molecules were analyzed using GraphPad Prism. The anti-CD39/TGFβ Trap molecules with four copies of TGFβRII ECDs, such as ES014-2, ES014-3 and ES014-6, are more potent than the anti-CD39/TGFβ Trap molecules with two copies of TGFβRII ECDs like ES014-1 (Table 27).

TABLE 27
Blocking IC₅₀ of anti-CD39/TGFβ Trap molecules
to human TGFβ1 and TGFβ1RII.

| Anti-CD39/TGFβ Trap | ES014-1 | ES014-2 | ES014-3 | ES014-6 | TGFβRII |
|---|---|---|---|---|---|
| IC₅₀ (nM) | 3.73 | 1.29 | 0.93 | 1.09 | 0.58 |

### Example 10. Binding of Anti-CD39/TGFβ Trap Molecules to CD39 by FACS

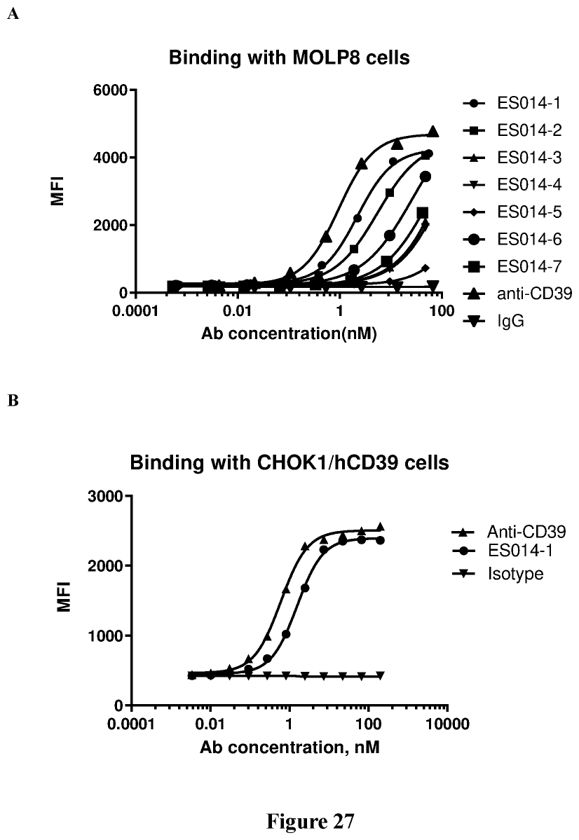

Approximately 100,000 MOLP-8 myeloma cells overexpressing CD39 were washed with wash buffer and incubated with 100 μl serial dilution of anti-CD39/TGFβ Trap molecules for 30 minutes on ice. Cells were then washed twice with wash buffer and incubated with 100 μl of anti-human Fc-PE for 30 minutes on ice. Cells were then washed twice with wash buffer and analyzed on a FACS Canto II analyzer (BD Biosciences). As shown in FIG. 27A, all of the tested anti-CD39/TGFβ Trap molecules (e.g. ES014-1, ES014-2, ES014-3, ES014-4, ES014-5, ES014-6, ES014-7) bound to MOLP-8 cells in a dose-dependent manner. As shown in FIG. 27B, ES014-1 bound to CHOK1/hCD39 cells in a dose-dependent manner. The binding features of the other tested anti-CD39/TGFβ Trap molecules (e.g. ES014-2, ES014-3, ES014-4, ES014-5, ES014-6, ES014-7) with CHOK1/hCD39 cells were similar and not shown herein.

### Example 11. Simultaneous Binding of Anti-CD39/TGFβ Trap Molecules to CD39 and TGFβ1

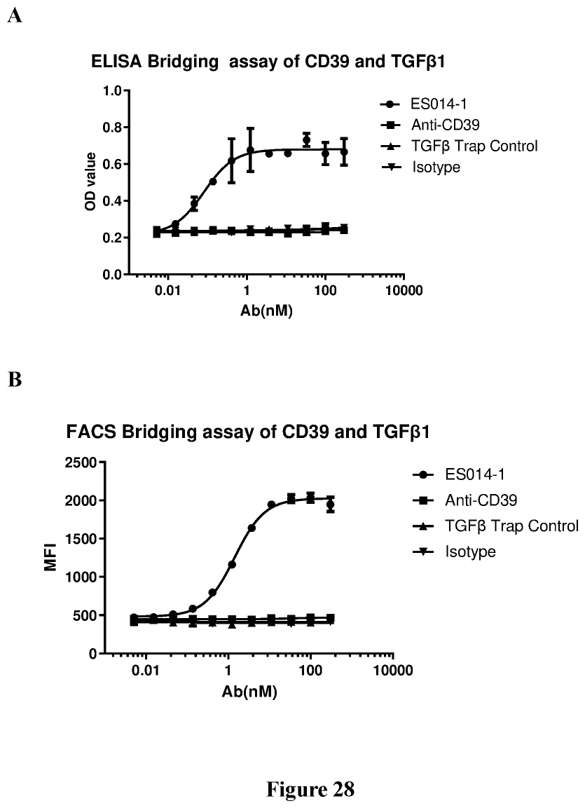

CD39/His protein (Sino Biological)—coated plates or CHO-K1/hCD39 cells were incubated with serial dilutions of ES014-1, anti-CD39 Ab or TGFβ trap control, followed by biotinylated TGFβ1. Binding was evaluated using streptavidin-horseradish peroxidase or streptavidin-fluorescein isothiocyanate. Optical densities (OD) were read at 450 nm. Data are means±SD, and nonlinear best fits are shown (n=2 technical replicates). The results were shown in FIG. 28. As shown in FIG. 28, the representative anti-CD39/TGFβ Trap molecule (ES014-1) could simultaneously bind to CD39 and TGFβ by ELISA detection (FIG. 28A) and FACS detection (FIG. 28B), respectively. The results of the other tested anti-CD39/TGFβ Trap molecules (e.g. ES014-2, ES014-3, ES014-4, ES014-5, ES014-6, ES014-7) were similar and not shown herein.

### Example 12. Anti-CD39/TGFβ Trap Molecules Inhibit TGFβ Signal

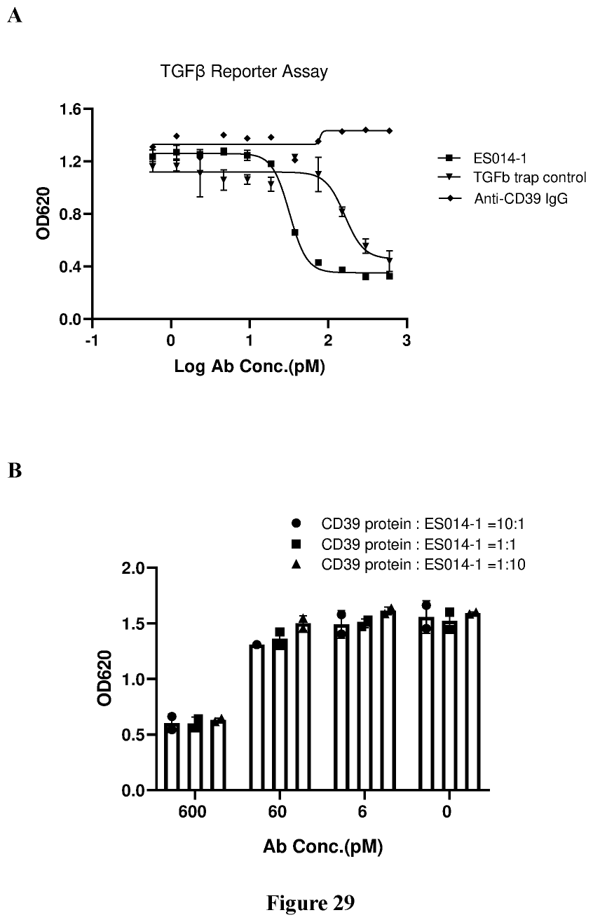

HEK-Blue™ TGF-β reporter cells assay (InvivoGen) was used to evaluate the effect of anti-CD39/TGFβ Trap molecules on canonical TGFβ signaling. Serial dilutions of anti-CD39/TGFβ Trap molecules or anti-CD39 were incubated with HEK-Blue™ TGF-β reporter cells for 24 hours in the presence of recombinant human TGF-β1 (5 ng/ml). Anti-CD39/TGFβ Trap molecules, but not anti-CD39, blocked TGF-β canonical signaling [half-maximal inhibitory concentration (IC 50)=32 μM] in a TGF-β SMAD reporter assay in transfected HEK293 cells (FIG. 29A). Besides, pre-incubate the anti-CD39/TGFβ Trap molecules with CD39 protein didn't affect the TGF-β neutralizing activity of the anti-CD39/TGFβ Trap molecules (FIG. 29B). FIGS. 29A and 29B show the results of the representative anti-CD39/TGFβ Trap molecule ES014-1. The results of the other tested anti-CD39/TGFβ Trap molecules (e.g. ES014-2, ES014-3, ES014-4, ES014-5, ES014-6, ES014-7) were similar and not shown herein.

### Example 13. Effect of the Anti-CD39/TGFβ Trap Molecules on CD39 Activity on Malignant Cells

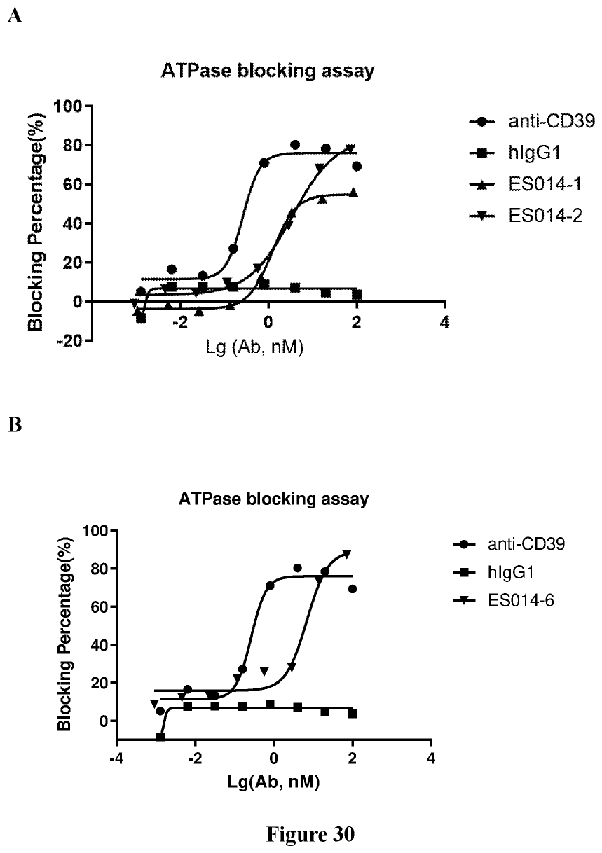

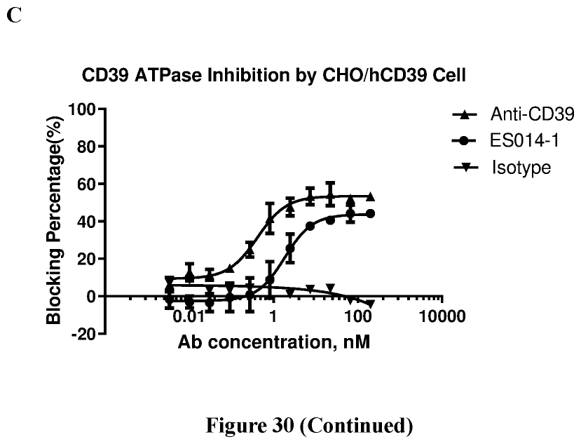

The ability of anti-CD39/TGFβ Trap molecules to inhibit the enzymatic activity of CD39 on malignant cell lines was measured using a cell-titer glo (CTG) assay. Briefly, cells were treated for 60 min with anti-CD39/TGFβ Trap molecules, anti-CD39 antibody or control antibody and 100 μM ATP. The remaining ATP level was measured using a CellTiterGlo Luminescent assay kit (Promega). MOLP-8 (human multiple myeloma cell line) or CHO/hCD39 cells were used in this assay. As shown in FIGS. 30A-B, treatment of MOLP-8 cell with a range of concentrations of anti-CD39/TGFβ Trap molecules or a control antibody, as indicated, in the presence of ATP resulted in a dose-dependent inhibition of CD39 activity by tested molecules ES014-1, ES014-2 and ES014-6. Inhibition of CD39 activity was determined by the extent of ATP remaining and expressed as % inhibition. As the IC 50 listed in Table 28 below, ES014-1, ES014-2 and ES014-6 showed strong inhibitory activity with nanomolar IC₅₀. As shown in FIG. 30C, treatment of CHO/hCD39 cells with a range of concentrations of an exemplary anti-CD39/TGFβ Trap molecule ES014-1 or a control antibody, as indicated, in the presence of ATP resulted in a dose-dependent inhibition of CD39 activity by all antibodies tested. The treatment results of CHO/hCD39 cells with a range of concentrations of the other tested anti-CD39/TGFβ Trap molecules (e.g. ES014-2, ES014-3, ES014-4, ES014-5, ES014-6, ES014-7) were similar and not shown herein.

TABLE 28
IC₅₀ of anti-CD39/TGFβ Trap molecules
to CD39 ATPase activity on MOLP-8 cells

| Anti-CD39/TGFβ Trap | ES014-1 | ES014-2 | ES014-6 | CD39 |
|---|---|---|---|---|
| IC₅₀ (nM) | 1.21 | 3.42 | 6.9 | 0.2641 |

### Example 14. Anti-CD39/TGFβ Trap Molecule Binding Affinities

A representative anti-CD39/TGFβ Trap molecule ES014-1 was characterized for binding affinity against human TGFβ1 or CD39 using Octet assay (ForeBio) according to manufacturer's manual, separately. Briefly, the antibodies were coupled on sensors and then the sensors were dipped into TGFβ or CD39 protein gradients (start at 200 nM, with 2-fold dilution and totally 8 doses). Their binding responses were measured in real-time and results were fit globally. The affinity data of the tested molecule ES014-1 are summarized in Table 29 below. The affinity data of the other tested molecules (e.g. ES014-2, ES014-3, ES014-4, ES014-5, ES014-6, ES014-7) were similar and not shown herein.

TABLE 29
Binding affinity of bispecific antibody ES014-1 to
human TGFβ1 and CD39 as measured by Octet assay

| Binding Target | K_{D} (M) | kon (1/Ms) | kdis (1/s) |
|---|---|---|---|
| TGFβ1 | 3.70E−11 | 6.32E+06 | 2.34E−04 |
| CD39 | 3.76E−10 | 9.14E+04 | 3.44E−05 |

### Example 15. Treg Suppression Assay

As Treg is a major secretion source of TGFβ, and CD39 expresses on Treg and DCs, the relative ability of anti-CD39/TGFβ Trap molecules to counteract Treg-mediated suppression of T cells was examined using Treg suppression assay. Briefly, CD3⁺ total T cells isolated from human PBMC were added to allogeneic DCs that had been pulsed with IL-4 and GM-CSF in the presence of autologous CD4⁺/CD25⁺ naturally Tregs (nTregs) isolated from PBMC and expanded in X-vivo medium in presence of IL2, anti-CD3/CD28 and Rapamycin with a ratio of 1:1:10.

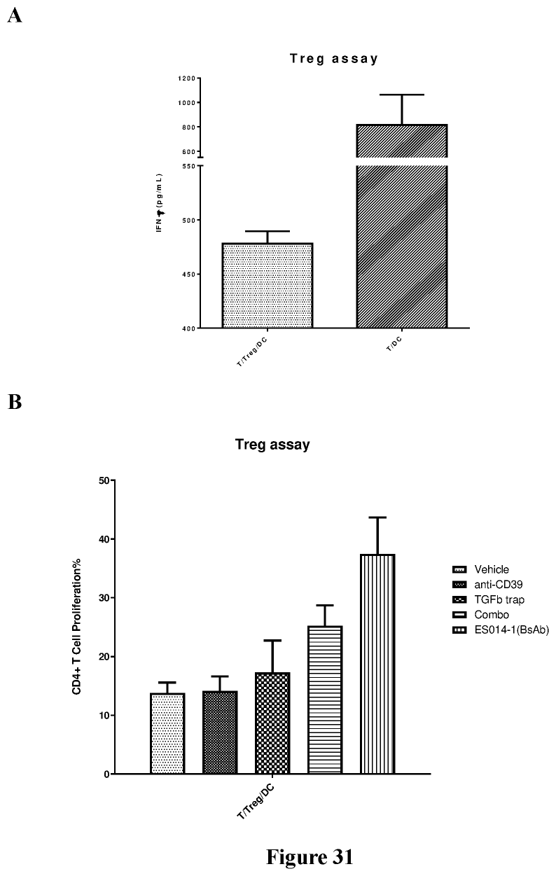

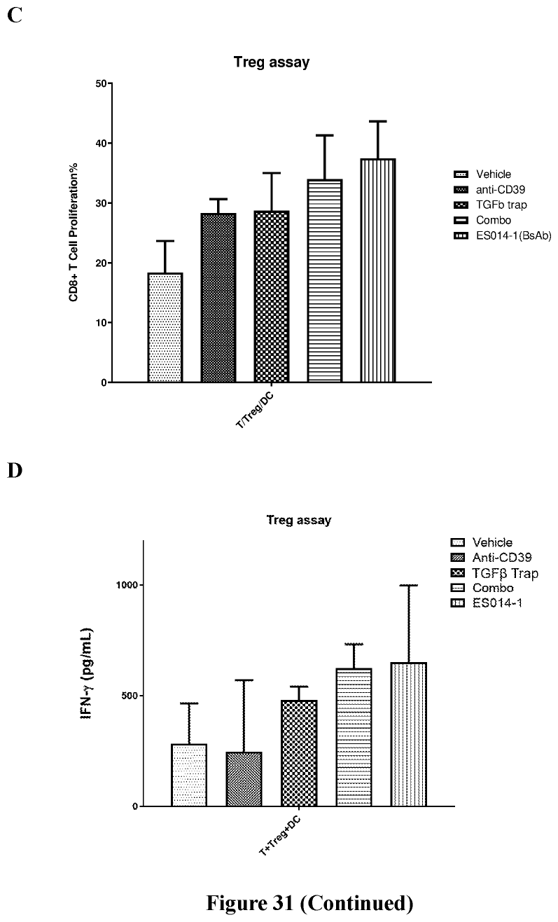

Following culture of these mixed lymphocytes for 3 days with either anti-CD39/TGFβ Trap molecule, anti-CD39 antibody, soluble TGFβ trap or combination of anti-CD39 antibody with TGFβ trap, T cell's function were evaluated through measuring CD4⁺ and CD8⁺ T cell proliferation with CFSE cell tracer and IFNγ secretion by HTRF(Cisbo). As expected, the addition of autologous Tregs suppressed the activation of T cells triggered by allogeneic DCs (FIG. 31A). a representative anti-CD39/TGFβ Trap molecule ES014-1 was more effective than anti-CD39 antibody, soluble TGFβ trap or combination thereof in counteracting Treg-mediated suppression and restoring activation of T cells in the presence of autologous Tregs (FIGS. 31B-D). These data demonstrate that anti-CD39/TGFβ Trap molecule ES014-1 is more effective than anti-CD39 antibody, soluble TGFβ trap or combination thereof in the recovery of T cell function. The other tested molecules (e.g. ES014-2, ES014-3, ES014-4, ES014-5, ES014-6, ES014-7) also showed similar effect (data not shown).

### Example 16. Anti-CD39/TGFβ Trap Molecules Inhibit Human T Cell Apoptosis without Stimulation

2×10⁴ purified total CD3⁺ T cells from human PBMC were cultured overnight and incubated with anti-CD39/TGFβ Trap molecules ES014-1 and ES014-2, TGFβR dead mutant ES014_v2, anti-CD39 dead mutant ES014_v1, and double negative mutant ES014_v3 overnight in an equal molar concentration. Apoptosis of human T cells was measured by APC-labeled Annexin V and PI by flow cytometry according to manufacturer's instructions.

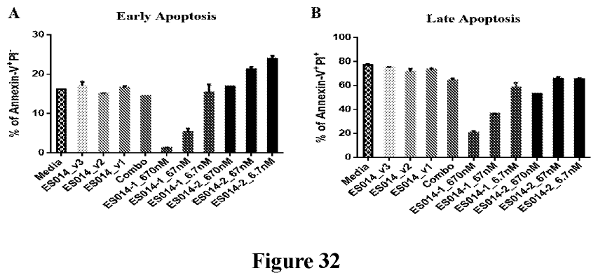

As shown in FIG. 32A and FIG. 32B, anti-CD39/TGFβ Trap molecules ES014-1 and ES014-2 inhibited human T cell apoptosis in a dose-dependent way compared to TGFβR dead mutant ES014_v2, anti-CD39 dead mutant ES014_v1, and double negative mutant ES014_v3.

### Example 17. Anti-CD39/TGFβ Trap Molecules Promote Human T Cell Survival and Activation Over Stimulation

5×10³ purified total CD3⁺ T cells were cocultured with the same molar of anti-CD39/TGFβ Trap molecules ES014-1 and ES014-2, anti-CD39 antibody ES014_v2, TGF-beta trap ES014_v1, combo (ES014_v2 and ES014_v1) and double mutant antibody ES014_v3 as control for 4 days in the presence of anti-CD3 and anti-CD28 beads stimulation. T cell function were quantified by measuring T survival with live-dead stained, T cell proliferation with celltrace labeling, T activation with CD25 expression and cytokine production.

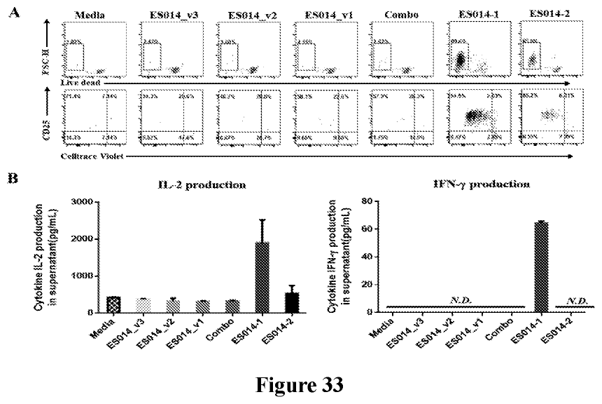

As shown in FIG. 33A, there were most of dead cells with anti-CD3/CD28 stimulation in all the groups except anti-CD39/TGFβ Trap molecules group. Moreover, the live cells in anti-CD39/TGFβ Trap molecules group maintained higher cell proliferation and activation (FIG. 33A), and IL-2 and IFN-γ production (FIG. 33B). These data demonstrate that anti-CD39/TGFβ Trap molecules are more effective than anti-CD39 antibody, TGFβ trap or combination thereof in T cell over-activation.

### Example 18. Anti-CD39/TGFβ Trap Molecules Block TGF-Beta Induced Foxp3 Expression on Total T Cells

5×10⁴ purified T cells were pretreated with the same molar of anti-CD39/TGFβ Trap molecules (ES014-1 and ES014-2), anti-CD39 antibody (ES014_v2), TGF-beta trap (ES014_v1), combo (ES014_v2+ES014_v1) and control antibody (ES014_v3) for 30 min in the presence of anti-CD3 and anti-CD28 beads stimulation and added 10 ng/ml TGF-beta. Treg differentiation were measured after treated with TGF-beta for 4 days.

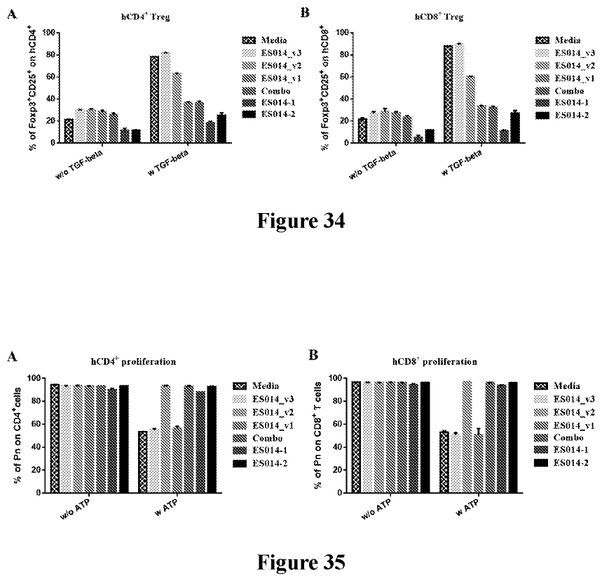

As shown in FIGS. 34A and 34B, treatment with anti-CD39/TGFβ Trap molecules (ES014-1 and ES014-2), TGF-beta trap (ES014_v1) and combo (ES014_v2+ES014_v1) could block TGF-beta induced Foxp3 expression on CD4+ and CD8⁺ T cells compared with those treated with media, Anti-CD39 (ES014_v2) and control antibody (ES014_v3). Notably, the blocking effect in treated anti-CD39/TGFβ Trap molecules group showed better activity than those in treated TGF-beta trap (ES014_v1) and combo (ES014_v2+ES014_v1) groups, especially anti-CD39/TGFβ Trap molecule ES014-1.

### Example 19. Anti-CD39/TGFβ Trap Molecules Restore ATP Induced Inhibition on Human T Cell Proliferation

5×10⁴ purified T cells were labeled with celltrace violet and pretreated with anti-CD39/TGFβ Trap molecules (ES014-1 and ES014-2), anti-CD39 antibody (ES014_v2), TGF-beta trap (ES014_v1), combo (ES014_v2+ES014_v1) and control antibody (ES014_v3) overnight in the presence of anti-CD3 and anti-CD28 beads stimulation. On day 1, 200 μM ATP were added and the T proliferation were measured after treated with ATP for 3 days. As shown in FIGS. 35A and 35B, treatment with anti-CD39/TGFβ Trap molecules (ES014-1 and ES014-2), anti-CD39 antibody (ES014_v2) and combo (ES014_v2+ES014_v1) could reverse ATP induced inhibition on CD4+ and CD8+T proliferation compared with those treated with media, TGF-beta trap (ES014_v1) and control antibody (ES014_v3). The restored T cell proliferation result showed anti-CD39/TGFβ Trap molecules' effect on blocking CD39 activity, which was consistent with the ATPase inhibitory activity results.

What is claimed is:

1. A conjugate molecule comprising a CD39 inhibitory portion capable of interfering interaction between CD39 and its substrate, and a TGFβ inhibitory portion capable of interfering interaction between TGFβ and its receptor.

2. The conjugate molecule of claim 1, wherein the CD39 inhibitory portion is capable of interfering interaction between CD39 and ATP/ADP, and/or the TGFβ inhibitory portion is capable of interfering interaction between TGFβ and TGFβ receptor.

3. The conjugate molecule of claim 1 or 2, wherein the CD39 inhibitory portion is an antagonist of CD39 selected from a group consisting of a CD39-binding agent, an RNAi that targets an encoding sequence of CD39, an antisense nucleotide that targets an encoding sequence of CD39, and an agent that competes with CD39 to bind to its substrate.

4. The conjugate molecule of any one of claims 1-3, wherein the TGFβ inhibitory portion is an antagonist of TGFβ selected from a group consisting of a TGFβ-binding agent, an RNAi that targets an encoding sequence of TGFβ, an antisense nucleotide that targets an encoding sequence of TGFβ, and an agent that competes with TGFβ to bind to its receptor.

5. The conjugate molecule of any one of claims 1-4, wherein the CD39-binding agent is selected from the group consisting of an antibody or an antigen-binding fragment thereof that specifically recognizes CD39, and a small molecule compound that binds to CD39; and/or the TGFβ-binding agent is selected from the group consisting of an antibody or an antigen-binding fragment thereof that specifically recognizes TGFβ, and a small molecule compound that binds to TGFβ.

6. The conjugate molecule of any one of claims 1-5, wherein the conjugate molecule is a fusion protein comprising a CD39-binding domain linked to a TGFβ-binding domain.

7. The conjugate molecule of claim 6, wherein the TGFβ-binding domain binds to human and/or mouse TGFβ.

8. The conjugate molecule of claim 6, wherein the TGFβ-binding domain binds to human TGFβ1, human TGFβ2, and/or human TGFβ3.

9. The conjugate molecule of any one of claims 6-8, wherein the TGFβ-binding domain comprises an extracellular domain (ECD) of a TGFβ receptor.

10. The conjugate molecule of claim 9, wherein the TGFβ receptor is TGFβ Receptor I (TGFβRI), TGFβ Receptor II (TGFβRII), or TGFβ Receptor III (TGFβRIII).

11. The conjugate molecule of claim 9 or 10, wherein the ECD comprises an amino acid sequence of SEQ ID NO: 163, SEQ ID NO: 164, SEQ ID NO: 165, or an amino acid sequence having at least 85% sequence identity thereof yet retaining binding specificity to TGFβ.

12. The conjugate molecule of any one of claims 6-11, wherein the TGFβ-binding domain comprises two or more ECDs of a TGFβ receptor.

13. The conjugate molecule of claim 12, wherein the two or more ECDs are derived from the same TGFβ receptor, or are derived from at least two different TGFβ receptors.

14. The conjugate molecule of claim 12, wherein the two or more ECDs comprise a first ECD derived from TGFβRI and a second ECD derived from TGFβRII.

15. The conjugate molecule of any one of claims 12-14, wherein the two or more ECDs are operably linked in series.

16. The conjugate molecule of claim 15, wherein the two or more ECDs are linked via a first linker.

17. The conjugate molecule of claim 16, wherein the TGFβ-binding domain comprises an amino acid sequence selected from the group consisting of SEQ ID NO: 166, SEQ ID NO: 167, SEQ ID NO: 168, SEQ ID NO: 169, SEQ ID NO: 170, SEQ ID NO: 171, or any combination thereof.

18. The conjugate molecule of any one of claims 6-17, wherein the CD39-binding domain binds to human CD39.

19. The conjugate molecule of any one of claims 6-18, wherein the TGFβ-binding domain is linked to the CD39 binding domain via a second linker.

20. The conjugate molecule of any one of claims 6-19, wherein the CD39-binding domain comprises an anti-CD39 antibody moiety.

21. The conjugate molecule of claim 20, wherein the anti-CD39 antibody moiety comprises a heavy chain variable region and a light chain variable region.

22. The conjugate molecule of claim 21, wherein the anti-CD39 antibody moiety further comprises a heavy chain constant domain appended to a carboxyl terminus of the heavy chain variable region.

23. The conjugate molecule of claim 21 or 22, wherein the anti-CD39 antibody moiety further comprises a light chain constant domain appended to a carboxyl terminus of the light chain variable region.

24. The conjugate molecule of any one of claims 21-23, wherein the TGFβ-binding domain is linked to the anti-CD39 antibody moiety at a position selected from the group consisting of: 1) amino terminus of the heavy chain variable region, 2) amino terminus of the light chain variable region, 3) carboxyl terminus of the heavy chain variable region; 4) carboxyl terminus of the light chain variable region; 5) carboxyl terminus of the heavy chain constant region; and 6) carboxyl terminus of the light chain constant region, of the anti-CD39 antibody moiety.

25. The conjugate molecule of any one of claims 21-24, wherein the fusion protein comprises two or more TGFβ-binding domains which are (i) all linked to the heavy chain variable region of the anti-CD39 antibody moiety, or (ii) are all linked to the light chain variable region of the anti-CD39 antibody moiety.

26. The conjugate molecule of any one of claims 21-24, wherein the fusion protein comprises two or more TGFβ-binding domains which are linked to the heavy and the light chain variable region of the anti-CD39 antibody moiety, respectively.

27. The conjugate molecule of any one of claims 21-24, wherein the fusion protein comprises two or more TGFβ-binding domains which are all linked to the heavy chain constant region of the anti-CD39 antibody moiety.

28. The conjugate molecule of any one of claims 21-24, wherein the fusion protein comprises two or more TGFβ-binding domains which are all linked to the light chain constant region of anti-CD39 antibody moiety.

29. The conjugate molecule of any one of claims 21-24, wherein the fusion protein comprises two or more TGFβ-binding domains which are linked to the heavy and the light chain constant regions of the anti-CD39 antibody moiety, respectively.

30. The conjugate molecule of any one of claims 6-29, wherein the fusion protein comprises two, three, four, five, six or more TGFβ-binding domains.

31. The conjugate molecule of any one of claims 16-30, wherein the first and/or the second linker is selected from the group consisting of a cleavable linker, a non-cleavable linker, a peptide linker, a flexible linker, a rigid linker, a helical linker, and a non-helical linker.

32. The conjugate molecule of claim 31, wherein the first and/or the second linker comprises a peptide linker.

33. The conjugate molecule of claim 32, wherein the peptide linker comprises a GS linker.

34. The conjugate molecule of claim 33, wherein the GS linker comprises one or more repeats of SEQ ID NO: 177 (GGGS) or SEQ ID NO: 173 (GGGGS), or comprises an amino acid sequence of SEQ ID NO: 182 (GGGGSGGGGSGGGGSG).

35. The conjugate molecule of any one of claims 20-34, wherein the anti-CD39 antibody moiety comprises a heavy chain variable region comprising HCDR1, HCDR2 and HCDR3 and/or a light chain variable region comprising LCDR1, LCDR2 and LCDR3, wherein
a) the HCDR1 comprises an amino acid sequence selected from the group consisting of NYGMN (SEQ ID NO: 1), KYWMN (SEQ ID NO: 2), NYWMN (SEQ ID NO: 3), DTFLH (SEQ ID NO: 4), DYNMY (SEQ ID NO: 5), and DTYVH (SEQ ID NO: 6); and
b) the HCDR2 comprises an amino acid sequence selected from the group consisting of LINTYTGEPTYADDFKD (SEQ ID NO: 7), EIRLKSNKYGTHYAESVKG (SEQ ID NO: 8), QIRLNPDNYATHX₁AESVKG (SEQ ID NO: 9), X₅₈IDPAX₅₉X₆₀NIKYDPKFQG (SEQ ID NO: 151), FIDPYNGYTSYNQKFKG (SEQ ID NO: 11), and RIDPAIDNSKYDPKFQG (SEQ ID NO: 12); and
c) the HCDR3 comprises an amino acid sequence selected from the group consisting of KGIYYDYVWFFDV (SEQ ID NO: 13), QLDLYWFFDV (SEQ ID NO: 14), HGX₂RGFAY (SEQ ID NO: 15), SPYYYGSGYRIFDV (SEQ ID NO: 16), IYGYDDAYYFDY (SEQ ID NO: 17), and YYCALYDGYNVYAMDY (SEQ ID NO: 18); and
d) the LCDR1 comprises an amino acid sequence selected from the group consisting of KASQDINRYIA (SEQ ID NO: 19), RASQSISDYLH (SEQ ID NO: 20), KSSQSLLDSDGRTHLN (SEQ ID NO: 21), SAFSSVNYMH (SEQ ID NO: 22), SATSSVSYMH (SEQ ID NO: 23), and RSSKNLLHSNGITYLY (SEQ ID NO: 24); and
e) the LCDR2 comprises an amino acid sequence selected from the group consisting of YTSTLLP (SEQ ID NO: 25), YASQSIS (SEQ ID NO: 26), LVSKLDS (SEQ ID NO: 27), TTSNLAS (SEQ ID NO: 28), STSNLAS (SEQ ID NO: 29), and RASTLAS (SEQ ID NO: 30); and
f) the LCDR3 comprises an amino acid sequence selected from the group consisting of LQYSNLLT (SEQ ID NO: 31), QNGHSLPLT (SEQ ID NO: 32, WQGTLFPWT (SEQ ID NO: 33), QQRSTYPFT (SEQ ID NO: 34), QQRITYPFT (SEQ ID NO: 35), and AQLLELPHT (SEQ ID NO: 36);
wherein X₁ is Y or F, X₂ is S or T, X₅₈ is R or K, X₅₉ is N, G, S or Q, X₆₀ is G, A or D.

36. The conjugate molecule of claim 35, wherein:
a) the HCDR1 comprises the amino acid sequence of SEQ ID NO: 3, and/or
b) the HCDR2 comprises the amino acid sequence of SEQ ID NO: 9, and/or
c) the HCDR3 comprises the amino acid sequence of SEQ ID NO: 15, and/or
d) the LCDR1 comprises the amino acid sequence of SEQ ID NO: 21, and/or
e) the LCDR2 comprises the amino acid sequence of SEQ ID NO: 27, and/or
f) the LCDR3 comprises the amino acid sequence of SEQ ID NO: 33,
wherein X₁ and X₂ are as defined in claim 35.

37. The conjugate molecule of claim 36, wherein:
the HCDR2 comprises an amino acid sequence selected from the group consisting of SEQ ID NO: 37 and SEQ ID NO: 38, and/or
the HCDR3 comprises an amino acid sequence selected from the group consisting of SEQ ID NO: 40 and SEQ ID NO: 41.

38. The conjugate molecule of claim 35, wherein:
a) the HCDR1 comprises the amino acid sequence of SEQ ID NO: 4, and/or
b) the HCDR2 comprises the amino acid sequence of SEQ ID NO: 151, and/or
c) the HCDR3 comprises the amino acid sequence of SEQ ID NO: 16, and/or
d) the LCDR1 comprises the amino acid sequence of SEQ ID NO: 22, and/or
e) the LCDR2 comprises the amino acid sequence of SEQ ID NO: 28, and/or
f) the LCDR3 comprises the amino acid sequence of SEQ ID NO: 34,
wherein X₅₈, X₅₉ and X₆₀ are as defined in claim 35.

39. The conjugate molecule of any one of claims 35-38, wherein the heavy chain variable region comprises:
a) a HCDR1 comprising the sequence of SEQ ID NO: 1, a HCDR2 comprising the sequence of SEQ ID NO: 7, and a HCDR3 comprising the sequence of SEQ ID NO: 13; or
b) a HCDR1 comprising the sequence of SEQ ID NO: 2, a HCDR2 comprising the sequence of SEQ ID NO: 8, and a HCDR3 comprising the sequence of SEQ ID NO: 14; or
c) a HCDR1 comprising the sequence of SEQ ID NO: 3, a HCDR2 comprising the sequence of SEQ ID NO: 37, and a HCDR3 comprising the sequence of SEQ ID NO: 40; or
d) a HCDR1 comprising the sequence of SEQ ID NO: 3, a HCDR2 comprising the sequence of SEQ ID NO: 38, and a HCDR3 comprising the sequence of SEQ ID NO: 41; or
e) a HCDR1 comprising the sequence of SEQ ID NO: 4, a HCDR2 comprising the sequence of SEQ ID NO: 10, and a HCDR3 comprising the sequence of SEQ ID NO: 16; or
f) a HCDR1 comprising the sequence of SEQ ID NO: 4, a HCDR2 comprising a sequence selected from the group consisting of SEQ ID NOs: 134, 135, 136, 137, 138, and 139, and a HCDR3 comprising the sequence of SEQ ID NO: 16; or
g) a HCDR1 comprising the sequence of SEQ ID NO: 5, a HCDR2 comprising the sequence of SEQ ID NO: 11, and a HCDR3 comprising the sequence of SEQ ID NO: 17; or
h) a HCDR1 comprising the sequence of SEQ ID NO: 6, a HCDR2 comprising the sequence of SEQ ID NO: 12, and a HCDR3 comprising the sequence of SEQ ID NO: 18.

40. The conjugate molecule of any one of claims 35-39, wherein the light chain variable region comprises:
a) a LCDR1 comprising the sequence of SEQ ID NO: 19, a LCDR2 comprising the sequence of SEQ ID NO: 25, and a LCDR3 comprising the sequence of SEQ ID NO: 31; or
b) a LCDR1 comprising the sequence of SEQ ID NO: 20, a LCDR2 comprising the sequence of SEQ ID NO: 26, and a LCDR3 comprising the sequence of SEQ ID NO: 32; or
c) a LCDR1 comprising the sequence of SEQ ID NO: 21, a LCDR2 comprising the sequence of SEQ ID NO: 27, and a LCDR3 comprising the sequence of SEQ ID NO: 33; or
d) a LCDR1 comprising the sequence of SEQ ID NO: 22, a LCDR2 comprising the sequence of SEQ ID NO: 28, and a LCDR3 comprising the sequence of SEQ ID NO: 34; or
e) a LCDR1 comprising the sequence of SEQ ID NO: 23, a LCDR2 comprising the sequence of SEQ ID NO: 29, and a LCDR3 comprising the sequence of SEQ ID NO: 35; or
f) a LCDR1 comprising the sequence of SEQ ID NO: 24, a LCDR2 comprising the sequence of SEQ ID NO: 30, and a LCDR3 comprising the sequence of SEQ ID NO: 36.

41. The conjugate molecule of any one of claims 35-40, wherein:
a) the HCDR1 comprises the sequence of SEQ ID NO: 1, the HCDR2 comprises the sequence of SEQ ID NO: 7, the HCDR3 comprises the sequence of SEQ ID NO: 13; the LCDR1 comprises the sequence of SEQ ID NO: 19, the LCDR2 comprises the sequence of SEQ ID NO: 25, and the LCDR3 comprises the sequence of SEQ ID NO: 31; or
b) the HCDR1 comprises the sequence of SEQ ID NO: 2, the HCDR2 comprises the sequence of SEQ ID NO: 8, the HCDR3 comprises the sequence of SEQ ID NO: 14; the LCDR1 comprises the sequence of SEQ ID NO: 20, the LCDR2 comprises the sequence of SEQ ID NO: 26, and the LCDR3 comprises the sequence of SEQ ID NO: 32; or
c) the HCDR1 comprises the sequence of SEQ ID NO: 3, the HCDR2 comprises the sequence of SEQ ID NO: 37, the HCDR3 comprises the sequence of SEQ ID NO: 40; the LCDR1 comprises the sequence of SEQ ID NO: 21, the LCDR2 comprises the sequence of SEQ ID NO: 27, and the LCDR3 comprises the sequence of SEQ ID NO: 33; or
d) the HCDR1 comprises the sequence of SEQ ID NO: 3, the HCDR2 comprises the sequence of SEQ ID NO: 38, the HCDR3 comprises the sequence of SEQ ID NO: 41; the LCDR1 comprises the sequence of SEQ ID NO: 21, the LCDR2 comprises the sequence of SEQ ID NO: 27, and the LCDR3 comprises the sequence of SEQ ID NO: 33; or
e) the HCDR1 comprises the sequence of SEQ ID NO: 4, the HCDR2 comprises the sequence of SEQ ID NO: 10, the HCDR3 comprises the sequence of SEQ ID NO: 16; the LCDR1 comprises the sequence of SEQ ID NO: 22, the LCDR2 comprises the sequence of SEQ ID NO: 28, and the LCDR3 comprises the sequence of SEQ ID NO: 34; or
f) the HCDR1 comprises the sequence of SEQ ID NO: 4, the HCDR2 comprises a sequence selected from the group consisting of SEQ ID NOs: 134, 135, 136, 137, 138, and 139, the HCDR3 comprises the sequence of SEQ ID NO: 16; the LCDR1 comprises the sequence of SEQ ID NO: 22, the LCDR2 comprises the sequence of SEQ ID NO: 28, and the LCDR3 comprises the sequence of SEQ ID NO: 34; or
g) the HCDR1 comprises the sequence of SEQ ID NO: 5, the HCDR2 comprises the sequence of SEQ ID NO: 11, the HCDR3 comprises the sequence of SEQ ID NO: 17; the LCDR1 comprises the sequence of SEQ ID NO: 23, the LCDR2 comprises the sequence of SEQ ID NO: 29, and the LCDR3 comprises the sequence of SEQ ID NO: 35; or
h) the HCDR1 comprises the sequence of SEQ ID NO: 6, the HCDR2 comprises the sequence of SEQ ID NO: 12, the HCDR3 comprises the sequence of SEQ ID NO: 18; the LCDR1 comprises the sequence of SEQ ID NO: 24, the LCDR2 comprises the sequence of SEQ ID NO: 30, and the LCDR3 comprises the sequence of SEQ ID NO: 36.

42. The conjugate molecule of any one of claims 35-41, wherein the anti-CD39 antibody moiety further comprises one or more of heavy chain framework region HFR1, HFR2, HFR3 and HFR4, and/or one or more of light chain framework region LFR1, LFR2, LFR3 and LFR4, wherein:
the HFR1 comprises a sequence selected from the group consisting of SEQ ID NOs: 84-86, 115, 119-120, and 131;
the HFR2 comprises a sequence selected from the group consisting of SEQ ID NOs: 87-90, and 121-123;
the HFR3 comprises a sequence selected from the group consisting of SEQ ID NOs: 91-97, 116-117, and 124-125;
the HFR4 comprises a sequence selected from the group consisting of SEQ ID NOs: 79 and 118;
the LFR1 comprises a sequence selected from the group consisting of SEQ ID NOs: 98-103 and 127-129;
the LFR2 comprises a sequence selected from the group consisting of SEQ ID NOs: 104, 105 and 130;
the LFR3 comprises a sequence selected from the group consisting of SEQ ID NOs: 106-110 and 132-133, and
the LFR4 comprises a sequence selected from the group consisting of SEQ ID NOs: 83 and 153.

43. The conjugate molecule of any one of claims 35-42, wherein the anti-CD39 antibody moiety comprises a heavy chain variable region comprising an amino acid sequence selected from the group consisting of SEQ ID NOs: 60, 62, 64, 66, 140, 141, 142, 146, 147, and 39, or an amino acid sequence having at least 80% sequence identity thereof yet retaining specific binding specificity to human CD39, and
a light chain variable region comprising an amino acid sequence selected from the group consisting of SEQ ID NOs: 61, 63, 65, 67, 143, 144, 145, 111, 112, and 63, or an amino acid sequence having at least 80% sequence identity thereof yet retaining specific binding specificity to human CD39.

44. The conjugate molecule of any one of claims 35-43, wherein the anti-CD39 antibody moiety comprises a heavy chain variable region comprising an amino acid sequence selected from the group consisting of SEQ ID NOs: 68, 70, 72, and 74, or an amino acid sequence having at least 80% sequence identity thereof yet retaining specific binding specificity to human CD39, and
a light chain variable region comprising an amino acid sequence selected from the group consisting of SEQ ID NOs: 69, 71, 73, and 75, or an amino acid sequence having at least 80% sequence identity thereof, yet retaining specific binding specificity to human CD39.

45. The conjugate molecule of any one of claims 35-44, wherein the anti-CD39 antibody moiety comprises:
a) a heavy chain variable region comprising the sequence of SEQ ID NO: 42 and a light chain variable region comprising the sequence of SEQ ID NO: 51; or
b) a heavy chain variable region comprising the sequence of SEQ ID NO: 43 and a light chain variable region comprising the sequence of SEQ ID NO: 52; or
c) a heavy chain variable region comprising the sequence of SEQ ID NO: 44 and a light chain variable region comprising the sequence of SEQ ID NO: 53; or
d) a heavy chain variable region comprising the sequence of SEQ ID NO: 45 and a light chain variable region comprising the sequence of SEQ ID NO: 54; or
e) a heavy chain variable region comprising the sequence of SEQ ID NO: 47 and a light chain variable region comprising the sequence of SEQ ID NO: 56; or
f) a heavy chain variable region comprising the sequence of SEQ ID NO: 49 and a light chain variable region comprising the sequence of SEQ ID NO: 58; or
g) a heavy chain variable region comprising the sequence of SEQ ID NO: 50 and a light chain variable region comprising the sequence of SEQ ID NO: 59, or
h) a heavy chain variable region comprising the sequence of SEQ ID NO: 60 and a light chain variable region comprising the sequence of SEQ ID NO: 63, or
i) a heavy chain variable region comprising the sequence of SEQ ID NO: 62 and a light chain variable region comprising the sequence of SEQ ID NO: 63, or
j) a heavy chain variable region comprising the sequence of SEQ ID NO: 64 and a light chain variable region comprising the sequence of SEQ ID NO: 63, or
k) a heavy chain variable region comprising the sequence of SEQ ID NO: 66 and a light chain variable region comprising the sequence of SEQ ID NO: 63, or
l) a heavy chain variable region comprising the sequence of SEQ ID NO: 60 and a light chain variable region comprising the sequence of SEQ ID NO: 65, or
m) a heavy chain variable region comprising the sequence of SEQ ID NO: 62 and a light chain variable region comprising the sequence of SEQ ID NO: 65, or
n) a heavy chain variable region comprising the sequence of SEQ ID NO: 64 and a light chain variable region comprising the sequence of SEQ ID NO: 65, or
o) a heavy chain variable region comprising the sequence of SEQ ID NO: 66 and a light chain variable region comprising the sequence of SEQ ID NO: 65, or
p) a heavy chain variable region comprising the sequence of SEQ ID NO: 60 and a light chain variable region comprising the sequence of SEQ ID NO: 67, or
q) a heavy chain variable region comprising the sequence of SEQ ID NO: 62 and a light chain variable region comprising the sequence of SEQ ID NO: 67, or
r) a heavy chain variable region comprising the sequence of SEQ ID NO: 64 and a light chain variable region comprising the sequence of SEQ ID NO: 67, or
s) a heavy chain variable region comprising the sequence of SEQ ID NO: 66 and a light chain variable region comprising the sequence of SEQ ID NO: 67, or
t) a heavy chain variable region comprising the sequence of SEQ ID NO: 140 and a light chain variable region comprising the sequence of SEQ ID NO: 61, or
u) a heavy chain variable region comprising the sequence of SEQ ID NO: 141 and a light chain variable region comprising the sequence of SEQ ID NO: 61, or
v) a heavy chain variable region comprising the sequence of SEQ ID NO: 142 and a light chain variable region comprising the sequence of SEQ ID NO: 61, or
w) a heavy chain variable region comprising the sequence of SEQ ID NO: 140 and a light chain variable region comprising the sequence of SEQ ID NO: 143, or
x) a heavy chain variable region comprising the sequence of SEQ ID NO: 141 and a light chain variable region comprising the sequence of SEQ ID NO: 143, or
y) a heavy chain variable region comprising the sequence of SEQ ID NO: 142 and a light chain variable region comprising the sequence of SEQ ID NO: 143, or
z) a heavy chain variable region comprising the sequence of SEQ ID NO: 140 and a light chain variable region comprising the sequence of SEQ ID NO: 144, or
aa) a heavy chain variable region comprising the sequence of SEQ ID NO: 141 and a light chain variable region comprising the sequence of SEQ ID NO: 144, or
bb) a heavy chain variable region comprising the sequence of SEQ ID NO: 142 and a light chain variable region comprising the sequence of SEQ ID NO: 144, or
cc) a heavy chain variable region comprising the sequence of SEQ ID NO: 140 and a light chain variable region comprising the sequence of SEQ ID NO: 145, or
dd) a heavy chain variable region comprising the sequence of SEQ ID NO: 141 and a light chain variable region comprising the sequence of SEQ ID NO: 145, or
ee) a heavy chain variable region comprising the sequence of SEQ ID NO: 142 and a light chain variable region comprising the sequence of SEQ ID NO: 145, or
ff) a heavy chain variable region comprising the sequence of SEQ ID NO: 146 and a light chain variable region comprising the sequence of SEQ ID NO: 111, or
gg) a heavy chain variable region comprising the sequence of SEQ ID NO: 146 and a light chain variable region comprising the sequence of SEQ ID NO: 112, or
hh) a heavy chain variable region comprising the sequence of SEQ ID NO: 147 and a light chain variable region comprising the sequence of SEQ ID NO: 111, or
ii) a heavy chain variable region comprising the sequence of SEQ ID NO: 39 and a light chain variable region comprising the sequence of SEQ ID NO: 63, or
jj) a heavy chain variable region comprising the sequence of SEQ ID NO: 68 and a light chain variable region comprising the sequence of SEQ ID NO: 69, or
kk) a heavy chain variable region comprising the sequence of SEQ ID NO: 70 and a light chain variable region comprising the sequence of SEQ ID NO: 69, or
ll) a heavy chain variable region comprising the sequence of SEQ ID NO: 72 and a light chain variable region comprising the sequence of SEQ ID NO: 69, or
mm) a heavy chain variable region comprising the sequence of SEQ ID NO: 74 and a light chain variable region comprising the sequence of SEQ ID NO: 69, or
nn) a heavy chain variable region comprising the sequence of SEQ ID NO: 68 and a light chain variable region comprising the sequence of SEQ ID NO: 71, or
oo) a heavy chain variable region comprising the sequence of SEQ ID NO: 70 and a light chain variable region comprising the sequence of SEQ ID NO: 71, or
pp) a heavy chain variable region comprising the sequence of SEQ ID NO: 72 and a light chain variable region comprising the sequence of SEQ ID NO: 71, or
qq) a heavy chain variable region comprising the sequence of SEQ ID NO: 74 and a light chain variable region comprising the sequence of SEQ ID NO: 71, or
rr) a heavy chain variable region comprising the sequence of SEQ ID NO: 68 and a light chain variable region comprising the sequence of SEQ ID NO: 73, or
ss) a heavy chain variable region comprising the sequence of SEQ ID NO: 70 and a light chain variable region comprising the sequence of SEQ ID NO: 73, or
tt) a heavy chain variable region comprising the sequence of SEQ ID NO: 72 and a light chain variable region comprising the sequence of SEQ ID NO: 73, or
uu) a heavy chain variable region comprising the sequence of SEQ ID NO: 74 and a light chain variable region comprising the sequence of SEQ ID NO: 73, or
vv) a heavy chain variable region comprising the sequence of SEQ ID NO: 68 and a light chain variable region comprising the sequence of SEQ ID NO: 75, or
ww) a heavy chain variable region comprising the sequence of SEQ ID NO: 70 and a light chain variable region comprising the sequence of SEQ ID NO: 75, or
xx) a heavy chain variable region comprising the sequence of SEQ ID NO: 72 and a light chain variable region comprising the sequence of SEQ ID NO: 75, or
yy) a heavy chain variable region comprising the sequence of SEQ ID NO: 74 and a light chain variable region comprising the sequence of SEQ ID NO: 75.

46. The conjugate molecule of any one of claims 35-45, wherein the anti-CD39 antibody moiety further comprises one or more amino acid residue substitutions or modifications yet retains specific binding specificity to human CD39.

47. The conjugate molecule of claim 46, wherein at least one of the substitutions or modifications is in one or more of the CDR sequences, and/or in one or more of the non-CDR sequences of the heavy chain variable region or light chain variable region.

48. The conjugate molecule of any one of claims 21-47, wherein the constant region is derived from human immunoglobulin (Ig), or optionally human IgG.

49. The conjugate molecule of claim 48, wherein the constant region is derived from human IgG1, IgG2, IgG3, IgG4, IgA1, IgA2 or IgM.

50. The conjugate molecule of any one of claims 35-49, wherein the anti-CD39 antibody moiety is humanized.

51. The conjugate molecule of any one of claims 19-50, wherein the anti-CD39 antibody moiety is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

52. The conjugate molecule of any one of preceding claims, wherein the conjugate molecule is capable of specifically binding to human CD39 at an EC₅₀ of no more than 10⁻⁸ M as measured by FACS assay.

53. The conjugate molecule of any one of preceding claims, wherein the conjugate molecule has one or more properties selected from the group consisting of:
a) specifically binding to human CD39 but not specifically binding to mouse CD39 as measured by FACS assay;
b) specifically binding to cynomolgus CD39 at an EC₅₀ of no more than 10⁻⁸ M as measured by FACS assay;
c) specifically binding to human CD39 at a K_{D} value of no more than 10⁻⁷ M (e.g. no more than 5×10⁻⁸ M, no more than 3×10⁻⁸ M, no more than 2×10⁻⁸ M, no more than 1×10⁻⁸ M, or no more than 8×10⁻⁹ M) as measured by Biacore assay;
d) specifically binding to human CD39 at a K_{D} value of no more than 10⁻⁸ M (e.g. no more than 8×10⁻⁹ M, no more than 5×10⁻⁹ M, no more than 4×10⁻⁹ M, no more than 3×10⁻⁹ M, no more than 1×10⁻⁹ M, or no more than 9×10⁻¹⁰ M) as measured by Octet assay;
e) inhibiting ATPase activity in a CD39 expressing cell at an IC 50 of no more than nM (e.g. no more than 1 nM, no more than 5 nM, no more than 10 nM, or no more than 30 nM) as measured by ATPase activity assay;
f) capable of enhancing ATP mediated monocytes activation at a concentration of no more than 10 nM (e.g. no more than 5 nM, no more than 3 nM, no more than 2 nM, no more than 1 nM, no more than 0.5 nM, or no more than 0.2 nM) as measured by analysis of CD80, CD86 and/or CD40 expression by FACS assay;
g) capable of enhancing ATP mediated T cell activation in PBMC at a concentration of no more than 25 nM as measured by IL-2 secretion, IFN-γ secretion, CD4+ or CD8+ T cells proliferation;
h) capable of enhancing ATP mediated dendritic cell (DC) activation at a concentration of no more than 25 nM (or no more than 10 nM, or no more than or no more than 1 nM, or no more than 0.5 nM) as measured by analysis of CD83 expression by FACS assay, or by the capability of the activated DC to promote T cell proliferation, or by the capability of the activated DC to promote IFN-γ production in the mix-lymphocyte reaction (MLR) assay;
i) capable of blocking the inhibition of CD4⁺ T cell proliferation induced by adenosine (hydrolyzed from ATP) at a concentration of no more than 1 nM (e.g. no more than 0.1 nM, no more than 0.01 nM) as measured by FACS assay;
j) capable of inhibiting tumor growth in a mammal a NK cell or macrophage cell dependent manner;
k) capable of reversing human CD8⁺ T cell proliferation which was inhibited by eATP as measured by T cell proliferation, CD25⁺ cells, and living cells population; and
l) capable of enhancing human macrophage IL1β release induced by LPS stimulation at a concentration of no more than 50 nM (or no more than 12.5 nM, or no more than 3.13 nM, or no more than 0.78 nM, or no more than 0.2 nM, or no more than 0.049 nM, or no more than 0.012 nM, or no more than 0.003 nM, or no more than 0.0008 nM) as measured by ELISA assay.

54. The conjugate molecule of any one of claims 1-34, wherein the CD39-binding domain competes for binding to human CD39 with an antibody comprising a heavy chain variable region comprising the sequence of SEQ ID NO: 43 and a light chain variable region comprising the sequence of SEQ ID NO: 52.

55. The conjugate molecule of any one of claims 1-34, wherein the CD39-binding domain competes for binding to human CD39 with an antibody comprising a heavy chain variable region comprising the sequence of SEQ ID NO: 44 and a light chain variable region comprising the sequence of SEQ ID NO: 53, or competes with an antibody comprising a heavy chain variable region comprising the sequence of SEQ ID NO: 45 and a light chain variable region comprising the sequence of SEQ ID NO: 54.

56. The conjugate molecule of any one of claims 1-34, wherein the CD39-binding domain competes for binding to human CD39 with an antibody comprising a heavy chain variable region comprising the sequence of SEQ ID NO: 47 and a light chain variable region comprising the sequence of SEQ ID NO: 56.

57. The conjugate molecule of any one of the preceding claims, wherein the CD39-binding domain specifically binds to an epitope of CD39, wherein the epitope comprises one or more residues selected from the group consisting of Q96, N99, E143, R147, R138, M139, E142, K5, E100, D107, V81, E82, R111, and V115.

58. The conjugate molecule of claim 57, wherein the epitope comprises one or more residues selected from the group consisting of Q96, N99, E143, and R147.

59. The conjugate molecule of claim 57, wherein the epitope comprises one or more residues selected from the group consisting of R138, M139, and E142.

60. The conjugate molecule of claim 57, wherein the epitope comprises one or more residues selected from the group consisting of K5, E100, and D107.

61. The conjugate molecule of claim 57, wherein the epitope comprises one or more residues selected from the group consisting of V81, E82, R111, and V115.

62. The conjugate molecule of any one of the preceding claims, wherein the CD39-binding domain specifically binds to a human CD39 comprising an amino acid sequence of SEQ ID NO: 162.

63. The conjugate molecule of any one of claims 56-62, wherein the CD39-binding domain is not derived from any of Antibody 9-8B, Antibody T895, and Antibody I394, wherein:
Antibody 9-8B comprises a heavy chain variable region comprising the sequence of SEQ ID NO: 46, and a light chain variable region comprising the sequence of SEQ ID NO: 48;
Antibody T895 comprises a heavy chain variable region comprising the sequence of SEQ ID NO: 55, and a light chain variable region comprising the sequence of SEQ ID NO: 57; and
Antibody I394 comprises a heavy chain variable region comprising the sequence of SEQ ID NO: 113, and a light chain variable region comprising the sequence of SEQ ID NO: 114.

64. The conjugate molecule of any one of the preceding claims, wherein the conjugate molecule has one or more properties selected from the group consisting of:
a) specifically binding to human TGFβ1, human TGFβ2, and/or human TGFβ3;
b) specifically binding to human TGFβ1 and mouse TGFβ1 with similar affinity;
c) specifically binding to human TGFβ1 at an EC₅₀ of no more than 3×10⁻¹¹ M (e.g. no more than 2×10⁻¹¹ M, no more than 1×10⁻¹¹ M, no more than 0.9×10⁻¹¹ M, no more than 0.8×10⁻¹¹ M, no more than 0.7×10⁻¹¹ M, no more than 0.6×10⁻¹¹ M, no more than 0.5×10⁻¹¹ M) as measured by ELISA assay;
d) capable of blocking human TGFβ1 and TGFβRII binding at an IC₅₀ of no more than 4×10⁻¹⁰ M (e.g. no more than 3×10⁻¹⁰ M, no more than 2×10⁻¹⁰ M, no more than 1×10⁻¹⁰ M, no more than 0.5×10⁻¹⁰ M) as measured by blocking assay;
e) capable of binding to human CD39 in a dose-dependent manner as measured by FACS assay;
f) capable of simultaneously binding to CD39 and TGFβ as measured by ELISA assay or FACS assay;
g) capable of inhibiting TGFβ signal at an IC₅₀ no more than 4×10⁻¹¹ M as measured by a TGF-β SMAD reporter assay;
h) capable of inhibiting ATPase activity in a CD39 expressing cell at an IC₅₀ of no more than 7×10⁻¹⁰ m (e.g. no more than 6×10⁻¹⁰ M, no more than 5×10⁻¹⁰ M, no more than 4×10⁻¹⁰ M, no more than 3×10⁻¹⁰ M, no more than 2×10⁻¹⁰ M, no more than 1×10⁻¹⁰ M, no more than 0.5×10⁻¹⁰ M) as measured by ATPase activity assay;
i) specifically binding to human CD39 at a K_{D} value of no more than 4×10⁻¹⁰ M (e.g. no more than 3×10⁻¹⁰ M, no more than 2×10⁻¹⁰ M, no more than 1×10⁻¹⁰ M, or no more than 0.5×10⁻¹⁰ M) as measured by Octet assay;
j) specifically binding to human TGFβ1 at a K_{D} value of no more than 4×10⁻¹¹ M (e.g. no more than 3×10⁻¹¹ M, no more than 2×10⁻¹¹ M, no more than 1×10⁻¹¹ M, or no more than 0.5×10⁻¹¹ M) as measured by Octet assay;
k) capable of recovering T cell function as measured by a Treg suppression assay;
l) capable of inhibiting human T cell apoptosis in a dose-dependent way;
m) capable of promoting human T cell survival and activation over stimulation;
n) capable of blocking TGFβ induced Foxp3 expression on total T cells; and
o) capable of restoring ATP induced inhibition on human T cell proliferation.

65. The conjugate molecule of any one of the preceding claims, which further comprises one or more conjugate moieties.

66. The conjugate molecule of claim 65, wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety or other anticancer drugs.

67. A pharmaceutical composition comprising the conjugate molecule of any one of the preceding claims, and one or more pharmaceutically acceptable carriers.

68. An isolated polynucleotide encoding the conjugate molecule of any one of claims 1-67.

69. A vector comprising the isolated polynucleotide of claim 68.

70. A host cell comprising the vector of claim 69.

71. A kit comprising the conjugate molecule of any one of claims 1-66 and/or the pharmaceutical composition of claim 67, and a second therapeutic agent.

72. A method of expressing the conjugate molecule of any one of claims 1-66, comprising culturing the host cell of claim 70 under the condition at which the vector of claim 69 is expressed.

73. A method of treating, preventing or alleviating a CD39 related and/or a TGFβ related disease, disorder or condition in a subject, comprising administering to the subject a therapeutically effective amount of the conjugate molecule of any one of claims 1-66 and/or the pharmaceutical composition of claim 67.

74. A method of treating, preventing or alleviating a disease treatable by reducing the ATPase activity of CD39 in a subject, comprising administering to the subject a therapeutically effective amount of the conjugate molecule of any one of claims 1-66 and/or the pharmaceutical composition of claim 67.

75. A method of treating, preventing or alleviating a disease associated with adenosine-mediated inhibition of T, Monocyte, Macrophage, DC, APC, NK and/or B cell activity in a subject, comprising administering to the subject a therapeutically effective amount of the conjugate molecule of any one of claims 1-66 and/or the pharmaceutical composition of claim 67.

76. A method of treating, preventing or alleviating a disease associated with an increased level and/or activity of TGFβ in a subject, comprising administering to the subject a therapeutically effective amount of the conjugate molecule of any one of claims 1-66 and/or the pharmaceutical composition of claim 67.

77. The method of any one of claims 73-76, wherein the disease, disorder or condition is cancer, pancreatic atrophy, or fibrosis.

78. The method of claim 77, wherein the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, or adenocarcinoma.

79. The method of claim 77, wherein the cancer is leukemia, lymphoma, bladder cancer, glioma, glioblastoma, ovarian cancer, melanoma, prostate cancer, thyroid cancer, esophageal cancer or breast cancer.

80. The method of any one of claims 73-76, wherein the subject has been identified as having a cancer cell or tumor infiltrating immune cells or immune suppression cells expressing CD39 and/or TGFβ, optionally at a level significantly higher from the level normally found on non-cancer cells or non-immune suppression cells.

81. The method of claim 80, wherein the immune suppression cells are regulatory T cells.

82. The method of claim 81, wherein the subject has been identified as having an overactive regulatory T cell in tumor microenvironment compared to the activity of a regulatory T cell normally found in a control subject.

83. The method of claim 81 or 82, wherein the subject is expected to be beneficial from the reversion of immunosuppression, or the reversion of dysfunctional exhausted T cells.

84. The method of any one of claims 73-76, wherein the disease, disorder or condition is an autoimmune disease or infection.

85. The method of claim 84, wherein the autoimmune disease is immune thrombocytopenia, systemic scleroderma, sclerosis, adult respiratory distress syndrome, eczema, asthma, Sjogren's syndrome, Addison's disease, giant cell arteritis, immune complex nephritis, immune thrombocytopenic purpura, autoimmune thrombocytopenia, Celiac disease, psoriasis, dermatitis, colitis or systemic lupus erythematosus.

86. The method of claim 84, wherein the infection is HIV infection, HBV infection, HCV infection, inflammatory bowel disease, or Crohn's disease.

87. The method of any one of claims 73-86, wherein the subject is human.

88. The method of any one of claims 73-87, wherein the administration is via oral, nasal, intravenous, subcutaneous, sublingual, or intramuscular administration.

89. The method of any one of claims 73-88, further comprising administering a therapeutically effective amount of a second therapeutic agent.

90. The method of claim 89, wherein the second therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

91. A method of modulating CD39 activity in a CD39-positive cell, comprising exposing the CD39-positive cell to the conjugate molecule of any one of claims 1-66 and/or the pharmaceutical composition of claim 67.

92. The method of claim 91, wherein the CD39-positive cell is an immune cell.

93. Use of the conjugate molecule of any one of claims 1-66 and/or the pharmaceutical composition of claim 67 in the manufacture of a medicament for treating, preventing or alleviating a CD39 related or a TGFβ related disease, disorder or condition in a subject.
